(19) United States
(12) Patent Application Publication
(10) Pub. No.: US 2010/0162416 A1
(43) Pub. Date: Jun. 24, 2010

(54) Multi-stage stem cell carcinogenesis

(75) Inventors: Ana Krtolica, San Francisco, CA (US); Dusko Ilic, San Francisco, CA (US)

Correspondence Address: WILSON, SONSINI, GOODRICH & ROSATI, 650 PAGE MILL ROAD, PALO ALTO, CA, 94304-1050, US

(73) Assignee: StemLifeLine, Inc., San Carlos, CA (US)

(21) Appl. No.: 12/587,005

(22) Filed: Sep. 29, 2009

Related U.S. Application Data
(60) Provisional application No. 61/101,069, filed on Sep. 29, 2008.

(51) Int. Cl. A61K 49/00; C12N 15/01; C12N 5/071; C40B 40/00; C40B 50/02; C40B 30/02; A01K 67/027; A61P 35/00

(52) U.S. Cl. 800/3; 435/441; 435/366; 506/13; 506/24; 506/8; 424/9.1; 800/21

(57) ABSTRACT

The present invention relates to a system of multi-stage stem cell carcinogenesis and a method of generating such multi-stage stem cell carcinogenesis system. Various stages of cancer stem cells can be generated from normal stem cells via mutagenesis. The system of the present invention enables monitoring changes in the ability of cells to transition from one stage of carcinogenesis to another and to identify genetic pathways and molecules that influence carcinogenesis. The present invention also enables a high-throughput and nonbiased screening for targets that preferentially affect cancer stem cells relative to non-cancer stem cells or their derivatives during stem cell carcinogenesis, thus is useful in developing anti-cancer therapeutics.

### CROSS REFERENCE

This application claims the benefit of U.S. Provisional Application No. 61/101,069, entitled “Multi-stage Stem Cell Carcinogenesis” filed Sep. 29, 2008 (Attorney Docket No. 37310-701.101), which is incorporated herein in its entirety by reference.

### BACKGROUND OF THE INVENTION

Cancer, also known as malignant neoplasm, is characterized by an abnormal growth of cells that display uncontrolled cell division, invasion and destruction of adjacent tissues, and sometimes metastasis to other locations in the body. There are more than 100 types of cancer, including breast cancer, skin cancer, lung cancer, colon cancer, prostate cancer, and lymphoma. Cancer is the second leading cause of death in America and it causes about 13% of all deaths. Cancer may affect people at all ages, even fetuses, but the risk for most types of cancer increases with age. Cancers can affect all animals.

Stem cells are cells capable of renewing themselves through mitotic cell division and differentiating into a diverse range of specialized cell types. There are two broad types of mammalian stem cells: embryonic stem cells that are found in blastocysts, and adult stem cells that are found in adult tissues. Cancer stem cells (CSCs) are a sub-population of cancer cells that possess characteristics normally associated with stem cells, such as self-renewal via symmetrical cell division and the ability to differentiate into multiple cell types and give rise to multiple cancer cell types, as well as indefinite life span due to telomerase activity and abbreviated cell cycle regulation. In addition, cancer stem cells are tumorigenic, i.e. cells capable of forming tumors from very small number of cells (at limiting dilutions), contributing to tumor growth. Thus, study of cancer stem cells is important for developing more effective preventive and therapeutic treatments of cancer.

### SUMMARY OF THE INVENTION

The present invention relates to compositions and methods for generating a multi-stage stem cell carcinogenesis system, and compositions and methods for treating, characterizing, and diagnosing cancer.

In one aspect, the present invention provides a method of generating cancer stem cells (CSC) or tumor initiating cells (TIC) comprising a) contacting a non-cancer stem cell or its derivatives with at least one mutagen; b) selecting stem cell clone that displays a transformed phenotype; c) optionally contacting the selected stem cell clone having a transformed phenotype with at least one mutagen; and d) selecting stem cell clones that display transformed phenotypes characteristic of different stages of stem cell malignant transformation. Transformed cells may or may not be exposed to mutagen for the second time. In some embodiments, the transformed cells are isolated after the first exposure of mutagenesis. In some embodiments, the non-cancer stem cell is a human stem cell. In some embodiments, the non-cancer stem cell is a human embryonic stem cell, a human adult stem cell, a human neural stem cell, or a derivative thereof. In some embodiments, the non-cancer stem cells or their derivatives have the same genetic origin as the CSC/TIC. The cancer stem cell of the present invention can be an early premalignant cell, a premalignant cell, or a malignant cell. Examples of the mutagens that can be used in the present invention include but are not limited to ICR191, 9-aminoacridine, ICR364-OH, ICR170, nitrosoguanidine, diethylsulfate, any member of an acridine family of mutagens or a derivative thereof. In some embodiments, the transformed phenotype comprises cell morphology, cell growth, cell differentiation, cell polarization, cell adhesion, cell migration, or cell invasiveness. The stage of stem cell malignant transformation can be an unaltered state, early premalignant state, premalignant state, malignant state, or an advanced malignant state of a stem cell.

In another aspect, the present invention provides a method of generating isogenic human CSC or TIC comprising deriving human CSC from human non-cancer stem cells of the same genetic origin. In some embodiments, the non-cancer stem cell is a human embryonic stem cell, a human adult stem cell, a human neural stem cell, a human progenitor cell, or a differentiated derivative thereof. In some embodiments, the cancer stem cell is an early premalignant cell, a premalignant cell, or a malignant cell. The genetic origin can be a type of variation selected from the group consisting of ethnicity, genetic polymorphism, predisposition to a disease, and genetic mutation. In some embodiments, the deriving of the CSC or TIC comprises the steps of contacting a non-cancer stem cell with at least one mutagen; selecting stem cell clones that display a transformed phenotype; optionally contacting the selected stem cell clones having a transformed phenotype with at least one mutagen; selecting stem cell clones that display transformed phenotypes characteristic of different stages of stem cell malignant transformation; and isolating cells from tumor that is generated via in vivo tumorigenesis. Transformed cells do not necessarily need to be again exposed to mutagen. In some embodiments, the transformed cells are isolated after the first exposure of mutagenesis. The mutagens that can be used in the subject methods can be ICR191, 9-aminoacridine, ICR364-OH, ICR170, nitrosoguanidine, diethylsulfate, or any member of an acridine family of mutagens or a derivative thereof. In some embodiments, the transformed phenotype comprises cell morphology, cell growth, cell differentiation, cell polarization, cell adhesion, cell migration, or tissue invasiveness. In some embodiments, the stage of stem cell malignant transformation comprises an unaltered state, early premalignant state, premalignant state, malignant state, or an advanced malignant state of a stem cell.

In another aspect, the present invention provides a system for modeling and diagnosis of stem cell malignant transformation (carcinogenesis) comprising: a) non-cancer stem cell or its derivatives; b) isogenic non-cancer stem cell-derived cancer stem cells or tumor initiating cells with transformed phenotypes characteristic of each stage of stem cell malignant transformation; and c) a database with information on stem cell malignant transformation. In some embodiments, the non-cancer stem cell is a human stem cell. In some embodiments, the non-cancer stem cell is a human embryonic stem cell, a human adult stem cell, a human neural stem cell, or a derivative thereof. The cancer stem cell can be an early premalignant cell, a premalignant cell, or a malignant cell. In some embodiments, the cancer stem cell and the non-cancer stem cell have the same genetic origin. In some embodiments, the transformed phenotype comprises cell morphology, cell growth, cell differentiation, cell polarization, cell adhesion, cell migration, or tissue invasiveness. The stage of stem cell malignant transformation may comprise an unaltered state, early premalignant state, premalignant state, malignant state, or an advanced malignant state of a stem cell. The database of the MSCC model of the present invention may contain information on molecular and/or cellular events or characteristics associated with stem cell malignant transformation. In some embodiments, the molecular and/or cellular event can be a change in expression of a gene, enzymatic activity, telomerase activity, genomic stability, chromosomal modification, mutation, epigenetic change, cell growth, cell differentiation, cell polarization, cell adhesion, cell migration, or tissue invasiveness.

In another aspect, the present invention provides a method of profiling stem cell malignant transformation comprising a) generating stage-specific cancer stem cell from an isogenic non-cancer stem cell or its derivatives; b) identifying molecular and/or cellular characteristics associated with each stage of stem cell malignant transformation; and c) generating a database with information on molecular and cellular characteristics of stem cell malignant transformation. In some embodiments, the non-cancer stem cell is a human stem cell. In some embodiments, the non-cancer stem cell is a human embryonic stem cell, a human adult stem cell, a human neural stem cell, or a derivative thereof. In some embodiments, the cancer stem cell is an early premalignant cell, a premalignant cell, or a malignant cell. The cancer stem cell and the non-cancer stem cell may have the same genetic origin. The transformed phenotype may include but is not limited to cell morphology, cell growth, cell differentiation, cell polarization, cell adhesion, cell migration, or tissue invasiveness. The stage of stem cell malignant transformation can be an unaltered state, early premalignant state, premalignant state, malignant state, or an advanced malignant state of a stem cell. In some embodiments, the molecular and cellular event associated with stem cell malignant transformation includes but is not limited to a change in expression of a gene, enzymatic activity, telomerase activity, genomic stability, chromosomal modification, mutation, epigenetic change, cell growth, cell differentiation, cell polarization, cell adhesion, cell migration, or tissue invasiveness. In some embodiments, the subject method further comprises identifying molecular or cellular changes through short hairpin RNA (shRNA) gene expression.

In another aspect, the present invention provides a method of detecting a cancerous or pre-cancerous cell comprising: a) obtaining a cell (e.g. tumor/cancer cell/tissue, normal cell or stem cell) from a subject; b) assessing molecular and cellular profile of the stem cell from the subject; and c) comparing the profile of the stem cell from the subject with a database of multi-stage stem cell carcinogenesis of claim 26. In some embodiments, the subject is a human. In some embodiments, the subject is a human pre-diagnosed or diagnosed with cancer. In some embodiments, the stem cell is a human adult stem cell. In some embodiments, the stem cell is a normal cell, an early premalignant cell, a premalignant cell, a malignant cell, or an advanced malignant cell. The profile of the stem cells may comprise properties of a normal untransformed stem cell. Alternatively, the profile of the stem cells can comprise one or more tumorigenic properties including but not limited to cell morphology, cell growth, cell differentiation, cell polarization, cell adhesion, cell migration, or tissue invasiveness. The database of multi-stage stem cell carcinogenesis may contain information on molecular and/or cellular characteristics associated with each stage of stem cell malignant transformation. In some embodiments, the stage of stem cell malignant transformation comprises an unaltered state, early premalignant state, premalignant state, malignant state, or an advanced malignant state of a stem cell.

In another aspect, the present invention provides a method for screening agent that preferentially affects a cancer stem cell relative to an isogenic non-cancer stem cell or its derivatives comprising: a) administering a biologically active agent to a cancer stem cell and an isogenic non-cancer stem cell or its derivatives; and b) selecting an agent that exhibits a differential biological effect on the cancer stem cell than on the isogenic non-cancer stem cell or its derivatives. The agent of the subject method can be a small molecule, an antibody-based agent, or an RNA. The cancer stem cell can be at any stage of stem cell carcinogenesis, i.e. early premalignant state, premalignant state, malignant state, or an advanced malignant state. In some embodiments, the agents are screened using three dimensional cell culture system. In some embodiments, the biological effect is a change in a biological response comprising cell proliferation, apoptosis, cell differentiation, cell migration, cell motility, cell polarization, cell adhesion, cell cytotoxicity, and/or cell cytokine secretion. The screening of the agent can be performed in vitro or in vivo.

In yet another aspect, the present invention provides a method of generating neural stem cells (NSC) from human embryonic stem cells (hESC), comprising: a) culturing hESC; b) disrupting cell cell contact by making single-cell suspension; c) seeding single cells onto human xeno-free defined extracellular matrix (ECM) substrate; d) disaggregating the hESC colonies and disrupting cell-cell contact; e) growing disaggregated cells in a NSC culture medium in the presence of basic fibroblast growth factor (bFGF); and f) dissociating adherent cells and replating the dissociated cells onto the NSC culture medium in the presence of bFGF for several passages to obtain NSC. In some embodiments, the cells are disaggregated with accutase. In some embodiments, the NSC culture medium is StemPro NSC SFM medium. In some embodiments, the generated NSC express early neural lineage markers and pluropotency markers including but not limited to nestin, Oct 3/4, Sox1, βIII tubulin, and radial glial protein 3CB₂. In some embodiments, the ECM substrate is feeder cell-free. The subject method may further comprise seeding the obtained NSC onto poly-L-ornithine/laminin-coated surface in StemPro NSC SFM without bFGF for six weeks to induce differentiation of NSC into neural cell types. The obtained NSC are capable of differentiating into neurons, astrocytes, and oligodendrocytes. In some embodiments, the obtained NSC are mutagenized to give rise to cancer NSC.

In still another aspect, the present invention provides a method of generating an animal model of tumorigenesis comprising administering to an animal CSC/TIC cells expressing stable CSC/TIC phenotype generated via the subject method of the present invention. The animal model of the present invention can be used for screening compounds that modulate tumor growth in vivo. In some embodiments, the animal is an immunocompromised host. The tumor arising from the CSC/TIC cells typically bears substantially the same molecular or cellular characteristics as the CSC/TIC cells administered to the animal. In some embodiments, the CSC/TIC cells are differentiated. In one example, the CSC/TIC cells are transformed neural stem cells (NSC). In some embodiments, the CSC/TIC cells are administered to a specific tissue or site of the animal. The CSC/TIC cells administered at a specific tissue or site can give rise to tissue-specific tumor in the animal. In some embodiments, the CSC/TIC cells are introduced with a reporter gene. In some embodiments, the subject method further comprises administering hESC-derived stromal cells in combination with CSC/TIC cells.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the differentiation pathway of a human embryonic stem cell (hECS) capable of self-renewal. Cancer stem cells (CSC) or tumor initiating cells (TIC) may develop when self renewing normal (i.e. non-cancerous) stem cells acquire mutations and are genetically altered or transformed. TIC/CSC share with embryonic stem cells (ESC) multiple characteristics. Upon mutagenesis, CSC or TIC are not only able to self-renew, but may also lose ability to respond to growth inhibitory and differentiation signals and acquire malignant phenotypes.

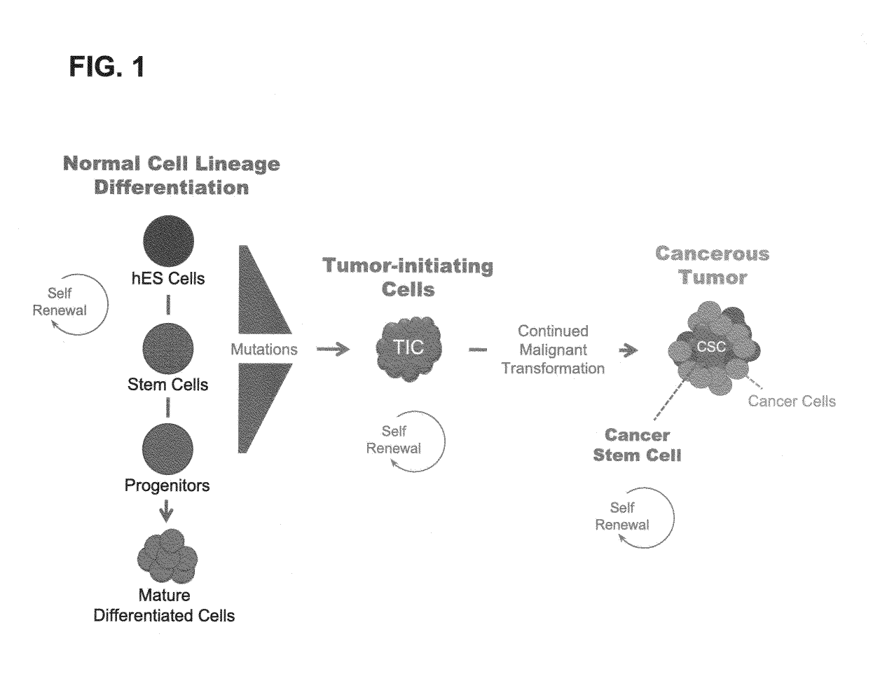

FIG. 2 is a schematic diagram of the multi-stage/level targeted intervention of stem cell carcinogenesis. Cancer stem cells are generated at various stages of stem cell differentiation, i.e. from hESC to lineage specific stem cells to progenitors. Functional genomic, proteomic and chemical screens at various stages of stem cell carcinogenesis enable the identification of unique and abnormal pathways specifically involved in cancer stem cell development. The platform of the present invention allows inducing cell death, inhibition or reversal of tumorigenicity, and initiation or activation of differentiation.

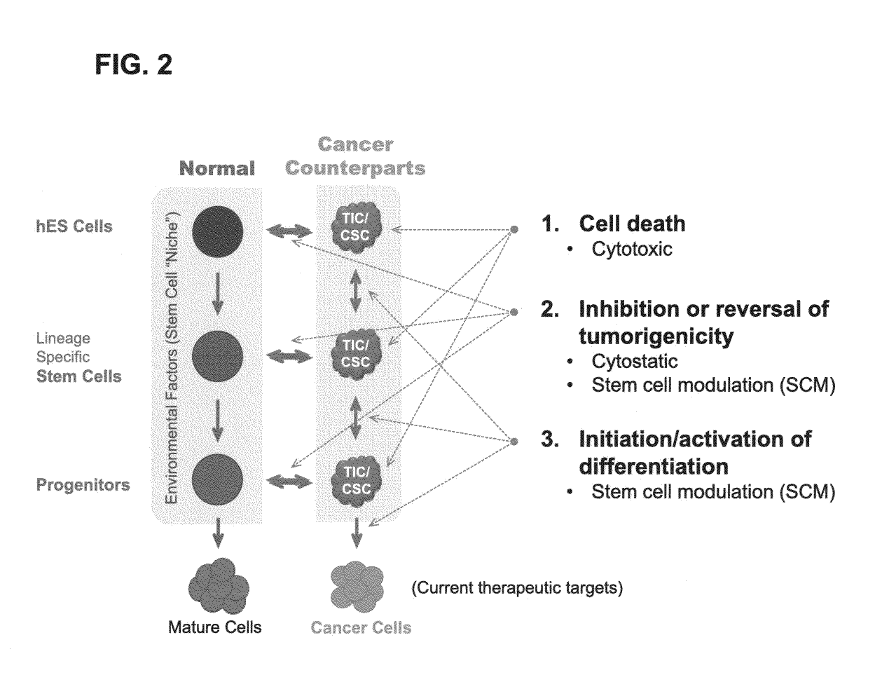

FIG. 3 is a schematic diagram showing multi-stage stem cell carcinogenesis (MSCC) platform: a novel genomic pathway-based, controlled and integrated cancer stem cell drug discovery/screening platform that mirrors carcinogenesis and the cancer disease process. In this model, normal cells and TIC/CSC are cloned at various or multiple stages of carcinogenesis. Selected drugs are screened against comprehensively profiled TIC/CSC. Animal/tumor models are developed for specific cell/tissue or cancer types.

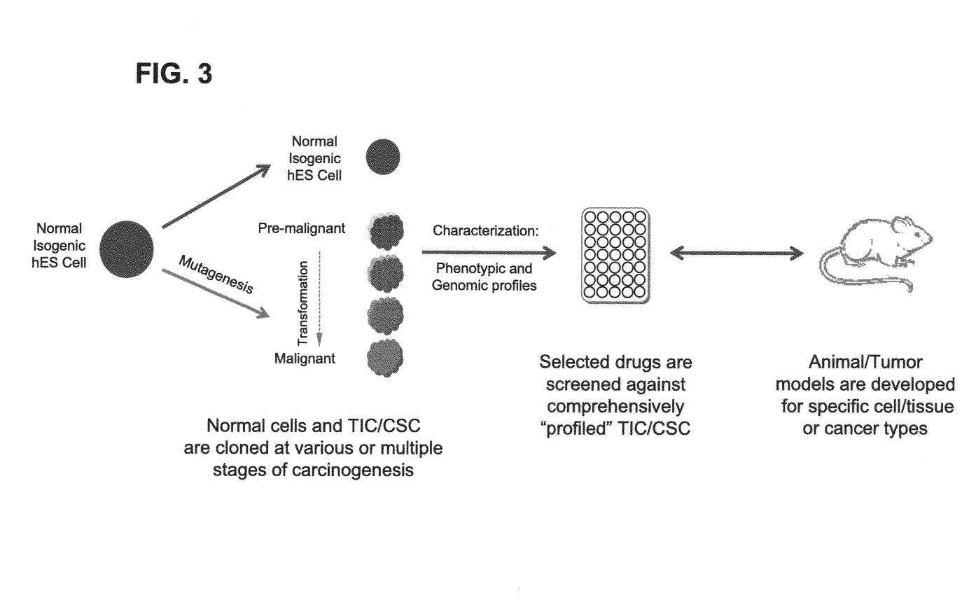

FIG. 4 shows in vivo MSCC model. There are two exemplary non-limiting approaches for the in vivo tumor model. The first approach is a universal model in which PAN-acting anticancer drugs that target universal/core signature of the genomically profiled TIC/CSC. The second approach is a tissue specific anticancer drugs that target genomically profiled primary or metastatic TIC/CSC in the context of microenvironment or niche-induced phenotypes.

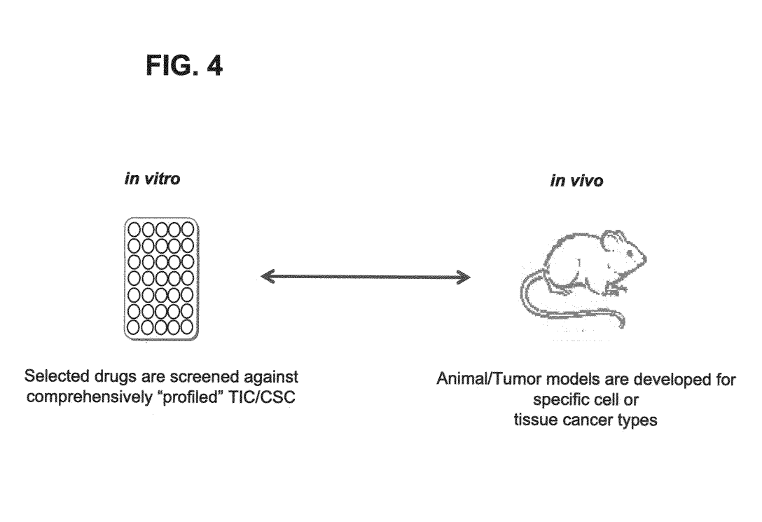

FIG. 5 shows that the MSCC technology of the present invention can be used to characterize mutagenesis. Normal stem cells and TIC/CSC are cloned and characterized for genomic profiling. Drugs are then screened against the profiled TIC/CSC.

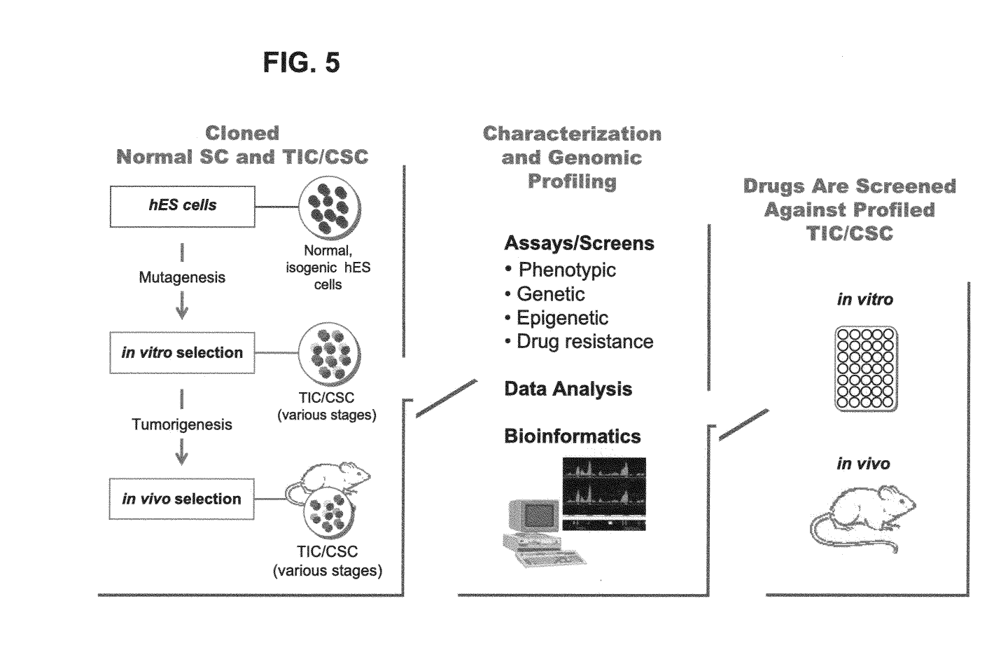

FIG. 6 shows that normal hESC can differentiate along several pathways to give rise to neural stem cells (NSC) which then give rise to differentiated cells including neurons, glia, and astrocytes. hESC can also go through malignant transformation to give rise to additional mutant lines or go through partial differentiation to give rise to, for example, NSC.

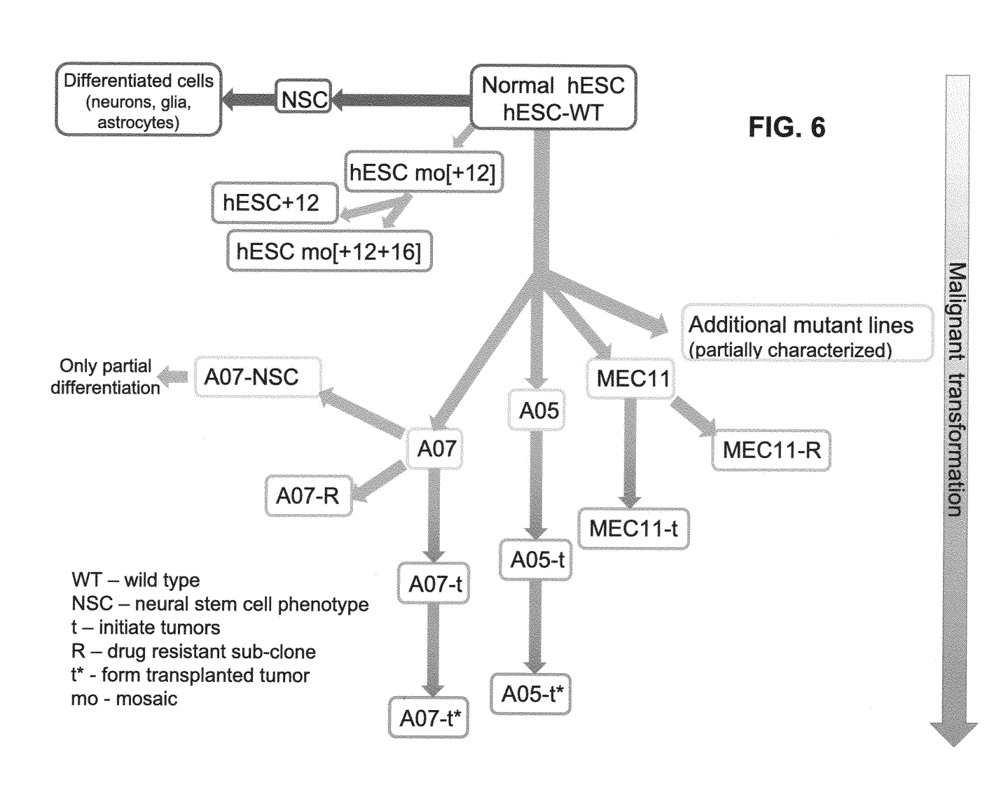

FIG. 7 shows that mutated stem cells do not respond to differentiation signals. FIGS. 7A and B show that the ICR191 induced mutated hESC maintain hESC morphology after 4 passages in the presence of the differentiation media (differentiation medium 1 as shown in FIG. 7A contains 10% fetal calf serum FCS; differentiation medium 2 as shown in FIG. 7B contains 10% FCS without human fibroblast growth factor (hFGF)). FIG. 7C shows that the wildtype hESC differentiate within the first passage after exposure to the differentiation medium 1 containing 10% FCS. FIG. 7D shows that the wildtype hESC grown in the standard medium (i.e. without differentiation factors) do not differentiate spontaneously and can not be morphologically distinguished from the ICR191 mutated hESC that have been grown in the presence of the differentiation media 1 and 2 as shown in FIGS. 7A and B. Micrographs are taken at 100× magnification.

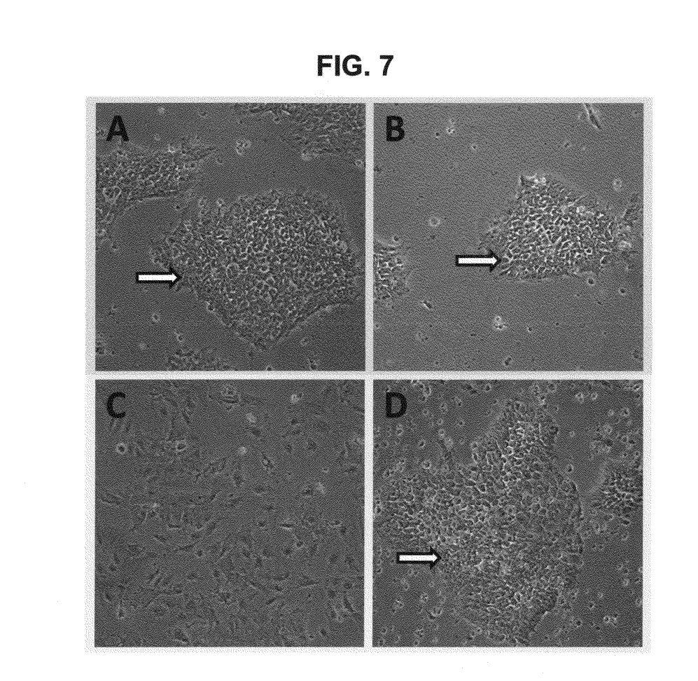

FIG. 8 shows growth of the isolated MSCC clones in methyl cellulose assay (blue). All clones show higher ability for anchorage-independent growth than normal hESC (black). Two cancer cell lines served as positive controls and are shown for comparison (orange). B—Examples of growing clone (left) and arrested hESC (right).

FIG. 9 shows examples of a clone with highly invasive (left), moderately invasive (middle), and little invasive (right) phenotype in Matrigel-assay.

FIG. 10 shows the proliferation rate of the selected clones. FIG. 16A shows BrdU incorporation (fluorescent units per 1000 cells) for parental hESC and selected clones. FIG. 16B shows doubling times for the same cell lines as calculated during exponential culturing.

FIG. 11 shows percentage of parental hESC cells and three clones (05, 07 and 11) that formed colonies in methyl-cellulose after 3 days in culture.

FIG. 12 shows proliferation rate of hESC and clones: 05, 07 and 11 in the complete, bFGF-supplemented and growth factor (GF)-free medium as measured by BrdU incorporation.

FIG. 13 shows immunolocalization of ABCB5, ABCG2, CD90, ESA and CD133 cancer stem cell markers in clones 05, 07 and 11.

FIG. 14 shows IC50 values for selected clones 05 (red), 07 (green) and 11 (violet) expressed as a fold increase relative to the isogenic hESC control.

FIG. 15 shows dose response curves for temozolomide (MTIC), chlorambucil (CLB), irinotecan (SN-38) and carmustine (BCNU) for clones 05 (red), 07 (green) and 11 (violet) as compared to the isogenic hESC control.

FIG. 16 shows dose response curves for salinomycin as measured by BrdU incorporation. Clones 05 (red) and 07 (green) are significantly more affected than clone 11 (violet), isogenic hESC control (blue), or mature isogenic cancer lines derived from tumors, TMEC02 (light blue) and TMEC03 (bright green).

FIG. 17 shows tumor formation in immunocompromised mice. A—1 million and 100,000 cells of clones 07 (light and dark olive green, respectively), 11 (light and dark violet, respectively) and 05 (light and dark red, respectively) were injected subcutaneously into immunocompromised mice. B, C—Freshly isolated tumors were photographed and weighted.

FIG. 18 shows formation of transplanted tumors in immunocompromised mice. 100,000 and 10,000 cells isolated from the primary tumor formed by clone 07 were re-injected subcutaneously into immunocompromised mice.

FIG. 19 shows H&E staining of tumor sections depicting tumor cells invading surrounding host adipose tissue (black arrow shows direction of the invasion; white arrows point to invading cells) confirming malignant phenotype.

FIG. 20 shows differentiation of hESC into NSC. Unlike parental ESC line, generated NSC express radial glial protein (RGP; red) and Sox1 (green), markers of NSC. Hoechst (blue) is a nuclear stain. To ensure that there is no antigen-specific fluorescence, red (top) and green (bottom) images on the left were overexposed.

FIG. 21 shows multipotent nature of NSC. Differentiation into the three main cell types of central nervous system has been evaluated using immunocytochemistry. Myelin basic protein (MBP; red)-positive oligodendrocytes were detected in the 6-week old culture along with glial fibrillar acidic protein (GFAP; red)-positive astrocytes and neuronal nuclear protein (NeuN; green), peripherin (not shown) and DM tublin-positive (red) neurons. Hoechst (blue) is a nuclear stain.

FIG. 22 shows differentiation of mutant clones into NSC. While A07 cells converted to neural-like stem cells express markers of NSC, radial glial protein (red) and Sox1 (green), parental clone ICR-A07 does not (left panel). Hoechst (blue) is a nuclear stain.

### DETAILED DESCRIPTION OF THE INVENTION

The present invention provides compositions and methods for generating a multi-stage stem cell carcinogenesis system. In general, the compositions and methods for generating a multi-stage stem cell carcinogenesis system are directed toward generating cancer stem cell lines from normal stem cells, profiling the cancer stem cell lines generated at various stages of stem cell carcinogenesis, and developing anti-cancer therapeutics based on information derived from the multi-stage stem cell carcinogenesis system.

The methods of the invention include the generation of cancer stem cells from normal unaltered stem cells via mutagenesis, characterization of cancer stem cell clones generated at various stages of malignant transformation for their tumorigenic and malignant phenotypes including gene expression profiles. The methods of the invention further include the generation of large quantity of homogeneous cancer stem cells from non-cancer stem cells of the same genetic origin. Also, described herein are methods of identifying genetic pathways and molecules that are involved in the transition from one stage of the stem cell carcinogenesis to another along the stem cell carcinogenesis continuum. Further, the present invention includes compositions and methods of screening for agents that preferentially affect the cancer stem cells relative to the normal stem cells and the methods of using such agents to treat cancer by preferentially modulating cancer stem cells over normal stem cells. Such modulation of cancer stem cells may include but is not limited to inhibition of cancer stem cells.

The present invention also provides compositions and methods of generating a system of stem cell carcinogenesis. Such system includes but is not limited to cancer stem cells generated at various stages of stem cell carcinogenesis, molecules associated with various stages of stem cell carcinogenesis, and agents that preferentially target cancer stem cells relative to normal untransformed stem cells or their derivatives. One embodiment of the invention is a systems biology database for multi-stage stem cell carcinogenesis including but not limited to information on cellular properties of cancer stem cells at various stages of carcinogenesis, pathways and molecules involved in various stages of stem cell carcinogenesis, molecules altered in premalignant and malignant stages relative to normal unaltered stem cells, and agents that preferentially affect cancer stem cells at various stages of carcinogenesis relative to normal unaltered stem cells.

In one aspect the present invention includes methods of generating cancer stem cells from unaltered non-cancer stem cells, such as embryonic stem cells and neural stem cells and the derivatives thereof. In one embodiment the methods of generating cancer stem cells from unaltered non-cancer stem cells involve mutagenesis. Exposure to one or more rounds of mutagenesis results in the generation of cancer stem cell lines that represent different stages of stem cell carcinogenesis.

### I. Cancer

Cancer, known as malignant neoplasm, is a class of diseases in which abnormal cells divide without control and are able to invade other tissues and spread to other parts of the body, known as metastasis. Cancer cells can spread to other parts of the body through the blood and lymph systems. These three malignant properties of cancers, i.e. uncontrolled cell growth, invasion and metastasis, differentiate them from benign tumors, which are self-limited, do not invade or metastasize. Most cancers form a tumor, i.e. solid tumor, but some types, such as leukemia, do not.

There are more than 100 different types of cancer. Most cancers are named for the organ or type of cell in which they originate—for example, cancer that originates in the colon is called colon cancer; cancer that originates in basal cell of the skin is called basal cell carcinoma. Cancer types can be grouped into broader categories. The main categories of cancer include but are not limited to: Carcinoma: cancer that originates in the skin or in tissues that line or cover internal organs; sarcoma: cancer that originates in bone, cartilage, fat, muscle, blood vessels, or other connective or supportive tissue; leukemia: cancer that originates in blood-forming tissue such as the bone marrow and causes large numbers of abnormal blood cells to be produced and enter the blood; lymphoma and myeloma—cancers that originate in the cells of the immune system; central nervous system cancers: cancers that originate in the tissues of the brain and spinal cord.

Transformation is referred to as the change that a normal cell undergoes as it becomes malignant. Nearly all cancers are caused by abnormalities in the genetic material of the transformed cells. These abnormalities may be due to the effects of carcinogens, such as tobacco, radiation, chemicals, or infectious agents. Certain types of cancer are inherited, and thus present in all cells from birth. The heritability of cancers is usually affected by complex interactions between carcinogens and the host's genome. Other cancer-promoting genetic abnormalities may be randomly acquired through errors in DNA replication, DNA methylation, and microRNA.

Genetic abnormalities found in cancer typically affect two general classes of genes. The first class includes oncogenes, i.e. cancer-promoting genes, which when deregulated, participate in the onset and development of cancer. Genetic mutations resulting in the activation of oncogenes gives those cells new properties, such as hyperactive growth and division, protection against apoptosis, which is programmed cell death, loss of respect for normal tissue boundaries, and the ability to become established in diverse tissue environments. Changes in these properties are part of the cell carcinogenesis process. The second class of genes affecting the onset of cancer includes tumor suppressor genes, which are inactivated in cancer cells, resulting in the loss of normal functions in those cells, such as accurate DNA replication, control over the cell cycle, orientation and adhesion within tissues, and interaction with protective cells of the immune system.

Diagnosis and treatment of cancer is related to its stage. The stage of a cancer refers to how much it has grown and whether the tumor has spread from its original location. Specifically, the stage often takes into account the size of a tumor, how deep it has penetrated, whether it has invaded adjacent organs, how many lymph nodes it has metastasized to if any, and whether it has spread to distant organs. Once diagnosed, cancer is usually treated with a combination of surgery, chemotherapy and radiotherapy. Advances in cancer research have allowed more specific treatments for different varieties of cancer. There has been significant progress in the development of targeted therapy drugs that act specifically on detectable molecular abnormalities in certain tumors, and which minimize damage to normal cells. The prognosis of cancer patients is most influenced by the type of cancer, as well as the stage of the disease. In addition, the presence of specific molecular markers can also be useful in establishing prognosis, as well as in determining individual treatments.

### II. Stem Cells: Embryonic Stem Cells and Adult Stem Cells

Stem cells are characterized by the ability to renew themselves through symmetrical cell division, which means that the cell division generates two equal-size daughter cells that possess the same components and will develop into same cell type. Stem cells are capable of differentiating into a diverse range of specialized cell types, such as muscle cells, blood cells, or nerve cells. This property is known as pluripotency. Stem cells have the ability to regenerate tissue over a lifetime. The standard test for a bone marrow stem cell is the ability to transplant one cell without HSCs. In this case, a stem cell must be able to produce new blood cells and immune cells over a long term, demonstrating pluripotency. The two broad types of mammalian stem cells are: embryonic stem cells that are located in the inner mass of a blastocyst, and adult stem cells that are found in adult tissues. The embryos from which human embryonic stem cells are derived are typically four or five days old and are a hollow microscopic ball of cells called the blastocyst. The blastocyst includes three structures: the trophoblast, which is the layer of cells that surrounds the blastocyst; the blastocoel, which is the hollow cavity inside the blastocyst; and the inner cell mass, which is a group of approximately 30 cells at one end of the blastocoel. In a developing embryo, stem cells can differentiate into all of the specialized embryonic tissues and eventually give rise to every cell type of the adult organism. In adult organisms, stem cells and progenitor cells act as a repair system for the body, replenishing specialized cells, but also maintain the normal turnover of regenerative organs, such as blood, skin or intestinal tissues.

In one embodiment the stem cells used are embryonic stem cells. Embryonic stem (ES) cells are pluripotent. They are able to differentiate and give rise to cells that originate from all three germ layers: ectoderm, endoderm and mesoderm. These include each of the more than 220 cell types in the adult body, when ES cells are given sufficient and necessary stimulation for a specific cell type. Pluripotency distinguishes ES cells from multipotent progenitor cells found in the adult. Multipotent progenitor cells only form a limited number of cell types. When given no stimuli for differentiation, i.e. when grown in vitro, ES cells maintain pluripotency through multiple cell divisions. ES cells are capable of unlimited expansion in vitro and are considered an immortal epiblast derivative with a checkpoint in differentiation that enables their expansion as undifferentiated colonies that can be instructed to maintain this phenotype indefinitely (Trousnon, Endocrine Reviews, 27(2): 208-219, 2006).

Because of their plasticity and potentially unlimited capacity for self-renewal, ES cell therapies have been proposed for regenerative medicine and tissue replacement after injury or disease. In particular, embryonic cell lines and autologous embryonic stem cells generated through therapeutic cloning are considered as promising candidates. Stem cells, directed to differentiate into specific cell types, offer the possibility of a renewable source of replacement cells and tissues to treat diseases including Parkinson's and Alzheimer's diseases, spinal cord injury, stroke, burns, heart disease, diabetes, osteoarthritis, and rheumatoid arthritis. In addition, human stem cells could also be used to test new drugs. For example, new medications could be tested for safety on differentiated cells generated from human pluripotent cell lines.

In another embodiment the stem cells are adult stem cells. Adult stem cells, also known as somatic stem cells, are undifferentiated cells found throughout the body after embryonic development that divide to replenish dying cells and regenerate damaged tissues, and can differentiate to yield the major specialized cell types of the tissue or organ. Adult stem cells can be found in children as well as adults. Adult stem cells have been identified in many organs and tissues. However, there are a very small number of stem cells in each tissue. Stem cells are thought to reside in a specific area of each tissue where they may remain quiescent, i.e. non-dividing for many years until they are activated by disease or tissue injury. The adult tissues reported to contain stem cells include brain, bone marrow, peripheral blood, blood vessels, skeletal muscle, skin and liver.

There are several major types of adult stem cells including but not limited to adipose derived adult stem cells, induced pluripotent stem cells derived from epithelial cells, hematopoietic stem cells, mammary stem cells, mesenchymal stem cells, endothelial stem cells, neural stem cells, olfactory adult stem cells, testicular cells, and dental pulp derived stem cells. Scientific efforts have been made to grow adult stem cells in culture and manipulate them to generate specific cell types so they can be used to treat injury or disease. Some examples of potential treatments include replacing the dopamine-producing cells in the brains of Parkinson's patients, developing insulin-producing cells for type I diabetes and repairing damaged heart muscle following a heart attack with cardiac muscle cells.

In a living animal, adult stem cells can divide for a long period and can give rise to mature cell types that have characteristic shapes and specialized structures and functions of a particular tissue. Examples of differentiation pathways of adult stem cells include but are not limited to the following. Hematopoietic stem cells give rise to all the types of blood cells: red blood cells, B lymphocytes, T lymphocytes, natural killer cells, neutrophils, basophils, eosinophils, monocytes, macrophages, and platelets. Bone marrow stromal cells give rise to a variety of cell types including bone cells i.e. osteocytes, cartilage cells i.e. chondrocytes, fat cells i.e. adipocytes, and other kinds of connective tissue cells such as those in tendons. Neural stem cells in the brain give rise to its three major cell types: nerve cells (neurons) and two categories of non-neuronal cells—astrocytes and oligodendrocytes. Epithelial stem cells in the lining of the digestive tract occur in deep crypts and give rise to several cell types: absorptive cells, goblet cells, Paneth cells, and enteroendocrine cells. Skin stem cells occur in the basal layer of the epidermis and at the base of hair follicles. The epidermal stem cells give rise to keratinocytes, which migrate to the surface of the skin and form a protective layer. The follicular stem cells can give rise to both the hair follicle and to the epidermis. Under special conditions tissue-specific adult stem cells can generate a spectrum of cell types of other tissues, even crossing germ layers (Filip S, English D and Mokry J 2004). This phenomenon is referred to as stem cell transdifferentiation or plasticity. Known examples of adult stem cell plasticity that include the following: hematopoietic stem cells may differentiate into three major types of brain cells (neurons, oligodendrocytes, and astrocytes), skeletal muscle cells, cardiac muscle cells, and liver cells. Bone marrow stromal cells may differentiate into cardiac muscle cells and skeletal muscle cells. Brain stem cells may differentiate into blood cells and skeletal muscle cells.

In another embodiment the stem cells used are neural stem cells (NSC). Neural stem cells exist not only in the developing mammalian nervous system but also in the adult nervous system of all mammalian organisms, including humans. Neural stem cells can also be derived from embryonic stem cells. Neural stem cells are commonly cultured in vitro as neurospheres, which are floating heterogeneous aggregates of cells containing a large proportion of stem cells. They can be propagated for extended periods of time and differentiated into both neuronal and glia cells. However, this behavior may be induced by the culture conditions in progenitor cells, which normally undergo a strictly limited number of replication cycles in vivo (Doetsch F, et. al, Neuron, 2002). Neural stem cells share many properties with hematopoietic stem cells. Remarkably, when injected into the blood, neurosphere-derived cells differentiate into various types of immune cells (Bjornson C R, et. al., Science, 1999). As compared to ES cells, neural crest stem cells from adults have several advantages: similar to ES cells, they have the innate ability to differentiate into many diverse cell types; they are easily accessible in the skin of adults; and the patient's own neural crest stem cells could be used for cell therapy. The latter avoids both rejection of the implant and graft-versus-host disease. Furthermore, studies in the mouse have shown that neural crest stem cells from adult hair follicles are able to differentiate into neurons, nerve supporting cells, cartilage and bone cells, smooth muscle cells, and pigment cells.

### III. In Vitro Culture and Differentiation of Stem Cells

Embodiments of the present invention include the differentiation and expansion of human embryonic stem cells (hESCs) and their neural stem cell derivatives (hNSC) in vitro. hESCs are isolated by transferring isolated blastomere, the inner cell mass or whole embryo into a plastic laboratory culture dish. Specifically, human ESCs can be formed from mechanically or immuno-surgically isolated inner cell mass of preimplantation-stage blastocysts or may also be derived from earlier morula-stage human embryos, or intact blastocysts, after the removal of the zona pellucida in an acidified solution or by enzymatic digestion in pronase. Very little difference exists in the efficiency of producing hESCs from these different stages of embryo development when grown on fibroblast feeder cells. Mouse embryonic stem cells (mESCs) may be maintained in the presence of leukemia inhibiting factor (LIF) in vitro without feeder cell support, but this is not the case for hESCs (Trounson A O, Reprod Fertil Dev 13:523-532, 2001). There is a wide range of feeder cells that are appropriate for the maintenance of hESCs, including murine fetal fibroblasts (e.g., STO cells) and human cell lines, including human embryonic fibroblasts, human uterine endometrium, human foreskin fibroblasts, human adult bone marrow cells, and differentiated hESCs. Other cell lines, including commercially available human cells, are also in use for the maintenance of hESC and may also be appropriate for deriving the hESC from human embryos. To date, the methods used successfully to establish hESC lines are well established in the art (Reubinoff B E, et. al. Nat Biotechnol 18:399-404, 2000). These methods involve the isolation of inner cell mass clusters from human blastocysts by immunosurgery and their coculture with mitotically inactivated murine embryonic fibroblasts (MEFs). The hESCs form typical colonies of undifferentiated cells that need to be passaged weekly or, more often, as mechanically dissected colonies of 10 cells or more. hESC lines have also been derived under feeder-free conditions using cell-free lysates of MEFs (Klimanskaya I, et. al. Lancet 365:1636-1641, 2005).

This invention embodies serum- and feeder-free conditions for culturing hESCs. Serum-free and feeder layer-free conditions have been reported for the long-term maintenance of hESCs (Amit M, et. al. Biol Reprod 70:837-845, 2004). hESCs has been grown in medium containing 15% serum replacement and growth factors, tumor growth factor (TGFβ1), leukemia inhibitory factor (LIF), basic fibroblast growth factor (bFGF), and fibronectin. After 47-50 passages, the hESCs maintained euploidy and pluripotentiality. A hESC line has also been established on plastic dishes coated with extracellular matrix, which was dried and sterilized, from mouse embryonic fibroblasts (Klimanskaya I, et. al. Lancet 365:1636-1641, 2005). The presence of bFGF is considered essential in these feeder-free culture systems. Human serum has also been used to coat tissue culture dishes, then dried and used for culture of hESCs in the presence of medium conditioned by hESC-derived mesenchymal cell types.

In one embodiment, the present invention provides generation of blastomere-derived hESC lines under virtually xeno-free conditions in complete absence of serum. Blastomeres can be extracted from a cleavage (8-cell) stage embryo in a similar manner as used in preimplantation genetic diagnostics. hESC can be derived from blastomeres under physiologic oxygen conditions without the need for serum supplementation. For example, the physiologic oxygen condition can be 5%, 5.5%, 6%, 6.5%, 7%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.5%, or 10% oxygen level. In one embodiment, the physiologic oxygen condition is 8.0%. The methods of producing hESC from blastomeres and/or their progeny are disclosed in U.S. patent application Ser. No. 12/455,954, which is herein incorporated by reference in its entirety.

The long-term stability of hESCs is an important issue. It is necessary to reassess karyotypes of hESCs regularly. Furthermore, there may be a need to monitor for mutations, especially the critical genes such as the oncogene family, which may influence differentiation and tumor formation in vivo. There are many tests that measure the fundamental properties of ES cells. These tests include but are not limited to growing and subculturing the ES cells using methods such as the clonogenic assays where single cells are characterized by their ability to differentiate and self-renew for a long period of time. Cell morphology can be monitored to ensure that the ES cells remain undifferentiated. Cell surface markers that are found only on undifferentiated cells are determined using specific techniques. Another important test of ES cells is for the presence of a protein called Oct-4, a transcription factor which undifferentiated cells typically make and an important regulator of cell differentiation and embryonic development. The pluripotent property of ES cells can be assessed by 1) allowing the cells to differentiate spontaneously in cell culture; 2) manipulating the cells so they will differentiate to form specific cell types; or 3) injecting the cells into an immunosuppressed mouse to test for the formation of a benign tumor called a teratoma. Teratomas typically contain a mixture of many differentiated or partly differentiated cell types, which indicates that the ES cells are capable of differentiating into multiple cell types.

As long as the embryonic stem cells in culture are grown under certain conditions, they can remain undifferentiated. They may be grown indefinitely in vitro while maintaining their original karyotype and epigenetic status, but the appropriate culturing conditions need to be provided. hESCs spontaneously differentiate in the absence of the appropriate cell feeder layer, provided appropriate growth factors and matrix, when overgrown in culture and when isolated from the ESC colony. Moreover, if ES cells are allowed to clump together to form embryoid bodies, they begin to differentiate spontaneously. Although spontaneous differentiation is a good indication that a culture of ES cells is healthy, it is not an efficient way to produce specific cell types. Generation of specific types of differentiated cells, such as heart muscle cells, blood cells, or nerve cells, from embryonic stem cells, can be achieved by ways including activating endogenous transcription factors; transfection of ESCs with ubiquitously expressing transcription factors; exposure of ESCs to selected growth factors; or coculture of ESCs with cell types capable of lineage induction. ESCs may be induced to form the lineage of interest by a combination of growth factors and/or their antagonists (Loebel D A, et. al. Dev Biol 264:1-14, 2003).

One embodiment of the invention includes neural stem cell derivatives. Formation of ectodermal derivatives is very common in spontaneously differentiating hESCs and is commonly considered a developmental default pathway. The neural differentiating pathway can be enhanced in cultures (Carpenter M K, et. al. Exp Neurol 172:383-397, 2001). The directed differentiation of hESCs into neuroectoderm may be efficiently achieved using Noggin, which is an antagonist to BMP signaling that is involved in the paracrine loop that drives hESCs into flattened epithelial that express genes characteristic of extra-embryonic endoderm. The Noggin-induced cultures are capable of renewal as relatively homogeneous colonies of neuroectoderm. Methods of inducing hESCs to differentiate into other specific cell types including epithelial cells, differentiated cardiomyocytes, cells of the respiratory lineage, keratinocytes, cells of the hematopoietic lineage, cells expressing markers of hepatocytes, and pancreatic islet-like clusters have been reported in the relevant art (Trounson A. Endocrine Reviews 27 (2): 208-219, 2006). If ES cells can be reliably induced to differentiate into specific cell types, they may be used to treat certain diseases in the future. Diseases that might be treated by transplanting cells generated from human embryonic stem cells include but are not limited to Parkinson's disease, diabetes, traumatic spinal cord injury, Purkinje cell degeneration, Duchenne's muscular dystrophy, heart disease, and vision and hearing loss.

In one embodiment, the present invention provides a method of inducing neural stem cells (NSC) from hESC in defined xeno-free adherent cultures. One of the first tissues to form in the developing embryo is the anlage of the nervous system—neural tube. Three general approaches have been used to generate neural derivatives from ESC: 1) mimicking microenviroment of embryonic development by forming embryoid body intermediates, 2) modeling “neural stem cell niche” by co-culture with stromal cells, which would supply permissive or instructive signals, and 3) inducing default mechanism of neural conversion by depriving ESC of both cell-cell interactions and other self-renewal stimuli from their microenvironment (Cai et al. Dev Dyn, 2007). Efficient neural conversion of hESC under xeno-free conditions has not been reported yet. The present invention provides a highly efficient direct method for committing hESC toward neural cell lineage under xeno-free conditions. This approach avoids phenotypic heterogeneity, which arises within microenvironment of neurospheres and converts hESC directly into neural stem cells (NSC) under fully defined adherent culture conditions. The method of the present invention demonstrates that removal of cell-cell contacts, defined extracellular matrix (ECM) and defined serum-free neural stem cell medium are sufficient for converting hESC into primitive NSC expressing both early neural lineage (e.g. nestin) and pluripotency markers (e.g. Oct 3/4). Slightly altered defined culture conditions lead to differentiation of these cells into mature neurons, oligodendrocytes, and astrocytes.

### IV. Stem Cell Markers

The present invention embodies characterization of stem cells based on cell phenotypes including cell surface markers. Stem cells can be isolated based on a distinctive set of cell surface markers. For ESCs, several markers are particularly important. Oct-4 (also termed Oct-3 or Oct3/4), one of the POU transcription factors, is expressed in totipotent embryonic stem and germ cells. A critical level of Oct-4 expression is required to sustain stem cell self-renewal and pluripotency (Niwa, H. et al. (2000) Nat. Genet. 24:372). Differentiation of embryonic stem (ES) cells results in down-regulation of Oct-4, an event essential for a proper and divergent developmental program. Oct-4 is not only a master regulator of pluripotency that controls lineage commitment, but is also the first and most recognized marker used for the identification of ES cells. The second class of markers includes stage specific embryonic antigens (SSEAs). SSEA-1 is expressed on the surface of preimplantation-stage murine embryos and has been found on the surface of teratocarcinoma stem cells, but not on their differentiated derivatives. SSEA-3 and -4 are synthesized during oogenesis and are present in the membranes of oocytes, zygotes and early cleavage-stage embryos. Biological roles of these carbohydrate-associated molecules have been suggested in controlling cell surface interactions during development. Undifferentiated primate ES cells, human EC and ES cells express SSEA-3 and SSEA-4, but not SSEA-1. Undifferentiated mouse ES cells express SSEA-1, but not SSEA-3 or SSEA-4 (Thomson, J. A. et al. (1998) Science 282:1145; Thomson, J. A. et al. (1998) Curr. Top. Dev. Biol. 38:133).

In one aspect, the present invention provides a method of diagnosing cancer or detecting a cancerous or pre-cancerous cell comprising obtaining a cell from a subject, for example, cancer or tumor cell or progenitor cell or tumor tissue can be obtained from the subject; assessing molecular and/or cellular profile of the stem cell from the subject; and comparing the profile of the stem cell from the subject with a database of multi-stage stem cell carcinogenesis of the present invention. Stem cells can be isolated based on any stem cell marker disclosed herein and various methods of isolating stem cells from a subject, for example, a human, are known in the relevant field.

For neural stem cells (NSC), nestin is a class VI intermediate filament protein expressed predominantly in stem cells of the central nervous system (CNS) (Frederiksen, K. et al. (1988) J. Neurosci. 8:1144). It is not expressed on nearly all mature CNS cells. Nestin has been the most extensively used marker to identify CNS stem cells within various areas of the developing nervous system and in cultured cells in vitro. Its transient expression has been suggested to be a major step in the neural differentiation pathway. PSA-NCAM (Polysialic acid-neural cell adhesion molecule) is the embryonic form of NCAM. It is highly polysialylated and is mainly expressed in the developing nervous system. In the adult, PSA-NCAM expression is restricted to regions that retain plasticity. A neuronal-restricted precursor identified by its high expression of PSA-NCAM can undergo self-renewal and differentiate into multiple neuronal phenotypes. Finally, p75 Neurotrophin R (NTR), also known as low affinity nerve growth factor (NGF) receptor, binds to NGF, BDNF, NT-3 and NT-4 equally with low affinity. p75NTR, when activated in the presence of Trk, enhances responses to neurotrophin. TrkC receptors working together with p75 NTR have been suggested to serve critical functions during the development of the nervous system. Neural crest stem cells (NCSCs) have been isolated based on their surface expression of p75NTR (Stemple, D. L. et al. (1992) Cell 71:973. Morrison, S. J. et al. (1999) Cell 96:737). Freshly isolated p75NTR⁺ NCSCs from peripheral nerve tissues can self-renew and generate neurons and glia both in vitro and in vivo. In addition, neuroepithelial-derived p75NTR⁺ cells are also able to differentiate into neurons, smooth muscle and Schwann cells in culture. Recently, p75 NTR has been used as a marker to identify mesenchymal precursors as well as hepatic stellate cells. Sox-1 is one of the major markers for neural stem cells (NSC).

In addition to the aforementioned stem cell markers, molecules that have been commonly used to identify, characterize, and/or isolate stem cells include, but are not limited to, alkaline phosphatase, CCR7, CD9, Thy1, CD38, CD30, CD43, CD48, CD90, CD105, CD117/c-kit, CD123, CD133, ABCB5, CD135/Flk2, CD144 (VE-cadherin), CD150, CD338/ABCG2, c-Met, Foxd3, GCTM-2, genesis, germ cell nuclear factor, hepatocyte nuclear factor-4 (HNF-4), keratan sulfate antigens, neuronal cell adhesion molecule (N-CAM), Nanog, Notch-1, Pax6, Rex1, SOX2, Stem cell factor (SCF or c-kit ligand), telomerase, Tra1-60, Tra1-81, UTF1 transcription factors, TERF1, CHK2, DNMT3 DNA modifiers, GFA1 surface markers, GDF3 growth factor, TDGF1 receptor, Stella, FLJ10713, TG-30, TG-343, and vimentin.

### V. Cancer Stem Cells/Tumor Initiating Cells

Several aspects of the present invention involve cancer stem cells including the generation of cancer stem cells or tumor initiating cells from non-cancer stem cells. Methods and systems of the present invention as described herein are applicable to both tumor initiating cells (TIC) and cancer stem cells (CSC). TIC and CSC are used interchangeably herein. Cancer stem cells (CSCs) are a sub-population of cancer cells found within tumors or hematological cancers that possess characteristics normally associated with stem cells. CSCs are believed to be tumorigenic, in contrast to the bulk of cancer cells, which are thought to be non-tumorigenic. CSCs have stem cell properties such as self-renewal and the ability to differentiate into multiple cell types. CSCs are also capable of forming heterogeneous tumors in immunodeficient mice at high frequency. It has been suggested that CSCs persist in tumors as a distinct population and cause relapse and metastasis by giving rise to new tumors. Most human tumors have now been shown to contain a sub-population of cells that display cancer stem cell characteristics. The types of cancer include but are not limited to leukemia, breast cancer, melanoma, lung cancer, brain cancers, colon cancers, pancreatic cancer, and ovarian cancer. Development of specific therapies targeted at cancer stem cells holds hope for improvement of survival and quality of life of cancer patients, especially for patients having metastasis.

The origin of cancer stem cells has not been clearly elucidated in the art. It is very likely that a mutation from a normal stem cell can produce a cancer stem cell. Also, in tissues with a high rate of cell turnover, such as the skin or gastrointestinal epithelium, it is possible that stem cells are the only cells that live long enough to acquire enough genetic abnormalities to become cancerous. Spontaneous transformation of human nontumorigenic stem cells to highly transformed and tumorigenic cancer stem cells has been demonstrated in the formation of human gliobastoma (Shiras, et. al. Stem cells Express, 2007). However it is still possible that more differentiated cancer cells could acquire properties of a stem cell.

The existence of cancer stem cells has several implications in terms of cancer treatment and therapies. Normal stem cells are naturally resistant to chemotherapeutic agents because they have various pumps (such as MDR) that pump out drugs. Stem cells also have DNA repair proteins and a slow rate of cell turnover. Cancer stem cells, being the mutated counterparts of normal stem cells, may also have similar functions which allow them to survive various therapies. By selectively targeting cancer stem cells, it would be possible to treat patients with aggressive tumors, as well as preventing the tumor from metastasizing.

Cancer stem cells share many aspects of biology with normal stem cells (i.e. unaltered and untransformed stem cells). First, both normal stem cells and cancer stem cells undergo self-renewal, and emerging evidence suggests that similar molecular mechanisms regulate self-renewal in normal stem cells and their malignant counterparts (Boman, B. M., and M. S. Wicha 2008 J Clin Oncol. 26:2795-9; Dreesen, O. and A. H. Brivanlou 2007 Stem Cell Rev 3: 7-17; Postovit, L. M., et. al. 2007 J Cell Biochem 101: 908-17; Serakinci, N., and C. Erzik. 2007 Regen Med. 2:957-65; Sole, R. V., C. Rodriguez-Caso, T. S. Deisboeck, and J. Saldana 2008 J Theor Biol.; Wang, Y., and S. A. Armstrong 2008 Cell Stem Cell. 2:297-9; Wong, D. J., et. al. 2008 Cell Stem Cell. 2:333-44; Zhang, H. and Z. Z. Wang (2008) J Cell Biochem 103: 709-18). Second, it is quite likely that mutations accumulate in normal stem cells and eventually lead to cancer. Finally, it is likely that tumors contain a cancer stem cell population with indefinite proliferative potential that drives the growth and metastasis of tumors (Southam & Brunschwig, 1961, Cancer 14:971-78; Bruce & Gaag, 1963, Nature 199:79-80; Wodinsky et al., 1967, Cancer Chemother. Rep. 51:415-21; Bergsagel & Valeriote, 1968, Cancer Res. 28:2187-96; Park et al., 1971, J. Natl. Cancer Inst. 46:411-22; Hamburger & Salmon, 1977, Science 197:461-3; Lagasse & Weissman, 1994, J. Exp. Med. 179:1047-52; Reya et al., 2001, Nature 414:105-11; Al-Hajj et al., 2002, PNAS 100:3983).

Cancer stem cells and normal stem cells also express many genes in common (Müller F J et. al. Nature 2008 Sep. 18; 455(7211):401-5). A number of genes classically associated with cancer can also regulate normal stem cell development (reviewed in Reya et al., 2001, Nature 414:105-11 and Taipale & Beachy, 2001, Nature 411:349-54). Microarray analysis has identified more than 300 genes that are highly expressed in common in hESCs and human embryonal carcinoma cells and seminomas (Sperger J. Proc Natl Acad Sci USA. 2003 Nov. 11; 100(23): 13350-13355). Among the 330 genes identified as highly expressed in both ESCs and CSCs includes POU5F1 (Oct-4), a transcription factor that has been previously shown to be expressed only in the pluripotent cells of the embryo and to promote differentiation when down-regulated. Since POU5F1 is a central regulator of pluripotency, genes having a similar expression pattern may also be central to maintaining pluripotent cells. The gene that clusters closest to POU5F1 is FLJ10713, among the top five genes in hESCs and human embryonal carcinoma cells. A homolog of FLJ10713 has been shown to be highly expressed in mouse ES cells. Among those genes only highly expressed in hESCs and human embryonal carcinoma cells also include a DNA methylase (DNMT3B), which functions in early embryogenesis, and Foxd3, a forkhead family transcription factor that interacts with Oct4, essential for the maintenance of mouse primitive ectoderm. Sox2 is also highly expressed and is known to be important in pluripotentiality.

### VI. Mutagenesis:

Embodiments of the present invention include generation of cancer stem cells or tumor initiating cells from non-cancer stem cells via mutagenesis. Mutation refers to a change in the genetic material. Mutations may be gross at the level of the chromosome or point alterations. The latter can involve just a single nucleotide pair in DNA. There are two major types of point mutations: base pair (nucleotide pair) substitutions and frameshift mutations. The consequences of base substitution mutations in protein coding regions of a gene depend on the substitution and its location. They may be silent, not resulting in a new amino acid in the protein sequence. They could also result in an amino acid substitution referred to as a missense mutation. Base substitutions in a protein coding region may also mutate an amino acid codon to a termination codon or vice versa. Their consequences depend on what they do to the level of expression of the gene product and/or on what amino acid substitution may have occurred and where it is in the protein. The frameshift mutations result from the insertion or deletion of one or more nucleotides in the coding region of a gene. This causes an alteration of the reading frame. A frameshift mutation changes all the amino acids downstream and is very likely to create a nonfunctional product since it may differ greatly from the normal protein. Further, reading frames other than the correct one often contain stop codons which will truncate the mutant protein prematurely.

Mutagenesis is referred to as a process by which the genetic information of an organism is changed in a stable manner, either spontaneously or experimentally by the use of chemicals or radiation. There are several types of mutagenesis: PCR mutagenesis, transposon mutagenesis, site-directed mutagenesis, directed mutagenesis, insertional mutagenesis, and targeted mutagenesis. The concepts and methods of the abovementioned types of mutagenesis are well known in the art.

The present invention embodies a directed mutagenesis approach to generate cancer stem cells at different stages of stem cell carcinogenesis. Directed mutagenesis refers to the hypothesis that organisms can respond to environmental stresses through directing mutations to certain genes or areas of the genome. It is widely accepted that a cancer cell arises via a multi-step progression from a normal cell to an increasingly pre-cancerous cell ultimately evolving into a full-fledged cancer cell with malignant capability. This process is driven by neo-Darwinian evolution whereby those increasingly precancerous cells with the most advantageous mutations (e.g., growth-promoting mutations) undergo natural selection and expansion over their less “fit” competitors. In this manner, multiple rounds of mutation followed by selection ultimately lead to a malignant cancer cell which is both highly proliferative and highly mutated. Cancer development is associated with DNA replication stress, which leads to genomic instability and selective pressure for inactivation of tumor suppressor genes such as p53 (Gorgoulis V G et. al., Nature 2005, 434(7035):907-913). Previous studies have shown that an extended in vitro culture of human immortalized cells in tissue culture conditions that generate DNA damage and create selective pressure for inactivation of tumor suppressor genes such as p53 can result in tumorigenic cell transformation (Chen W D et. al. Journal of the National Cancer Institute 2000 92(6):480-485).

One embodiment includes exposing non-cancer stem cells or their derivatives to a mutagen. A mutagen is a natural or human-made agent, which can alter the structure or sequence of DNA. There are two main types of mutagens: chemical mutagens and physical mutagens, i.e. radiation. The chemical mutagens generally include base analogs, chemicals which alter structure and pairing properties of bases, intercalating agents, and agents altering DNA structure. Physical mutagens, i.e. radiation, include ionizing radiation and non-ionizing radiation, e.g. UV. Among the chemical mutagens, the base analogs structurally resemble purines and pyrimidines and may be incorporated into DNA in place of the normal bases during DNA replication. Nitrous acid, nitrosoguanidine, methyl methanesulfonate, and ethyl methanesulfonate are all chemical mutagens that alter the structure of the bases, e.g. adding methyl or ethyl groups. Examples of the intercalating agents include acridine orange, proflavin, and ethidium bromide. They interact with bases of DNA and insert between them, resulting in frameshifts. Mutagens are typically also carcinogens. But not all mutations are caused by mutagens. Spontaneous mutations occur due to errors in DNA replication, repair and recombination. Mutagens that induce mutations which may favor cancer development include, but are not limited to, abrin; 7-Acetamido-1-methyl-4-(para-(para-((1-methylpyridinium-4-L)amino)benzamido)anilino) quinolinium di-para-toluenesulfonate; N-Acetoxy-2-acetamidophenanthrene; 6-Acetoxy-benzo(a)pyrene; Acetoxycycloheximide; 1,-Acetoxyestragole; n-Acetoxy-n-acetyl-2-aminofluorene; N-Acetoxy-N-acetyl-2-aminofluorene; Acetyl hydrazide; 4-(para-(para-Acetylbenzamido)anilino)-6-amino-1-methylquinolinium)-para-amidinohydrazone-para-toluenesulfonate-mon-para-toluenesulfonate; N-Acetylbenzidine; N-Acetyl-4-biphenylhydroxylamine; 1-Acetyl-2-isonicotinoylhydrazine; 6-Acetyloxymethylbenzo(a) pyrene; 5-(1-Acetyloxy-2-propenyl)-1,3-benzodioxole; Acetylsalicylic acid; Aclacinomycin Y; Acridine; Acridine hydrochloride; 4,-(9-Acridinylamino)-2,-methoxymethanesulfonanilide; 4,-(9-Acridinylamino)-3,-methoxymethanesulfonanilide; 4,-(9-Acridinylamino) methanesulphon-meta-anisidide; 4,-(9-Acridinylamino)methanesulfon-meta-aniside monohydrochloride; Acronycine; Actinomycin C; Actinomycin D; Actinomycin S3; Adenosine; Adenosine-3,-(alpha-amino-para-methoxyhydrocinnamamido)-3,-deoxy-n,n-dimethyl; Adrenalin bitartrate; Adriamycin-14-octanoatehydrochloride; Aflatoxin B2; Aflatoxin B1-2,3-dichloride; Aflatoxin M1; Ageratochromene; Aizen malachite green; 4-n-d-Alanyl-2,4-diamino-2,4-dideoxy-1-arabinose; Aldocortene; 1-Allyl-2,5-dimethoxy-3,4-methylenedioxybenzene; 4-Allyl-1,2-dimethoxybenzene; Aluminon; Aluminum chloride; Aluminum chloride hexahydrate; Amikhellin hydrochloride; 4-((3-Amino-4-((4-((1-methylpyridinium-4-YL)amino)benzoyl)amino)phenyl)amino-1-methylquinolinium)dibromide; 6-Amino-4-((3-amino-4-(((4-((1-methylpyridinium-4-YL)amino)phenyl)amino)carbonyl)phenyl)amino)-1-methylquinium), diiodide; 2-Amino-4-(ethylthio)butyric acid; 4-Amino-4,-(2-carbamoylethyl)-1,1-dimethyl-n,4,-bi(pyrrole-2-carboxamide; 5-Amino-1,6-dihydro-7h-v-triazolo(4,5-d)pyrimidin-7-one; 2-Amino-3,4-dimethylimidazo (4,5-f)quinoline; 2-Amino-1,4-naphthoquinone imine hydrochloride; 2-Aminoacridine; Aminoacridine hydrochloride; 1-Aminoanthraquinone; 4-Aminobiphenyl; 2-Amino-2-deoxy-1-ascorbic acid; 2-Amino-6-dimethyl-4-(para-(para-((para-((1-methylpyridinium-3-YL)carbamoyl)phenyl)carbabenzamidolanilino)pyrimidimium), diiodide; 4-Aminodiphenyl; 9-Aminoellipticine; 2-Aminoethanethiol; 6-Amino-1-ethyl-4-(para((para-((1-ethylpyridinium-4-YL)amino)phenyl)carbamoyl)anilinoquinolinium)dibromide; 6-Amino-1-ethyl-4-(para-(para-((1-ethylpyridinium-4-YL)amino)benzamido)anilino)quinolinium diiodide; 6-Amino-1-ethyl-4-para-((para-((1-ethylpyridinium-4-YL)amino)2-aminophenyl; (s)-3-(6-Amino-9h-purin-9-yl)-1,2-propanediol; 3-Amino-5H-pyrido(4,3-b)indole; 6-Amino-8-methoxy-1-methyl-4-(para-(para-((1-methylpyridinium-4-YL)amino)benzamido)anilinoquinolinium) DI-p-toluenesulfonate; 7-Amino-4-(2-methoxy-para-(para-((1-methylpyridinium-4-YL)amino)benzamido)anilino)-1-methylquinolinium)dibromide; 2-Amino-6-methyldipyrido(1,2-a:3,2,-d)imidazole; 3-Amino-1-methyl-5H-pyrido(4,3-b) indole; 2-Amino-3-methylimidazo (4,5-f)quinoline; 2-Amino-4-(5-nitro-2-furyl)thiazole; Aminonucleoside puromycin; 6-Amino-1-propyl-4-(para-((para-((1-propylpyridinium-4-YL)amino)-2-aminophenyl)carbamoyl)anilino)quinolium)diiodide; 6-Amino-1-propyl-4-(para-((para-((1-propylpyridinium-4-YL)amino)phenyl)carbamoyl)anilino)quindinium)dibromide; 2-Aminopurine-6-thiol; 3,3,-(2-Aminoterephthaloybis(imino-para-phenylenecarbonylimino))bis(1-Ethylpyridinium),DI-paratoluenesulfonate; 3,3,-(2-Aminoterephthaloylbis(imino(3-amino-para-phenylene)carbonylimino))bis(1-ethylpyridinium),DI-p-toluenesulfonate; 3,3,-(2-Aminoterephthaloylbis(imino(3-amino-para-phenylene)carbonylimino))bis(1-propylpyridinium),DI-p-toluenesulfonate; 3,3,-(2-Aminoterephthaloylbis(imino-para-phenylenecarbonylimino))bis(1-methylpyridinium),DI-para toluenesulfonate; 5-Aminouracil; Ammonium bromide; Anguidin; Aniline mustard; Anthracene; Anthramycin; anti-Benzo(a)pyrene-7,8-dihydrodiol-9,10-oxide; Antibiotic CC 1065; Antibiotic MA 144A1; Antibiotic MA 144s2; Antimony potassium tartrate; Antipyrine; 9-beta-d-Arabino furanosyl adenine; 1-b-d-Arabinofuranosylcytosine hydrochloride; 1-b-d-Arabinofuranosyl-5-fluorocytosine; Ara-C palmitate; Arecoline; Arecoline hydrochloride; Aroclor 1254; ASTA Z 7557; ATP; Atropine sulfate (1:1); Auramycin A; Auramycin B; Auranofin; Aurantine; Aureine; Auromomycin; Azacytidine; 5-Azadeoxycytidine; Azaleucine; Aziridinylquinone; Azoxymethane; Return to Search KeysB(a)P Epoxide II; Bacilysin; Basic orange 3RN; Baumycin A1; Baumycin A2; Benz (a) anthracene; Benz(a)anthracene-7-methanol; Benz(a)anthracene-5-OL; Benz(a)oxireno(c)anthracene; Benzhydryl; Benzidine; 1,2-Benzisothiazol-3 (2H)-one-1,1-dioxide; Benzo(a)pyrene; Benzo(a)pyrene diol epoxide anti; Benzo(a)pyrene-7,8-dihydrodiol; Benzo(a)pyrene-3,6-dione; Benzo(a)pyrene-4,5-oxide; Benzo(a)pyrene-6-methanol; Benzo(a)pyren-11-OL; Benzo(a)pyren-1-OL; Benzo(a)pyren-2-OL; Benzo(a)pyren-3-OL; Benzo(a)pyren-7-OL; Benzo(a)pyren-9-OL; Benzo(b)triphenylene; Benzo(e)pyrene; Benzo(j)fluoranthene; 1,3-Benzodioxole-5-(2-propen-1-OL); Benzoic acid; Benzoyl peroxide; n-Benzoyloxy-n-methyl-4-aminoazobenzene; Benzyl chloride; N-Benzyl-b-(isonicotinylhydrazino)propionamide; Beryllium; Bestrabucil; Betel quid extract; Betnelan phosphate; BHT (food grade); 4,4, Biacetanilide; 3,3,-(Bicyclo(2.2.2)octane-1,4-diylbis(carbonylimino-4,1-phenylenecarbonylimino))bis(1-ethylpyridinium) salt with 4-methyl benzenesulfonic acid (1:2); Biltricide; 1,4-Bis((2-((2-hydroxyethyl)amino)ethyl)amino)anthraquinone; 4,4,-Bis(1-amino-8-hydroxy-2,4-disulfo-7-naphthylazo)-3,3,bitolyl,; Bis(2-chloroethyl) methylamine hydrochloride; cis-Bis(cyclopentylammine)platinum(II); 2-(N,N-Bis(2-hydroxyethyl)amino)-1,4-benzoquinone; Bisantrene hydrochloride; Bleomycin; Bleomycin A2; Bleomycin PEP; Borane, compound with trimethylamine (1:1); Botryodiplodin; Bouvardin; 7-Bromo methyl-12-methylbenz(a) anthracene; Bromoacetaldehyde; alpha-Bromoacetic acid; 2-Bromoacrolein; Bromochloroacetonitrile; 7-Bromomethylbenz(a)anthracene; 6-Bromomethylbenzo(a)pyrene; Bromotrichloromethane; (e)-5-(2-Bromovinyl)-2′-deoxyuridine; Bruceantin; 1,4-Butanediamine; Butonate; Butyl mesylate; 1-(tert-Butylamino)3-(3-methyl-2-nitrophenoxy)-2-propanol; n-Butyl-(3-carboxy propyl)nitrosamine; 6-Butyl-4-nitoquinoline-1-oxide; Butylnitrosoaminomethyl acetate; n-Butyl-N-nitroso-1-butamine; n-Butyric acid; beta-Butyrolactone; Return to Search KeysC.I. 45405; Cadmium chloride; Cadmium chloride, hydrate (2:5); Cadmium sulfate (1:1); Cadmium sulfide; Cadmium(II) Acetate; Caffeine; Calcium chromate; Calcium cyclohexylsulphamate; Calcium trisodium diethylene triamine pentaacetate; Camptothecine; Cannabidiol; Cannabinol; Capsaicin; Caracemide; n-(Carbamoylmethyl)-2-diazoacetamide; Carboplatin; Carboxycyclophosphamide; 6-Carboxyl-4-nitroquinoline-1-oxide; Caminomycin I; Catechol; CCNU; Chartreusin; Chloramphenicol; n-(2-Chloro ethyl)diethylamine; 2-Chloro-5-(3,5-dimethylpiperidino sulphonyl)benzoic acid; 2-((4-Chloro-6-(2,3-xylidino)-2-pyrimidinyl)thio)-n-(2-hydroxyethyl)acetamide; (4-Chloro-6-(2,3-xylidino)-2-pyrimidinylthio)acetic acid; Chloro(triethylphosphine)gold; 4-Chloro-1,2-diaminobenzene; 1-Chloro-2,4-dinitrobenzene; Chloroacetonitrile; 1-Chloro-2-bromoethane; Chlorocylcine; Chlorodinitrobenzenes; 1-(2-Chloroethyl)-1-nitrosourea; 1-(2-Chloroethyl)-3-(2,6-dioxo-3-piperidyl)-1-nitrosourea; 1-(2-Chloroethyl)-3-(b-d-glucopyranosyl)-1-nitrosourea; 1-(2-Chloroethyl)-3-(trans-4-hydroxycyclohexyl)-1-nitrosourea; n,n-Bis(2-chloroethyl)benzylamine; 1-(2-Chloroethyl)-3-cyclohexyl-1-nitrosourea; n-(2-Chloroethyl)dimethylamine; 1-(2-Chloroethyl)-3-(2-hydroxyethyl)-1-nitrosourea; 1-(2-Chloroethyl)-3-(4-methylcyclohexyl)-1-nitrosourea; 4-(2-Chloroethyl)morpholine; n-(b-Chloroethyl)-n-nitrosoacetamide; 2-Chloroethyl-2-hydroxyethyl sulfide; 2-Chloroethyl-n-nitrosourethane; 5-Chloro-7-iodo-8-quinolinol; Chloromethyl methyl ether; 7-Chloro-1-methyl-4-(para-((1-methylpyridinium-4-YL)amino)phenyl)carbamoyl)anilino)quinolinium dibromide; 6-Chloro-1-methyl-4-(para-((para-((1-methylpyridinium-4 YL)amino)phenyl)carbamoyl)anilino)quinolinium,DI-para-toluenesulfonate; 8-Chloro-1-methyl-4-(para-((para-((1-methylpyridinium-4-YL)amino)phenyl)carbamoyl)anilino)quinolinium)DI-para-toluene sulfonate; 4-Chloromethylbiphenyl; n-(3-Chloro-4-methylphenyl)-n′,n′-dimethylurea; Chloronitrobenzene; 6-Chloro-4-nitroquinole-1-oxide; 3-Chloro-ortho-toluidine; 4-Chloro-ortho-toluidine; 5-Chloro-ortho-toluidine; 6-Chloro-ortho-toluidine; 3-Chloro-para-toluidine; Chlorophenamidine; 3-(p-Chlorophenyl)-1,1-dimethylurea; Chloroquine mustard; Chlorotrimethylstannane; Chlorozotocin; Cholesterol; Choline dichloride; Chromium (III) sulfate (2:3); Chromium trichloride hexahydrate; Chromomycin A3; CI Direct blue 6, tetrasodium salt; Cinnamaldehyde; Cirolemycin; Clavacin; Cobalt (II) sulfide; Cobalt diacetate; Cobalt molybdate; Cobalt(II) chloride; Colchicine; Copper (I) sulfide; Copper (II) chloride (1:2); Copper ascorbate; Copper(II) sulfate (1:1); Copper(II) sulfate pentahydrate (1:1:5); Copper(II) sulfide; Coronene; Corticosterone; Cortisol; Cortisone; Coumarin; 2,3-Cresotic acid; Croton oil; Crotonaldehyde; Crotonyloxymethyl-4,5,6-trihydrooxycyclohex-2-enone; Cyanocycline A; Cyanomorpholinoadriamycin; Cycasin; Cyclic amp dibutyrate; Cyclocytidine hydrochloride; Cyclohexanol; Cycloheximide; Cyclohexylamine; Cytochalasin B; Cytosine riboside; Cytoxal alcohol; Cytoxylamine; Return to Search Keys-2,4-D; Daunomycin; Daunomycinol; DDT; Deferoxamine; Dehydroheliotridine; Dehydroretronecine; 4-Demethoxyadriamycin; 4-Demethoxydaunomycin; 3′-Deoxyadenosine; Deoxyadenosine; Deoxycytidine; 2′-Deoxy-5-fluorouridine; 2′-Deoxyguanosine; 2′-Deoxy-5-iodouridine; 7-Deoxynogalarol; 1-Deoxypyrromycin; 2′-Deoxyuridine; Dextromethadone; a-DFMO; o,o-Di(2-chloroethyl)-o-(3-chloro-4-methylcoumarin-7-YL)phosphate; 2′,4′-Diacetate-dis-nogamycin; Diamidine; 2,4-Diamino-5-(para-(para-((-(2,4-diamino-1-methylpyridmidinium-5-YL)phenyl)carbamoylcinnamido) phenyl)-1-ethyl pyridinidinium), DI-para-toluene sulfonate; 3,6-Diamino-2,7-dimethlacridine hydrochloride; 2,7-Diamino-10-ethyl-9-phenylphenanthridinium bromide; 3,6-Diamino-10-methylacridinium chloride with 3,6-acridinediamine; 3,6-Diaminoacridine sulphate (1:1); 3,6-Diaminoacridinium; 2,4-Diaminoanisole; 2,4-Diaminobenzanilide; 4-((4-(((4-(2,4-Diamino-1-ethylpyrimidinium-5-YL)phenyl)amino)carbonyl)phenyl)amino)-1-ethylquinolinium)diiodide; 4-((4-(((4-(2,4-Diamino-1-propylpyrimidinium-5-YL)phenyl)amino)carbonyl)phenyl)amino)-1-propylquinolinium)diiodide; Diaminopropyltetramethylenediamine; Dianhydrogalactitol; Dianhydromannitol; 2,5-Diazirino-3,6-dipropoxy-para-benzoquinone; 5-Diazoimidazole-4-carboxamide; Dibenz (a,h) anthracene; Dibenz(a,h) anthracene-5-OL; Dibenzamine; 7H-Dibenzo (c,g) carbazole; Dibromdulcitol; Dibromoacetonitrile; 1,2-Dibromo-3-chloropropane; 2,3-Dibromopropanol; Dibutyldichlorostannane; cis-Dichloro(dipyridine)platinum (II); Dichloro(ethylenediammine)platinum (II); Dichloroacetonitrile; 8-Dichloroacetoxy-9-hydroxy-8,9-dihydro-aflatoxin B1; 3,3,-Dichlorobenzidine; 7-((3,4-Dichlorobenzyl)aminoactinomycin D; Dichloromethane; 3-(3,4-Dichlorophenyl)-1-methoxymethylurea; 1,3-Dichloro-2-propanol; Dichlorvos; cis-Dicyclohexylamminedichloroplatinum (II); Didemnin B; 2′,3′-Dideoxyadenosine; Dieldrin; alpha,alpha-Diethyl-(E)-4,4,-stilbenediol bis(dihydrogen phosphate); 1-(2,-Diethylamino)ethylamino-4-methylthioxanthenone; 3-Diethylamino-5H-pyrido(4,3-b)indole; Diethylstilbesterol; Diethylstilboestrol-3,4-oxide; Dihydantoin; 1,4-Dihydrazinophthalazine sulfate; 9,10-Dihydro-9,10-dihydroxybenzo(a)pyrene; 4,5-Dihydro-4,5-dihydroxybenzo(a)pyrene; 7,8-Dihydro-7,8-dihydroxybenzo(a)pyrene-9,10-oxide; Dihydrohelenalin; (+/−)-7,b,8,a-Dihydroxy-9,b,10,b-epoxy-7,8,9,10-tetrahydrobenzo(a) pyrene; (b)-7,a,8,b-Dihydroxy-9,b,10,b-epoxy-7,8,9,10-tetrahydrobenzo(a) pyrene; (−)-cis-7,b,8,a-Dihydroxy-9,b,10,b-epoxy-7,8,9,10-tetrahydrobenzo(a)pyrene; (+,−)-trans-7,8-Dihydroxy-7,8-dihydrobenzo(a)pyrene; 7-b,8-a-Dihydroxy-9-a,10-a-epoxy-7,8,9,10-tetrahydrobenzo(a)pyrene; 3,4-Dihydroxyphenylglyoxime; 3,3′-Dimethoxybenzidine; 4,5,-Dimethyl angelicin; Dimethyl sulfate; 1,1-Dimethyl-3-(a,a,a-trifluoro-m-tolyl) urea; 1,6-Dimethyl-4-(para-((para-((1-methylpyridinium-4-YL)amino)phenyl)carbamoyl)anilino)quinolinium)DI-para-toluenesulfonate; 1,8-Dimethyl-4-(para-((para-((1-methylpyridinium-4-YL)amino)phenyl)carbamoyl)anilino)quinolium)DI-para-toluene sulfonate; 1,3-Dimethyl-4-(para-((para-((1-methylpyridinium-4-YL)amino)phenyl)carbamoyl)anilinoquinolinium), dibromide; 6,7-Dimethyl-1,2-benzanthracene; N,N-Dimethylacetamide; Dimethylamine borane; 10-(2-(Dimethylamino)propyl)phenothiazine; 9-(para-Dimethylaminoanilino) acridine; Dimethylaminoantipyrine; 4-Dimethylaminoazobenzene; 6,12-Dimethylanthanthrene; 7,12-Dimethylbenz(a)anthracene-5,6-oxide; Dimethylbenzanthracene; 7,12-Dimethyl-5-fluorobenz(a)anthracene; 1,1-Dimethylhydrazine; 1,2-Dimethylhydrazine; 1,2-Dimethylhydrazine dihydrochloride; 1,1-Dimethylhydrazine hydrochloride; 3,5-Dimethyl-4-nitropyridine 1-oxide; 2,5-Dimethyl-4-nitropyridine-1-oxide; 1,3-Dimethylnitrosourea; 1,1-Dimethyl-3-phenylurea; 1,3-Dimethylurea; 2,4-Dinitrophenylhydrazine; 2,3-Dinitrotoluene; 2,4-Dinitrotoluene; 2,6-Dinitrotoluene; 3,4-Dinitrotoluene; Dioxane; Diphenylnitrosamine; Dipyrido(1,2-a:3′,2′-d)imidazol-2-amine; Disnogalamycinic acid; Distamycin; Distamycin A hydrochloride; Distamycin A/5; 3,5-Di-tert-butyl-4-hydroxybenzoic acid; Dithiothreitol; Diuron; dl-Methadone; Dopamine; Dopamine hydrochloride; Dopan; Doxycycline; dl-Propanolol; Return to Search KeysEchinomycin; Emetine; Epe; Epichlorohydrin; Epipodophyllotoxin; 7,8-Epoxy-7,8,9,10-tetrahydrobenzo(a)pyrene; 9,10-Epoxy-7,8,9,10-tetrahydrobenzo(a)pyrene; 9,10-Epoxy-9,10,11,12-tetrahydrobenzo(e)pyrene; 5,6-Epoxy-5,6-dihydrodibenz(a,h)anthracene; 1,2-Epoxyethylbenzene; 17-a-Estradiol; Estradiol; Estradiol-3-benzoate; Estrone; Ethinyl estradiol; 17-alpha-Ethinyl-5,10-estrenolone; Ethyl alcohol; 2-(N-Ethyl carbamoyl hydroxymethyl)furan; 1-Ethyl-4-(para-apara-(1-ethylpyridinium-4-YL)phenyl)carbamoyl)anilino)quinolium,dibromide; 1-Ethyl-4-(para-(para-((1-ethylpyridinium-4-YL)amino)benzamido)anilino)quinolinium dibromide; 1-Ethyl-6-(para-(para-((1-ethylquinolinium-6-YL)carbamoyl)benzamido)benzamido)benzam; 1-Ethyl-7-(para-(para-((1-ethylquinolinium-7-YL)carbamoyl)benzamido)benzamido)quinolinium), DI-para-toluene sulfonate; 1-Ethyl-4-(para-(para-((para-((1-ethylpyridinium-4-YL)amino)-2-aminophenyl)carbamoyl)cinnamamide) anilino) pyridinium, dibromide; 1-Ethyl-4-(para-(para-((para-((1-ethylpyridinium-4-YL)amino)phenyl)carbamoyl)anilino)quinoliniumidibromide; 3-Ethylamino-5H-pyrido(4,3-b)indole; Ethylene glycol; Ethylene oxide; Ethylenediaminetetraacetic acid; Ethyl-n-butylnitrosamine; 3-Ethyl-4-nitropyridine-1-oxide; (Ethylnitrosamino)methyl acetate; 1-Ethyl-1-nitrosourea; n-Ethyl-n-methyl-p-(phenylazo)aniline; n-Ethyl-n-nitroso-n′-nitroguanidine; ETP; Return to Search KeysFD&C red no. 3; Ferric chloride hexahydrate; Fisetin; Fluocinolide; 9H-Fluorene; Fluorene-2,7-diamine; N-Fluoren-2-YL acetamide; Fluoride; 7-Fluoro-2-acetamido-fluorene; 1-(1-(3-(para-Fluorobenzoyl)propyl)-4-piperidyl)-2-benzimidazolinone hydrochloride monohydrate; 5-Fluoro-2′-deoxycytidine; 4-Fluoro-dl-phenylalanine; 1-(2-Fluoroethyl)-3-cyclohexyl-1-nitrosourea; 3-Fluorophenylalanine; 5-Fluorouridine; Flutamide; Formaldehyde; Fumaric acid; n-Furfuryladenine; Fusarenone X; Fusariotoxin T 2; Return to Search KeysGallium; Gallium (III) nitrate (1:3); Geldanamycin; Gentamycin; Gentisic acid; Gerontine tetrahydrochloride; Gibberellic acid; Glutathione; Glycerin; Glycols, polyethylene, mono((1,1,3,3-tetramethylbutyl)phenyl)ether; Gossypol; Gossypol acetic acid; Grisofulvin; Return to Search KeysHalciderm; Helenalin; Heliotrine; Heparin; 2,2,3,3,6,6,-Hexachloro-1,1,-biphenyl; 2,2,4,4,5,5,-Hexachloro-1,1,-biphenyl; Hexadecylpyridine bromide; 1,2,3,4,5,6-Hexanehexyl; Histamine; Histidine; Homoveratric acid; Hycanthone methanesulfonate; Hydralazine hydrochloride; Hydrazine; Hydrazobenzene; Hydrocortisone-21-acetate; Hydrogen peroxide, 90%; 4-Hydroperoxyphosphamide; 1,-Hydroxy-2,3,-dehydroestragole; 2-Hydroxyaclacinomycin A; N-Hydroxyadenine; 4-(Hydroxyamino)quinoline-1-oxide; 2-Hydroxyamino-1,4-naphthoquinone; n-Hydroxy-2-aminofluorene; n-Hydroxy-1-aminonaphthalene; 17-b-Hydroxy-5-b-androstan-3-one; 4-Hydroxycyclophosphamide; Hydroxydaunorubicin hydrochloride; 20-Hydroxyecdysone; Hydroxylamine; 2-Hydroxy-3-methyl-1,4-naphthoquinone; 7-Hydroxymethyl-12-methylbenz(alpha)anthracene; N-Hydroxy-N-acetyl-2-aminofluorene; 4-Hydroxy-2-nonenal; 1-Hydroxy-3-n-pentyl-d8-tetrahydrocannabinol; n-Hydroxy-para-phenetidine; ((2-Hydroxypropyl)nitroso)amino)acetone; Hydroxyurea; 3-Hydroxyxanthine; Return to Search Keyslmidazole; Imidazolepyrazole; 4,4,-(Imidocarbonyl)bis(m,n-dimethylamine) monohydrochloride; Indium trichloride; 1H-Indole-3-acetic acid; Indomethacin; Inosine; Iodoacetamide; Iodoacetic acid; Ionox 100; Isocarboxazid; Isocyanic acid-2-chloroethyl ester; Isonicotinic acid amide; Isonicotinic acid hydrazide; 3,3,-(Isophthaloylbis(imino-para-phenylenecarbonylimino))bis(1-ethylpyridinium), DI-para-toluene sulfonate; 9-((3-(Isopropylamino)propyl)amino)-1-nitroacridine dihydrochloride; Isopropylmethanesulfonate; Isoquinaldehyde thiosemicarbazone; Isothiourea; Return to Search KeysKethoxal-bis-thiosemicarbazide; Ketoconazole; Khat leaf extract; Return to Search Keys1,2-Amino-4-(guanidinooxy)butyric acid; Lauric acid, sodium salt; 1-Dihydroxyphenyl-1-alanine; 1-Dopa methyl ester; Lead acetate (II), trihydrate; Lead chloride; Lead chromate, basic; Lead monoxide; Lead-molybdenum chromate; Leurocristine sulfate (1:1); Lithium carbonate (2:1); Lithium chloride; Lithocholic acid; 1-Methadone; 1-Naloxone; 1-Norepinephrine; 1-Tryptophan; Luteoskyrin; Lycopersin; Lycorcidinol; Return to Search KeysM-4212; M-12210; MA144 MI; Macbecin II; Macromomycin; Magnesium chloride; Maleic acid-n-ethylimide; Manganese(II) sulfate tetrahydrate (1:1:4); Mannomustine; Mannomustine dihydrochloride; Marasmic acid; Marcellomycin; Maytansine; Mazindol; m-Cresol; Mebanazine oxalate; Medroxyprogesterone acetate; Melipan; Menadione sodium hydrogen sulfite; beta-Mercaptoethylamine disulfide; Mercury methylchloride; meta-(3-Pentyl)phenyl-n-methyl-n-nitrosocarbamate; Methacrylic acid, butyl ester, polymer with 2-(dimethylamino) ethyl methacrylate and methyl methacrylate; 5-Methoxy psoralen; Methoxyacetic acid; 2-Methoxy-6-chloro-9-(3-(2-chloroethyl)aminopropylamino)acridine dihydrochloride; 4-Methoxy-meta-phenylenediamine; 8-Methoxy-1-methyl-4-(para-((para-((1-Methylpyridinium-4-YL)amino)phenyl Carbamoyl)anilino)quinolinium), dibromide; 6-Methoxy-1-Methyl-4-(para-apara-((1-methylpyridinium-4-YL)amino)phenyl)carbamoyl)anilino) quinolinium, DI-para-toluenesulfonate; (+)-2-(Methoxy-2-naphthyl)-propionic acid; Methyl alcohol; Methyl azoxymethyl acetate; Methyl Botryodiplodin; Methyl hydrazine; Methyl marasmate; 1-Methyl-4-((4-(3-((4-((1-methylpyridinium-4-YL)amino)phenyl)amino)3-oxo-1-propenyl)phenyl)amino)quinolinium, dibromide; 1-Methyl-6-((para-(para-((1-methylquinolinium-6-YL)carbamoyl)benzamido)phen; 1-Methyl-4-(para-((para-((1-methylpyridinium-4-YL)amino)phenyl)carbamoyl)anilino)7-nitroquinolinium),DI-para-toluene sulfate; 1-Methyl-4-(para-((para-((1-methylpyridinium-4-YL)amino)phenyl)carbamoyl)anilino) quinolium), dibromide; 1-Methyl-4-(para-((para-(1-methylpyridinium-4-YL)phenyl)carbamoyl)anilino)quinolinium),di-para-toluene sulfonate; 1-Methyl-4-(para-(para-((1-methylpyridinium-4-YL)amino)benzamido)anilino)quinolinium)DI para-toluene sulfonate; 1-Methyl-4-(para-(para-((1-methylpyridinium-4-YL)amino)styryl)anilino)quinolinium), dibromide; 1-Methyl-6-(para-(para-((1-methylquinolinium-6-YL)carbamoyl)benzamido) benzamidolquinolinium), DI-para-toluene sulfonate; 10-Methyl-1,2-benzanthracene; 5-Methyl-3,4-benzpyrene; 11-Methyl-15,16-dihydro-17-oxocyclopenta(a)phenanthrene; 2-Methyl-1,4-naphthoquinone; 4-Methylaminoacetocatechol; 9-Methylanthracene; 2-Methyl-4-bromoaniline; Methylchlortetracycline; 5-Methylcholanthrene; 3-Methylcholanthrene-11,12-oxide; 5-Methylchrysene; Methyl-CNNU; 3-Methyl-4-dimethylaminoazobenzene; Methylene chloride; Methylene dimethanesulfonate; 4,4,-Methylenedianiline; 1-Methyl-9h-pyrido(3,4-b)indole; N-Methylisatin-3-(thiosemicarbazone); Methylmercury hydroxide; n-Methylmitomycin C; N-methyl-N,-nitro-n-n-nitrosoguanidine; n-Methyl-n,p-dinitrosoaniline; 5-Methyl-3-(5-nitro-2-furfyl)pyrazole; 2-Methyl-4-nitropyridine-1-oxide; 3-Methyl-4-nitropyridine-1-oxide; 3-Methyl-4-nitroquinoline-1-oxide; 6-Methyl-4-nitroquinoline-1-oxide; 7-Methyl-4-nitroquinoline-1-oxide; 4-(N-Methyl-N-nitrosamino)-1-(3-pyridyl)-1-butanone; n-Methyl-n-nitroso-4-(phenylazo)aniline; N-Methyl-N-nitrosoaniline; N-Methyl-N-nitrosoethylcarbamate; 4-o-Methyl-12-o-tetradecanoylphorbol-13-acetate; N-Methyl-para-(phenylazo) aniline; Methylthioinosine; 5-(3-Methyl-1-triazeno)imidazole-4-carboxamide; Metoxuron; Minocycline; Mithramycin; Mitomycin B; Mitonafide; Mitoxantrone hydrochloride; Mitozolomide; MNCO; Modeccin toxin; Mogalarol; 3′-Morpholino-3′-deaminodaunorubicin; Muconmycin A; Mycophenolic acid; Myristicin; Return to Search KeysNafoxidine; Naphthacene; beta-Naphthylamine; 2-Naphthylhydroxylamine; Neocarzinostatin; Niacinamide; Nickel sulfide; Nickel(II) carbonate (1:1); Nicotine; 1-Nitro-9-(diethylaminoethylamine)-acridine dihydrochloride; 2,2′-((7-Nitro-4-benzofurazazanyl)imino)bisethanol oxide; 4-Nitro-7-chlorobenzofurazan; 1-Nitro-9-(2-dihydroxyethylamino-ethylamino)-acridine hydrochloride; 1-Nitro-9-(5-dimethylaminopentylamino)-acridine dihydrochloride; Nitrofurantoin; Nitrofurazone; Nitrogen oxide; N-Nitromethylamine; 4-Nitrophenol; 4-Nitropyridine-N-oxide; 1-Nitroso-5,6-dihydrothymine; 1-Nitroso-5,6-dihydrouracil; Nitrosoaldicarb; Nitrosocarbofuran; n-Nitrosodiallyl amine; Nitrosoisopropanolurea; N-Nitrosomethylethanolamine; N-Nitrosomethyl-2-oxopropylamine; Nitroso-2-methylthiopropionaldehyde-ortho-methyl carbamoyl-oxime; N,-Nitrosonornicotine; 1-Nitrosopyrene; N-Nitrosopyrrolidine; Nitrosotrimethylphenyl-N-methylcarbamate; Nivalenol; Nogalamycin; Nogamycin; 19-Norethisterone; Norharman; Novadex; Novobiocin, monosodium salt; Novoembichin; Return to Search KeysOfloxacin; Olivomycin; Oraflex; Orotic acid; ortho-Anisidine; ortho-Dianisidine; 3-(ortho-Fluorophenyl)alanine; ortho-Methyl nogalarol; Ouabain; 1-Oxiranylpyrene; 4,4,-Oxydianiline; Ozone; Return to Search KeysPactamycin; Palladium(2+) chloride; para-Allylanisole; para-Aminobenzoic acid; para-Nitrochlorobenzene; Penicillic acid; Penicillium roqueforti toxin; Pentacene; 1,5-Pentanediamine; Pentostatin; Pentoxyphylline; Pentylcannabichromene; Perfluoroheptanecarboxylic acid; Persantin; Phalloidin; 1,10-Phenanthroline; N-2-Phenanthrylacetohydroxamic acid; Phenetidine; Phenoxymethylpenicillin; 2-Phenylchromone; 1,1,-(para-Phenylenebis(vinylene-para-phenylene))bis(pyridinium) di-para-toluenesulfonate; Phenylhydrazine; Phenyl-para-benzoquinone; 1-Phenylthiosemicarbazide; Phomopsin; Phorbol myristate acetate; Phosphonacetyl-1-aspartic acid; Photodyn; Picryl chloride; 4,4,-(Pimeloylbis(imino-para-phenyleneimino))bis(1-ethylpyridinium) diperchlorate; 4,4,-(Pimeloylbis(imino-para-phenyleneimino))bis(1-methylpyridinium) dibromide; 2,6-piperazinedione-4,4′-propylene dioxopiperazine; Pipram; Platinum chloride; Podophyllotoxin; Poly(1-(2-oxo-1-pyrrolidinyl)ethylene)iodine complex; Poly(oxyethylene)-p-tert-octylphenyl ether; Polychlorinated biphenyl; Polyinosinic:polycytidylic acid copolymer; Polysorbate 80; Potassium chromate (VI); Potassium cyanide (K(Cn)); Predonin; Procarbazine hydrochloride; Progesterone; Propanedial; 1,2-Propanediol; 1,3-Propanediol; Propidium diiodide; Propoxur nitroso; n-Propyl methanesulfonate; 1-Propyl theobromine; 1-Propyl-6-((p-(p-((1-propylquinolinium-6-YL)carbamoyl)benzamido)benzamido)quinlinium),di-para-toluenesulfonate; 6-Propyl-2-thiouracil; Prostaglandin D2; Prostaglandin E1; Protocatechuic acid; Pseudomonas aeruginosa endotoxin; Pseudomonas pseudomallei exotoxin; Purine; Purine-6-thiol; Puromycin chlorohydrate; Pyrazoloadenine; Pyridinium,4,4-(para-phenylenebis(acryloylimino-para-phenyleneimno))bis(1-ethyl-,DI-para-toluene sulfonate); Pyridinium, 3,3,-(2-methoxyterephthaloylbis(imino-para-phenylenecarbonylimino))bis(1-ethyl)-,di-para-toluene sulfonate; Pyridinium, 4,4,-(suberoylbis(imino-para-phenyleneimino))bis(1-methyl-,),dibromide; Pyridinium, 3,3,-(terephthaloylbis(imino(3-chloro-para-phenylene)carbonylimino))bis(1-methyl-DI-para-toluene sulfonate); Pyridinium, chloroterephthaloylbis(imino-para-phenylenecarbonylimino))bis(1-ethyl)-,DI-para-toluene sulfonate; Pyridinium, 3,3,-(2-chloroterephthaloylbis(imino-para-phenylenecarbonylimino))bis(1-methyl-,DI-para-toluene sulfonate); Pyridinium, 3,3,-(2-methoxyterephthaloylbis(imino-para-phenylenecarbonylimino))bis(1-methyl)-,di-para-toluene sulfonate; Pyridinium, 3,3,-(2-methoxyterephthaloylbis(imino-para-phenylenecarbonylimino))bis(1-propyl)-,di-para-toluene sulfonate; Pyridinium, 1-methyl-3-(meta-(para-((para-((1-methylpyridinium-3-YL)carbamoyl) 1,4-naphthyl)carbamoyl)benzamido)benzamido)-,DI-para-toluene sulfonate; Pyridinium, 1-methyl-4-(para-(para-apara-((1-methylpyridinium-4-YL)amino)phenyl)carbamoyl)cinnamido) anilino)-,DI-para-toluene sulfonate; Pyridinium, 1-methyl-3-(para-(para-((para-(1-methylpyridinium-3-YL)phenyl)carbamoyl)phenyl)carbamoyl)benzamido, diiodide; Pyridinium, 3,3,-(2-methylterephthaloylbis(imino-paraphenylenecarbonylimino))bis(1-ethyl)-DI-para-toluene sulfonate; Pyridinium, 3,3,-(2-methylterephthaloylbis(imino-para-phenylenecarbonylimino))bis(1-methyl)-DI-para-toluene sulfonate; Pyridinium, 1-propyl-3-(para-(para-((para-((1-propylpyridinium-3-YL)carbamoyl) cinnamidolphenyl)-, DI-para-toluene sulfonate; Pyridinium,3,3,-(bicyclo(2.2.2)octane-1,4-diylbis(carbonylimino-4,1-phenylenecarbonylino))bis-propyl-,salts w/4-methylbenzenesulfonic acid (1:2); Pyridinium,3,3,-(terephthaloylbis(imino(3-methoxy-para-phenylene)carbonylimino))bis(1-ethyl-,DI-para-toluene sulfonate); Pyridinium,3,3,-(2-aminoterephthaloylbis(imino-para phenylenecarbonylimino))bis(1-propyl)-,DI-para-toluenesulfonate; Pyridinium,1-ethyl-3-(para-(para-((para-(1-ethylpyridinium-3-YL)phenyl)carbamyl)phenylocarbamoyl)cinnamamido)benzamido)-DI-para-toluene sulfonate; Pyridinium,1-methyl-3-(para-(para-apara-((1-methylpyridinium-4-YL)amino)phenyl)carbamoyl)benzamido) benzamido)-,DI-para-toluene sulfonate; Pyridinium,1-methyl-3-(para(para-((para-((1-methylpyridinium-3-YL)amino)phenyl)carbamoyl)benzamido) benzamido)benzamido)-,diiodide; Pyridinium,1-methyl-3-(para-(para-((para-(1-methylpyridinium-3-YL)phenyl)carbamoyl)benzamido)benzamido)-DI-para-toluene sulfonate; Pyridinium,1-propyl-3-(para-(para-((para-(1-propylpyridinium-3-YL)phenyl)carbamoyl)cinnamido)phenyl)-, DI-para-toluene sulfonate; Pyridinium,3,3,-terephthaloylbis(imino-paraphenylenecarbonylimino))bis(1-propyl)-,DI-para-toluene sulfonate; N-(5H-Pyrido(4,3-b)indol-3-YL)acetamide; Pyrimidine; 7-(((2-Pyrrolyl)methyl)amino)-actinomycin D; Pyruvaldehyde; Return to Search KeysQuelamycin; Quercetin; Questiomycin A; Quinine; Quinolinium dibromide; 8-Quinolinol; Return to Search KeysRapamycin; Razoxane; RFCNU; Rhodamine 6G extra base; Rhodamine WT; Rhodirubin A; Rhodirubin B; Rhodium dibutyrate; Rhodium(II) acetate; Rhodium(II) propionate; 2-b-d-Ribofuranosyl-as-triazine-3,5(2h,4h)-dione; 1-b-d-Ribofuranosyl-1,2,4-triazole-3-carboxamide; Riboxamide; Ricin; Rifamycin AMP; RPCNY; Rubidazone; Rubratoxin B; Return to Search KeysS-((trans-1,2-Dichlorovinyl)-1-cysteine; Salicyclic acid; Sangivamycin; Semostine; Sibiromycin; Silver(I) nitrate (1:1); Smoke condensate, cigarette; Sodium aminotriacetate; Sodium azide (Na(N3)); Sodium b-4-methoxybenzoyl-b-bromoacrylate; Sodium butyrate; Sodium chloride; Sodium decanoate; Sodium diethyldithiocarbamate; Sodium diethyldithiocarbamate trihydrate; Sodium monoiodoacetate; Sodium mycophenolate; Sodium myristate; Sodium n-octanoate; Sodium palmitate; Sodium para-aminosalicylate; Sodium salicylate; Sodium selenite; Sodium sulfite (2:1); Sorbitan monooleate; Spermidine; Streptonivicin; 4,4,-(Suberoylbis(imno-para-phenyleneimino))bis(1-ethylpyridinium)dibromide; Succinic acid; Sulfur dioxide; Sulfurmycin A; Sulfurmycin B; Superprednol; Synsac; Return to Search KeysTagamet; Talisomycin S10B; Tallysomycin A; Tannic acid; Tauromycetin-III; Tauromycetin-IV; Telocidin B; Tenulin; 5,5,-(Terephthaloylbis(imino-para-phenylene))bis(2,4-diamino-1-ethylpyrimidinium)-DI-para-toluenesulfonate; 5,5,-(Terephthaloylbis(imino-para-phenylene))bis(2,4-diamino-1-methylpyrimidinium)-DI-para-toluenesulfonate; 5,5,-(Terephthaloylbis(imino-para-phenylene))bis(2,4-diamino-1-propylpyrimidinium)-DI-para-toluenesulfonate; 3,3,-(Terephthaloylbis(imino-para-phenylene))bis(1-propylpyridinium-di-para-toluenesulfonate; 4,4,-(Terephthaloylbis(imino-para-phenylene))bis(1-propylpyridinium-di-para-toluenesulfonate; Terreic acid; tert-Butylhydroperoxide; 2-tert-Butyl-p-cresol; Testosterone; Testosterone propionate; Tetrabutylammonium bromide; cis-Tetrachlorodiammine platinum(IV); Tetrachlorohydroquinone; Tetraethyl ammonium bromide; 3,5,3,5,-Tetrafluorodiethylstilbestrol; 1,2,3,4-Tetrahydro-7,12-dimethylbenz(a)anthracene; Tetrahydro-2-methyl-6-(tetrahydro-2,5-dioxo-3-furyl)pyran-3,4-dicarboxylic anhydride polymer; Tetramethylammonium bromide; 1,1,3,3-Tetramethylurea; Tetrandrine; Tetrapentylammonium bromide; Tetrapropylammonium bromide; Tetrocarcin A; Thalicarpine; Thallium(I) carbonate (2:1); 2-Thienylalanine; 2-Thiocytosine; Thiosemicarbazide; Thymidine; Tilorone hydrochloride; Tin(II) chloride (1:2); Tin(II) chloride dihydrate (1:2:2); Toluene-2,4-diamine; Toluene-2,5-diamine; Toluene-2,6-diamine; Toluene-3,4-diamine; p-Toluidine; p-(p-Tolylazo)-aniline; Tomaymycin; trans-4,-Styrlacetanilide; trans-4,5-Dihydro-4,5-dihydroxybenzo(a)pyrene; (+/−)-trans-7,8-Dihydroxy-9,10-epoxy-7,8,9,10-tetrahydro-benzo(a) pyrene; trans-10,11-Dihydrodibenz(a,e)aceanthrylene-10,11-diol; trans-Benz (a,e) fluoroanthene-12,13-dihydrodiol; trans-Benz (a,e) fluoroanthene-3,4-dihydrodiol; trans-Dichlorodiammineplatinum (II); 1-trans-d8-Tetrahydrocannabinol; 1-trans-d9-Tetrahydrocannabinol; trans-N-(para-Styrylphenyl)acetohydroxamic acid; trans-Tetrachlorodiammine platinum (IV); trans-Vitamin A aldehyde; Triazinate; Trichloroacetonitrile; Triciribine; Triethyltin chloride; Trifluoroacetyladriamycin-14-valerate; n-(a,a,a-Trifluoro-m-tolyl)anthranilic acid; 2,n,n-Trimethyl-4-aminoazobenzene; 2,4,5-Trimethylaniline; 2,4,6-Trimethylaniline; Trinitrochlorobenzene; Trioxacarcin C; Trioxifene mesylate; Tripelennamine; Tris; Tris(2-chloroethyl)amine; Tris(2-chloroethyl)ammonium chloride; Trypsin; Turmeric; Return to Search KeysUrethane; beta-Uridine; Urocanic acid; Return to Search KeysVeratric acid; Vincaleukoblastine; 1-Vinthionine; Vitamin A; Vitamin A acetate; Vitamin A acid; Vitamin E; Volidan; Return to Search KeysWarfarin sodium; Return to Search KeysXanthacridine; 2,4-Xylidine; 2,5-Xylidine; 3,4-Xylidine; Return to Search KeysZinc chloride; and Zinc sulfate heptahydrate.

One embodiment further includes exposing normal stem cells to a mutagen that causes frameshift mutation. Mutagens that induce frameshift mutations include, but are not limited to, polycyclic carcinogens, the acridine family of frameshift mutagens, 9-aminoacridine, proflavine, acridine yellow and acridine orange, ICR compounds, and MNNG.

Deficiency of DNA mismatch repair is a common feature of cancers exhibiting instability of microsatellite DNA sequences. Cancers microsatellite instability are recognizable by their high rate of spontaneous frameshift mutations within microsatellite sequences, their resistance to killing by cytotoxic agents, and their localization to specific tissues, e.g., the proximal colon and stomach. It has been tested that the mismatch repair deficiency of these cancers would make them vulnerable to environmental or chemical frameshift-inducing agents. Previous studies have demonstrated that frameshift-inducing mutagens can selectively induce mutations in mismatch repair-deficient cells as compared to mismatch repair-competent cells. Moreover, environmental exposures may favor development of cancers with microsatellite instability (Chen W D, et. al. J. of National Cancer Institute, vol 92, 2000).

The role of genomic instability in carcinogenesis is thought to generate variability within the tumor cell population and to facilitate selection of genetic variants that have growth advantages in the tumor environment. Therefore continuous chronic exposure of cells to mutagens at concentrations that induce activation of the DNA damage-response, while still allowing DNA replication and cell proliferation, provides conditions for both, mimicking genomic instability through the increased rate of mutations and selecting genetic variants that acquire tolerance to DNA damage-response activation. It has been demonstrated that the simulation of Darwinian evolution in vitro by exposing non-tumorigenic cells to a mutagen, which generates random mutations, and selecting in tissue-culture conditions that mimic the tumor environment can result in the tumorigenic transformation of non-tumorigenic cells (Zientek-Targosz et. al. Molecular Cancer 7:51, 2008). Furthermore, due to the stochastic nature of mutations, all the transformed cell lines have various degrees of tumorigenicity and have acquired different cancerous properties.

The present invention further embodies the acridine mutagen ICR191 to mimic genomic instability and generate genetic variability within populations of normal untransformed stem cells. This DNA-intercalating chemical produces an insertion of one G:C base-pair in a short poly-G:C repetitive DNA sequence. ICR191-induced frameshift mutations generate premature translation termination codons, which frequently initiate mutant mRNA degradations via a nonsense-mediated mRNA decay (NMD) pathway. Chromosomal changes associated with the transformation upon ICR191 exposure can be identified using array comparative genomic hybridization (aCGH). Specific genes mutated in the cells transformed by ICR191 exposure can be identified using microarray-based analysis of mRNA levels alterations induced by the inhibition of NMD in the transformed as well as in the non-transformed control cells, i.e. gene identification by NMD inhibition (GINI) analysis. Identifying genes in the transformed cells containing either inactivating mutations in both alleles or a mutation in one allele accompanied by the loss of the second allele can indicate potential tumor suppressors. In some embodiments, genes can be specifically altered via shRNA/siRNA silencing or mutant gene expression. The concepts and the uses of the aforementioned techniques and the procedures of performing the techniques are well known in the art.

### VII. Stem Cell Carcinogenesis

The present invention embodies compositions and methods for studying stem cell carcinogenesis including generating cancer stem cells from untransformed normal stem cells at various stages of stem cell carcinogenesis. Like normal tissues, cancers are maintained by a population of stem-like cells that can both self-renew and differentiate into downstream, non-self-renewing progenitors and mature cells. Defining feature of cancer stem cells (CSC)/tumor initiating cells (TIC) is their unique ability to initiate tumors from a very small number of injected cells and that generated tumors are serially transplantable. Substantial progress has been made in the identification and characterization of stem cells in the mouse and human systems. There is increasing evidence that a variety of cancers may result from transformation of normal stem cells. For example, a breast cancer stem cell population, with the phenotype CD24-CD44+ lineage, was recently identified. As few as 200 cells of this cancer stem cell population were capable of generating tumors in animals, whereas the bulk of the tumor population was tumorigenic only when implanted in high numbers (Dontu G, et. al. Stem cell Reviews, volume 1, 2005, pp. 207-214(8). Similar to their normal counterparts, the cancer stem cells have the ability to self-renew, driving tumorigenicity and possibly relapse and metastasis, and have the ability to differentiate, generating the heterogeneity of the tumors. The concept of cancer as a disease of normal stem cell transformation has profound implications for the development of new strategies for cancer prevention and therapy.

Current cancer therapies target mature cancer cells and thus, often allow survival of the stem cell population contributing to the cancer relapse. CSC share with embryonic stem cells (ESC) multiple characteristics including the capacity for self-renewal by symmetrical cell division, ability to give rise to multiple cell types, indefinite life span due to telomerase activity and abbreviated cell cycle regulation. Published results show that these phenotypic characteristics are achieved in both CSC and ESC by activation of a number of common developmental pathways. However, while activating some of the normal developmental pathways, cancer stem cells also undergo additional epigenetic and genetic changes that render them irresponsive to the regulatory cues from the environment and enable their deregulated growth and transformation giving rise to neoplastic tissues. Understanding molecular events behind this transformation enables design of rational therapies that would allow survival of normal stem cells while targeting cancer stem cell population.

In some aspects, the present invention utilizes a model to mimic a natural process of malignant transformation. In some embodiments, a random chemical mutagenesis process coupled with the phenotype-based clone selection has been designed. Through this process an unbiased selection of cell clones mutations spectrum some of which exhibit specific cancer stem cell/tumor-forming cell characteristics can be achieved. The clones that pass the characterization process will represent the stage-specific nodes of the drug discovery platform of the present invention with starting hESC population serving as an isogenic normal stem cell control. In some embodiments, the platform of the present invention can be used for molecular characterization of these clones and for drug screening to enable development of CSC/TIC targeting therapies. Key cell phenotypes that are associated with malignant transformation include but are not limited to anchorage independent growth, invasiveness, decreased growth factor dependence, and tumor-inducing and invasive capacity in immunocompromised mice. Moreover, CSC/TIC are commonly characterized by the expression of CSC/SC markers, tumorsphere formation capacity, increased cancer drug resistance and the ability to form serially transplantable tumors from a small number of cells. These phenotypes have been chosen to screen and/or characterize the clones that will be included into the drug discovery platform of the present invention. Anchorage independent growth/tumorsphere formation capacity, invasive phenotype and response to a panel of the common cancer drugs were used for the initial screening of the clones. Selected clones were further characterized for their tumorsphere formation capacity and growth factor independence, expression of cancer and stem cell markers, chromosomal abnormalities, proliferation, drug responsiveness for a wider panel of chemotherapeutic reagents, the ability to initiate tumors and serial transplantability of obtained tumors.

Several signaling pathways are important for oncogenesis of stem cells. First of all, Shh signaling has been implicated in the regulation of self-renewal based on the finding that cells highly enriched for human hematopoietic stem cells exhibit increased self-renewal in response to Shh stimulation in vitro (Bhardwaj et al., 2001, Nat. Immunol. 2:172-80). Several other genes related to oncogenesis have been shown to be important for stem cell function. For example, mice deficient for tal-1/SCL, which is involved in some cases of human acute leukemia, lack embryonic hematopoiesis (Shivdasani et al., 1995, Nature 373:432-4) suggesting that it is required initiation of hematopoiesis, or for the decision to form blood cells downstream of embryonic hematopoietic stem cells (HSCs) (Shivdasani et al., 1995, Nature 373:432-4; Porcher et al., 1996, Cell 86:47-57). Members of the Hox family have also been implicated in human leukemia. Enforced expression of HoxB4 can affect stem cell functions (Buske et al., 2002, Blood 100:862-681; Antonchuk & Humphries, 2002, Cell 109:39-45). Another gene is p21^{cip1}, which is one of the major targets of the p53 tumor suppressor gene. Bone marrow from p21^{cip1} deficient mice has a reduced ability to serially reconstitute lethally irradiated recipients. Failure at serial transfer could be attributed to exhaustion of the stem cell pool, loss of telomeres, or loss of transplantability (Cheng et al., 2000, Science 287:1804-8). In mice, bmi-1, a gene that cooperates with c-myc to induce lymphoma (van Lohuizen et al., 1991, Nature 353:353-55; van der Lugt et al., 1994, Genes & Dev. 8:757-69), is required for the maintenance of adult HSCs and leukemia cells. Thus, many genes involved in normal stem cell differentiation and function are also involved in malignant transformation.

Two other signaling pathways, Wnt/β-catenin and Notch pathways, implicated in oncogenesis in both mice and humans, can also play central roles in the self-renewal of both normal and cancer stem cells. In C. elegans, Notch plays a role in germ cell self-renewal (Berry et al., 1997, Dev 124:925-36). In neural development, transient Notch activation initiates an irreversible switch from neurogenesis to gliogenesis by embryonic neural crest stem cells (Morrison et al., 2000, Cell 101:499-510). Notch activation of HSCs in culture using either of the Notch ligands Jagged-1 or Delta transiently increased primitive progenitor activity in vitro and in vivo, suggesting that Notch activation promotes either the maintenance of progenitor cell multipotentiality or HSC self-renewal (Shelly et al., 1999, J. Cell Biochem. 73:164-75; Varnum-Finney et al., Nat. Med. 6:1278-81). The role for Notch in human cancer is more complicated and less well understood. Various members of the Notch signaling pathway are expressed in cancers of epithelial origin and activation by Notch by chromosomal translocation is involved in some cases of leukemia (Ellisen et al., 1991, Cell 66:649-61; Zagouras et al., 1995, PNAS 92:6414; Liu et al., 1996, Genomics 31:58-64; Capobianco et al., 1997, Mol. Cell Biol. 17:6265-73; Leethanakul et al., 2000, Oncogene 19:3220-4). Microarray analysis has shown that members of the Notch pathway are frequently overexpressed by tumor cells (Liu et al., 1996, Genomics 31:58-64; Leethanakul et al., 2000, Oncogene 19:3220-4). A truncated Notch4 mRNA is expressed by some breast cancer cell lines (Imatani & Callahan, 2000, Oncogene 19:223-31). Overexpression of Notch1 leads to growth arrest of a small cell lung cancer cell line, while inhibition of Notch1 signals can induce leukemia cell lines to undergo apoptosis (Shelly et al., 1999, J. Cell Biol. 73:164-75; Artavanis-Tsakonas, 1999, Science 284:770-6; Jehn et al., 1999, J. Immunol. 162:635-8). It has also been demonstrated that activation of Notch-1 signaling maintains the neoplastic phenotype in Ras-transformed human cells (Weizen et al., 2002, Nat. Med. 8:979-86). Furthermore, in de novo cancers, cells with an activating Ras mutation also demonstrated increased expression of Notch-1 and Notch-4.

Wnt/β-catenin signaling also plays a pivotal role in the self-renewal of normal stem cells and malignant transformation (Cadigan et al., 1997, Genes & Dev. 11:3286-305; Austin et al., 1997, Blood 89:3624-35; Spink et al., 2000, EMBO 19:2270-9). The Wnt pathway has been suggested in MMTV-induced breast cancer in which deregulated expression of Wnt-1 due to proviral insertion resulted in mammary tumors (Tsukamoto et al., 1988, Cell 55:619-25; Nusse et al., 1991, Cell 64:231). Subsequently, it has been shown that Wnt proteins play a central role in pattern formation. Wnt-1 belongs to a large family of highly hydrophobic secreted proteins that function by binding to their cognate receptors, members of the Frizzled and low-density lipoprotein receptor-related protein families, resulting in activation of β-catenin (Cadigan & Nusse, 1997, Dev 11:3286-305; Leethanakul et al., 2000, Oncogene 19:3220-4; Reya et al., 2000, Immunity 13:15-24; Wu et al., 2000, Dev. 127:2773-84; Taiple & Beachy, 2001, Nature 411:349-54). Wnt proteins are expressed in the bone marrow, and activation of, Wnt/β-catenin signaling by Wnt proteins in vitro or by expression of a constitutively active β-catenin expands the pool of early progenitor cells and enriched normal HSCs in tissue culture and in vivo (Austin et al., 1997, Blood 89:3624-35; van den Berg et al., 1998, Blood 92:3189-202; Reya et al., 2001, Nature 414:105-11). Inhibition of Wnt/β-catenin by ectopic expression of Axin, an inhibitor of β-catenin signaling, leads to inhibition of stem cell proliferation both in vitro and in vivo. Other studies suggest that the Wnt/fβ-catenin pathway mediates stem or progenitor cell self-renewal in other tissues as well (Gat et al., 1998, Cell 95:605-14; Korinek et al., 1998, Nat. Genet. 19:379-83; Zhu & Watt, 1999, Dev. 126:2285-98; Chan et al., 1999, Nat. Genet. 21:410-3). Similar to their normal HSC counterparts, constitutive expression of an activated β-catenin increased the ability of epidermal stem cells to self renew and decreased their ability to differentiate. Mice that fail to express TCF-4, one of the transcription factors that is activated when bound to β-catenin, lack undifferentiated crypt epithelial progenitor cells, further suggesting that Wnt signaling is involved in the self renewal of epithelial stem cells (Korinek et al., 1998, Nat. Genet. 19:379-83; Taipale & Beachy, 2001, Nature 411:349-54). In humans, the Wnt/β-catenin pathway plays a role in tumor formation by human breast cancer stem cells isolated from some patients. Interestingly, members of the Wnt/β-catenin pathway are heterogeneously expressed by different populations of cancer cells and expression of particular members of the pathway can correlate with the capacity to form tumors.

The implication of roles for genes such as Notch, Wnt, c-myc and Shh in the regulation of self-renewal of HSCs and perhaps of stem cells from multiple tissues suggests that there can be common self-renewal pathways in many types of normal somatic stem cells and cancer stem cells. It is important to identify the molecular mechanisms by which these pathways work and to determine whether the pathways interact to regulate the self-renewal of normal stem cells and cancer cells.

The embodiments of the present invention including the compositions and methods of generating the multi-stage stem cell carcinogenesis system can be used to provide diagnostic and/or prognostic information for many types of cancer including, but not limited to, acute lymphobalstic leukemia, adult acute lymphoblastic leukemia, childhood acute myeloid leukemia, adult acute myeloid leukemia, adrenocortical carcinoma, childhood adrenocortical carcinoma, AIDS-related cancers, AIDS-related lymphoma, anal cancer, appendix cancer, childhood cerebellar astrocytoma, childhood cerebral astrocytoma, basal cell carcinoma, extrahepatic bladder cancer, child hood bladder Cancer, osteosarcoma i.e. bone cancer, malignant fibrous histiocytoma, childhood brain stem glioma, brain tumor—cerebellar astrocytoma, brain Tumor—cerebral astrocytoma/malignant glioma—childhood; brain tumor—ependymoma, brain tumor—medulloblastoma, brain tumor—supratentorial primitive neuroectodermal tumors, brain tumor—visual pathway and hypothalamic glioma, brain tumor—other, breast cancer, bronchial adenoma carcinoids, Burkitt lymphoma, carcinoid tumor—childhood, carcinoid tumor—gastrointestinal, carcinoma of unknown primary, central nervous system lymphoma—primary, cervical cancer, childhood cancers, chronic lymphocytic leukemia; chronic myelogenous leukemia, chronic myeloproliferative disorders, colon cancer, colorectal cancer, cutaneous T-cell lymphoma; desmoplastic small round cell tumor, endometrial cancer, ependymoma, esophageal cancer, Ewing's sarcoma (in the Ewing family of tumors), extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor—extracranial, germ cell tumor—extragonadal, germ cell tumor—ovarian, gestational trophoblastic tumor, adult glioma, childhood brain stem glioma, childhood cerebral astrocytoma glioma, childhood visual pathway and hypothalamic glioma, gastric carcinoid; hairy cell leukemia, head and neck cancer, hepatocellular (liver) cancer, adult (Primary), hepatocellular (liver) cancer—childhood (primary), Hodgkin lymphoma—adult and childhood, Hodgkin lymphoma during pregnancy, hypopharyngeal cancer, hypothalamic and visual pathway glioma—childhood, intraocular melanoma, islet cell carcinoma (endocrine pancreas), Kaposi sarcoma, kidney (renal cell) cancer, childhood kidney cancer, laryngeal cancer, Leukemia, acute lymphoblastic, adult; Leukemia, acute lymphoblastic, childhood; Leukemia, acute myeloid, adult; Leukemia, acute myeloid, childhood; Leukemia, chronic lymphocytic; Leukemia, chronic myelogenous; Leukemia, hairy cell; lip and oral cavity cancer; liver cancer, adult (primary); liver cancer—childhood (primary); lung cancer, non-small cell; lung cancer—small cell; lymphoma, AIDS-related; Lymphoma, Burkitt; Lymphoma, cutaneous T-Cell; lymphoma, Hodgkin, adult; Lymphoma, hodgkin, childhood; Lymphoma, non-Hodgkin, adult; Lymphoma, Non-hodgkin, childhood; Lymphoma, non-Hodgkin during pregnancy; Lymphoma, Primary Central Nervous System; macroglobulinemia-Waldenstrφm, malignant fibrous histiocytoma of Bone/osteosarcoma; childhood medulloblastoma, melanoma, melanoma—intraocular (eye), Merkel cell carcinoma, adult malignant mesothelioma, childhood mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma (cancer of the bone-marrow), chronic myeloproliferative disorders, nasal cavity and paranasal sinus cancer, nasopharyngeal carcinoma, childhood nasopharyngeal cancer, neuroblastoma, non-Hodgkin lymphoma, adult; non-Hodgkin lymphoma, childhood; non-Hodgkin lymphoma during pregnancy, non-small cell lung cancer, oral cancer—childhood, oral cavity cancer—lip and oropharyngeal cancer; osteosarcoma/malignant fibrous histiocytoma of bone, childhood ovarian cancer, ovarian epithelial cancer, ovarian germ cell tumor, ovarian low malignant potential tumor, pancreatic cancer, childhood pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, pregnancy and Hodgkin lymphoma, pregnancy and Non-Hodgkin lymphoma, primary central nervous system lymphoma, prostate cancer, rectal cancer, renal cell (kidney) cancer, childhood renal Cell (kidney) cancer, renal pelvis and ureter-transitional cell cancer, retinoblastoma, childhood rhabdomyosarcoma, salivary gland cancer, childhood salivary gland cancer, sarcoma—Ewing family of tumors, Kaposi sarcoma, adult soft tissue sarcoma, childhood soft tissue sarcoma, uterine sarcoma, Sézary syndrome, skin cancer (nonmelanoma), childhood skin cancer, skin cancer (melanoma), Merkel cell skin carcinoma, small cell lung cancer, small intestine cancer, squamous cell carcinoma (nonmelanoma), metastatic squamous neck cancer with occult primary, stomach (gastric) cancer, childhood stomach (gastric) cancer, childhood supratentorial primitive neuroectodermal tumors, cutaneous T-cell lymphoma, testicular cancer, throat cancer, childhood thymoma, thymoma and thymic carcinoma, thyroid cancer, childhood thyroid cancer, transitional cell cancer of the renal pelvis and ureter, gestational trophoblastic tumor, unknown primary site carcinoma of adult, unknown primary site cancer of childhood, urethral cancer, endometrial uterine cancer, uterine sarcoma, vaginal cancer, childhood visual Pathway and hypothalamic glioma, vulvar cancer, Waldenstrom macroglobulinemia, Wilms tumor, women's Cancers.

### VIII. Genetic Background of Cell Lines

One aspect of the present invention is that all the stem cell clones derived at each different stage along the stem cell carcinogenesis continuum as well as the normal unaltered stem cell counterparts originate from the same genetic background. In humans, there exists genetic polymorphism, which refers to the occurrence in the same population of multiple discrete allelic states of which at least two have high frequency (conventionally of 1% or more). An individual's genetic background can affect tumorigenesis through a variety of mechanisms, such as the processing of mutagens by the body, the rate at which mutations can be repaired, the effectiveness of redundant cell cycle checkpoints within the cell, or the interaction of the surrounding tissue with the growing tumor. In this way, polymorphism i.e. different alleles of certain genes can determine whether or not the individual develops cancer. For example, neurofibromatosis type 1 (NF1) is one of the most common human genetic diseases affecting the nervous system and is characterized by clinical heterogeneity, whereby one family member may have a severe form of the disease while another may have a mild form.

Genetic background of different cancer cell lines also influences gene expression profile. For example, the ability of different populations of breast cancer cells to form tumors differs. Genetic background of different cancer cell lines influences the gene set involved in chromosome 8 mediated breast tumor suppression (Seitz S. et. al. vol. 45 issue 6, pp 6120717, 2006). Mitochondrial genetic background also plays a role in modifying an individual's risk to breast cancer (Cancer Res 2007; 67(10):4687-94). In addition, genetic background of the host is important for cervical cancer susceptibility. Genetic factors that could participate in the susceptibility to this tumor and disease outcome include but are not limited to polymorphic genes of the major histocompatibility complex (MHC), and particular polymorphism in the tumor suppressor genes such as p53 gene. In mice, the phenotype of a given single gene mutation is modulated by the genetic background of the inbred strain in which the mutation is maintained.

Having large source of homogeneous cancer stem cell populations and their normal/unaltered counterparts from the same genetic origin, as described in one embodiment of the invention, provides a high-throughput and robust system that allows for highly reproducible results. In some embodiments, the methods of the present invention enable the generation of panels of cells with a variety of genetic backgrounds. In some embodiments, the genetic origin is of a type of variation selected from the group consisting of ethnicity, genetic polymorphism, predisposition to a disease, and genetic mutation.

### IX. Molecules Involved in Stem Cell Carcinogenesis

One embodiment of the present invention includes identification of molecules that are involved in stem cell transition along the stem cell carcinogenesis continuum. Cancer stem cell markers find use in the diagnosis and characterization and alteration (e.g., therapeutic targeting) of various cancers. Known cancer stem cell markers include, but are not limited to, Bmi-1, eed, easyh1, easyh2, mf2, yy1, smarcA3, smarckA5, smarcD3, smarcE1, mllt3, frizzled 2, frizzled 6, frizzled 7, mf2, Frizzled 1, Frizzled2, Frizzled4, Frizzled10, Frizzled6, FZD1, FZD2, FZD3, FZD4, FZD6, FZD7, FZD8, FZD9, FZD10, WNT2, WNT2B, WNT3, WNT5A, WNT10B, WNT16, AXIN1, BCL9, MYC, (TCF4) SLC7A8, IL1RAP, TEM8, TMPRSS4, MUC16, GPRC5B, SLC6A14, SLC4A11, PPAP2C, CAV1, CAV2, PTPN3, EPHA1, SLC1A1, CX3CL1, ADORA2A, MPZL1, FLJ10052, C4.4A, EDG3, RARRES1, TMEPAI, PTS, CEACAM6, NID2, STEAP, ABCA3, CRIM1, IL1R1, OPN3, DAF, MUC1, MCP, CPD, NMA, ADAM9, GJA1, CD14, SLC19A2, ABCA1, PCDH7, ADCY9, SLC39A1, NPC1, ENPP1, N33, GPNMB, LY6E, CELSR1, LRP3, C20orf52, TMEPAI, FLVCR, PCDHA10, GPR54, TGFBR3, SEMA4B, PCDHB2, ABCG2, ALCAM/CD166, alpha-fetoprotein/AFP, BMP-4, E-cadherin, β-catenin, CD2, CD3, CD31/PECAM-1, CD38, CD44, CD45, CD74, CD90/thy1, CXCR4, decorin, EGFR/ErbB1, endoglin/CD105, EpCAM/TROP-1, FcγRI/CD64, FcγRIII/CD16, FcγRIIIA/CD16a, FcγRIIIB/CD16b, GLI1, GLI2, IL3Rα, integrinα2/CD49b, and integrinα6/CD49f.

The present invention further embodies molecules that are preferentially expressed on cancer stem cells as compared to normal untransformed stem cells. Known genes whose expression is altered in passaged tumorigenic stem cells versus HSC include, but are not limited to, FN1, FN1, RAI3, KRT19, FN1, FN1, ITGB5, S100A8, S100P, CA12, TACSTD2, AGR2, S100A2, DC12, DSP, DUSP4, FLJ20151, IGFBP3, S100A9, CXADR, CYR61, BIK, PTPRK, SERPINA3, zizimin1, CD24, SYN47, HRASLS3, LGALS3, FLJ11619, LCN2, RARRES1, GOLPH2, HRY, TFF1, EFEMP1, STHM, IFI27, SFN, MGC4309, ABCC3, DKFZp564A176, CD24, MYO6, KRT7, MUC1, IER3, CTSL2, S100A11, MET, PRO1489, C8orf4, PPL, CD24, GPRC5B, S100A8, COBL, CDS1, TACSTD1, TACC2, KRT18, IL1R2, SOX9, SPUVE, CAV2, TSSC3, C3, CYP1B1, ITGB5, CD9, KRT6A, MAPK13, ARHGAP8, CDKN2A, S100A10, SFN, RDHL, SOX9, CEACAM6, FLJ20273, MGP, CAV1, F3, TGFBI, LGALS1, MYO010, S100A14, INHBA, TM4SF1, CXCL1, TUBB, PPIC, FLJ10052, IL1RN, DPP7, FXYD3, GALNT3, KRT6A, ANXA2, ANXA2, FER1L3, ANXA9, TPD52L1, HRY, PTPN3, EFNA1, C8FW, CDH1, EPS8, CLDN4, PTPRF, CCND1, CALU, GALNAC4S-6ST, DKFZp56411922, ASS, CAP2, FARP1, CRIP1, LOC51760, HOXA1, MIG2, ANXA2P2, TGM2, MUC16, PAPSS2, SNK, RAI14, CAV1, COL4A5, C4.4A, PTGIS, KIAA1078, SLPI, SAR1, RARRES1, DUSP4, ANXA2, FLJ10901, CD24, KRT6B, EPN3, ADAM9, EPHA2, TFAP2C, BMPR1A, PARVA, SERPINB5, ENAH, MARCKS, FAT, BF, TACC2, FLJ20171, NCKAP1, TONDU, PIGPC1, PARG1, EMS1, CTSL, LIF, EPB41L1, ISG20, ITPR3, LOC90957, CXCL5, PACE4, PHLDA1, HN1, CXCL6, VIL2, C1orf34, GNG12, ALDH1A3, TJP1, TM4SF6, ROR1, FLJ20151, LGMN, DUSP5, IRS1, GFPT1, CD24, ADM, GATA6, LAMC1, NRCAM, CRABP2, ARHE, MCP, YAP1, ADFP, CARD10, COL4A2, EDG2, PTGES, OSBPL10, IGFBP3, KCNK1, RAB20, RIL, NFIB, EFEMP1, CTSH, PDXK, SGK, DEFB1, KRT17, RAB25, HUMPPA, C12orf5, DLG5, KIAA0869, SLC1A1, PPP1R14B, KDELR3, RAB31, DDR1, TSTA3, CDH3, TFPI2, PPAP2C, SLC12A8, TM4SF1, FLJ22662, DDR1, S100A6, DD96, KIAA1078, VEGF, ARHGAP8, ELF3, RAB31, RIG, MAL, COL4A1, HBP17, LOC113146, ERBB3, RHCG, NR2F6, EMS1, MUC4, PLAB, STEAP, S100A7, NET1, FLJ11856, MGC5395, GPR48, DLAT, RIN2, NFIB, CEACAM6, CORO2A, TIMM17A, CLMN, FLJ13593, FARP1, E21G4, IL1RL1, DSTN, CYB5R2, TIMP2, KRT8, GFPT2, POLR2J, SLC6A14, ANXA3, LAMB1, FLJ21918, MGC10796, EPB41L4B, GOS2, SDC4, CCL20, TLE1, LAMC2, NMU, SPAG4, TRIM2, RAB31, EGFR, ZNF339, MGC35048, PLAT, PITX1, ZFP36L1, GMFB, PHLDA1, BNC, SLC11A2, LAMB3, TFPI2, FLJ22408, SAT, LAMP1, POR, TGFA, MYO6, KCNMA1, TPM2, TUFT1, GPR87, BZW1, KDELR3, ANKRD3, EGFR-RS, AKR1B10, RBP1, CDKN2A, CLDN1, AKAP12, SLC7A5, SEMA3C, ERBB2, GPR64, PLXNB1, COX5B, MGC11242, FACL3, PPARD, PPAP2A, EMP2, CASK, MT1H, TMPRSS4, PDEF, KDELR2, FLJ21610, TMEM8, GSTT1, KREMEN2, ECT2, PFN2, MT1X, MT2A, HAIK1, CNN3, PTK2, IL1A, S100A13, NDRG1, MID1, TNFRSF11B, SOCS5, MATN2, ME1, SEMA3F, ARHD, PP35, ZNF144, MLPH, PDZK1, SCD, CRYAB, HSPC163, RRAD, IGSF3, PCBD, ITSN1, IL13RA1, UGCG, EDG2, ANXA8, SSSCA1, LAMAS, KIAA0436, KIAA0599, ENDOG, SLC6A8, CALD1, FLJ11183, MGC3101, UMPK, EFA6R, NQO1, PTK9, MT1L, ELF3, CST6, ST5, NETO2, KIAA0802, MYO1B, NOTCH3, PTK6, KIAA1416, MYO1C, SUCLG2, KRT17, RHBDL2, AMOTL2, COL7A1, IL20RA, CD14, CEBPD, SMARCA1, ESDN, TNFRSF6, FLJ20591, PEG10, FOXA1, KIAA1026, FLJ21870, PBEF, TOB1, AQP3, LISCH7, TGIF, MYO1B, MPZL1, DDR1, CP, IQGAP1, P4HA2, BMPR1A, NEBL, PLEK2, EPHB4, AK3, BHLHB3, IL6, TAZ, PLS3, OSR2, SH3YL1, NQO1, PPAP2A, UP, SBBI31, KDELR2, KIAA0790, FLJ10292, SLC2A1, AQP6, P2RY2, MTAP, FLJ10718, DAF, MOB, MKLN1, TM4SF6, SQSTM1, OCRL, C21orf97, NMB, FLJ23186, SDC1, RIS1, PTPRF, KLK10, SCEL, MGST3, CSTB, HOMER-3, PON2, CASK, SSH-3, DPP4, HSPB1, MGC2376, LOC92689, RARRES1, LTBP2, BNIP3, HMCS, TGM2, TNC, ITCH, MRPS12, CTSB, SUCLG2, PPIC, SLC31A1, MGC14480, KIAA0440, EGFR, AK3, SRD5A1, FBP1, FLJ13984, UBE2H, H2BFL, MGC3103, NPD009, FCGBP, CDK5, ANG, TEAD3, DPP4, PRRG1, NQO1, KIAA0429, SUCLG2, IF2, ERO1L, CLDN3, SERPINE1, SFN, FHL2, HS3ST1, PDE8A, CLDN8, BAP29, RRAS2, RPL5, PIG11, PPFIBP2, DNAJB2, RRAS2, NID2, TOPK, MRPL19, NT5E, FN1, KIAA0103, CED-6, MAP4K4, PRSS8, COL13A1, GIP2, ROR1, UGCG, BCAR3, ISG20, CYP24, LIM, LOC57228, SERPINE1, SLC7A8, TJP3, ESR1, NPAS2, CKAP4, CLDN7, UCHL3, KIAA0143, RBSK, FJX1, NOL3, SLC39A4, FLJ12910, BNIP3, PLP2, FLJ22531, FLJ22028, JAM1, LMNA, KIAA0644, CUGBP1, VNN3, LAMC1, CX3CL1, THBS1, NUP50, SLC31A2, NNMT, THBS1, AMMECR1, KMO, MAPK13, KIAA1695, RCP, GTF21RD1, ARPC1A, MMP7, DKFZP434E2135, IF2, GLDC, PRSS11, TJP1, ATF3, PAX8, IL13RA1, ATP6V1C1, TST, SHANK2, ANK1, CRIP2, ChGn, GAS2L1, EPHB3, N33, CD59, GEM, EIF5, CENTG2, OAZ3, ASPH, SRPK2, B3GNT3, EDNRA, HSPC159, BACE2, ATP6V1C1, DP1, EHD1, DNAJB1, YKT6, KLF8, DDEF2, SRD5A1, RALA, CYP1B1, GPNMB, DKFZP564A022, FGFR3, ACP1, FLJ20366, TLR5, SCD, KIAA0882, KIAA1028, SC4MOL, MPZL1, RALGPS1A, SAR1, PTCH, SDR1, PDE4A, CELSR1, F12, FGF2, GCNT3, SNCAIP, DDR1, PBEF, MMP14, EGLN1, ELOVL1, ADCY9, FST, KIAA0716, HSPA1A, CNGA1, HNMT, KIAA0984, SIRPB2, HRH1, ITGA3, FASTK, LDLR, RGS20, MRPS17, ELMO3, AP1M2, TEGT, SH3GLB1, SMARCA1, UNC84A, GJB3, CAST, DKFZP564F0522, SLC19A2, HK2, ID1, ARNTL2, EVI5, KLK11, KIAA0703, NPAS2, MEIS2, CRIM1, GCLM, PARD3, EML1, RAD23B, AP1M2, S100A11P, YWHAZ, PON2, MTCH2, FLJ23153, TUBB-5, CDH6, SCD, KRT5, RNASEH1, LHX1, UBE2D1, TMEFF1, MGC4171, PGM3, KLC2, TNF, HSKM-B, IDH3A, KIAA0874, FLJ11773, PSMD5, HGD, PPP1R13B, TNFRSF12A, FLJ13841, MBLL39, SH3BP5, FLJ22418, CETN2, CAST, 1F2, LLGL2, SPATA2, SYNGR2, SLC16A1, FBXO26, C1orf27, ITGB5, LOC113251, KIAA1029, FLJ20623, SELENBP1, PCDH1, DAG1, TMSB10, SUDD, STK17A, LAD1, SQSTM1, THBS1, ARNT2, CGI-115, TRIP13, DSTN, CTNND1, SOX1, SOX13, SFTPA2, SLC2A10, CGI-141, MT1G, COL4A6, CTNNAL1, RIL, IL1RAP, SNRPD3, MAOB, G1P3, PIK3R3, FLJ21511, NAV2, CLDN3, VEGF, KIAA1609, MEF2A, SCARA3, CPD, FER1L3, KMO, NY-REN-45, JAG2, OSBPL2, YIF1P, FLJ10055, PSMD12, GRIT, LOC113251, FBXL2, PRSS16, PTPRG, FOXE1, EML1, GUK1, RHO6, TPBG, HRB, H_GS165L15.1, FLJ12571, MGC29643, SBBI26, MARCKS, PSMB3, SLC11A2, FZD2, KIAA0220, TMEPAI, MTRR, HMGE, BCL6, STK39, CELSR2, KIAA0895, ACP1, E21G5, KDELR3, CYP-M, ANXA10, ANK3, CLIC4, KRTHB6, TSTA3, MLFI, TES, ASPH, PAPSS2, SLC20A2, RGS19IP1, NFIB, NPD009, HOXB7, FLJ10134, APOE, KIAA1219, KIAA0173, PODXL, IGFBP1, HSPCA, MAK, C11orf5, HIG2, CRIM1, FKBP2, HSPA1B, FLJ20624, CPD, ITCH, ENSA, UNC84A, KIAA0062, EPPB9, FLJ10851, STK6, PSCA, PTP4A1, DNAJC3, FLJ13782, CKTSF1B1, UAP1, KRT15, AXL, HMGCS1, GNPI, PRKCI, MGC5509, MAGED2, CD63, FLJ11856, ADAM10, KIAA0934, DXS9928E, SYNE-2, IFNGR1, SLC7A11, RIG, PP1057, LOXL2, SPOCK, PTPRF, PACSIN3, ATP11A, STK24, CAPN2, C4BPA, FLJ11149, TMP21, CYP2E1, COL4A 1, PTP4A 1, KIAA0937, PKP2, ARF4, KLF5, HSPA4, NPC1L1, ATP5J2, MSLN, TLE1, ARKS, SS18, SNARK, LOC56902, KIAA1630, JAG1, KIAA0843, CIS, MAP4K3, TAZ, PTHLH, RHEB2, NEDD5, HOXB7, MGC24447, EIF2AK3, UGTREL1, MIG2, ADK, GAL, FTH1, FTS, PEN-2, TNFRSF11B, CGI-148, MGC11061, LAMP1, MGC39851, CPD, MGC11061, NCOA3, CDC42BPB, C11orf24, MAP3K8, MGC3038, TRA, IRS3L, CLTB, SC65, KIAA0471, PTS, POLR2K, CED-6, BLZF1, TRIM36, SPR, AP1S1, EVA1, LIMK1, TIMP1, KIAA0923, NDUFS8, EMP1, BFSP1, JAG1, GOCAP1, BID, RIL, CGI-90, CLTB, RIG-I, ANGPTL4, ATP11A, ITGAV, IL1RAP, SH2D1A, FLJ22693, INSIG1, FKBP10, FLJ20847, DUSP14, VDR, IFRD1, TOMM22, POLR2K, IGFBP4, HSD11B2, PTHR2, PREI3, FLJ10769, AFAP, ENC1, MFN1, CD24, H₂BFT, TRIM2, HIP2, JAG2, DAF, FLJ10099, CRK, YES1, DLG5, RARRES2, LIPG, APXL, FLJ20113, CYP51, CALM1, MKI67, PLS1, V1P32, WARS, ABCA1, RASAL1, CDC42EP4, MYOID, CRA, H₂BFB, KIAA0790, BOP1, TACSTD2, KPNA2, SGSH, RPP20, LAMP2, GRSF1, CBLC, ZNF165, SCAMPI, PLOD2, GSTM3, CLTB, C2orf6, MST1R, GSPT1, CLCA2, SGCE, CHST3, CDC42EP4, NPC1, TPM4, HEBP2, WBSCR21, HMGCR, ARL7, FLJ20623, DHFR, FLJ23548, IL8, DKFZP564F013, SECTM1, RAD23B, CFLAR, POU2F3, ITPK1, IGSF4, CBX3, RHOBTB3, PDP, HSPA4, WFDC2, TRIM16, ARHD, KIAA0632, TCN1, ITGB4, KIF5B, SGPL1, RAD1, EIF2S2, CYC1, IL1R1, HARC, KIAA0779, SLC25A13, PPARG, RAB17, PLEC1, DKFZP564A2416, C20orf97, DDX26, ALDH3A2, CGI-12, BAG3, EPB41L1, GS3955, FLJ20986, C14orf92, PP35, BTF, KRT7, FLJ20457, G10, EPS8R2, LOC160313, MGC2376, KIAA0429, GOLGA2, GOSR2, COX17, FLJ21313, FLJ10300, EIF5, SKD3, ADK, NPEPL1, SLC35A3, FLJ20186, YWHAZ, UBE2A, CYB561, NR2F2, ELK1, FLJ13397, LAMP2, SGSH, FDPS, FLJ10534, PIK3R3, SPINT1, FLJ11619, FLJ20989, ATIP1, SORD, PP, HCCS, SLC1A1, FLJ20739, SLC6A8, RBBP8, GRIK3, CALU, KIAA0644, SAA2, KIAA0934, USP18, TXNL2, FLJ10521, FBXO3, SSBP1, MGC3067, CGI-100, MRPL13, PIG7, KIF3B, KIAA1735, DAAM1, ADAM17, IL5RA, TPD52L1, PPP2R3A, RAB9A, PAWR, HIPK3, PPP3CB, EPHA1, GFPT1, KIAA0431, C7orf14, BNIP1, LMCD1, ATP6V1G1, COPB2, KIAA0265, RPL5, FLJ20234, OBP2B, MIR16, CTNND1, ATP6VOE, DHCR24, FRK, MGC5178, IQGAP1, HFE, DKFZP434J214, ACTL7A, APBB2, LANO, PMM2, HMGE, ARHGEF4, NPTX1, CTSB, RPA3, NET-7, ARHGAP6, FLJ20637, FLRT3, FLJ10407, RTP801, NR6A1, NR5A2, PTPN12, ZNF217, TEB4, CALD1, HSPC111, DPI, SNAI2, STS, ANXA4, BRIX, MGC16723, MCP, FLJ22055, C1orf28, ACTN1, TMEM4, FLJ20401, SE57-1, SH3GLB1, CDYL, OAZIN, PRO1855, H41, RAB22A, FLJ10326, PEX13, SH3BP5, MIF, SOAT1, MRS2L, CDC6, PEPP3, FLJ14675, TPD52, CTBP2, SPINK1, PPP2R1B, SELT, TNFAIP1, SORT1, ATP1B1, QSCN6, PDK1, SNX16, VIL2, PMM1, CIB1, FLJ22195, SLC27A5, PCNP, TNFRSF10B, CDR2, FLJ21657, MTX1, SLC38A1, BC-2, PEX3, CIAO1, PLXNB2, ROD1, RPL39L, TAF1B, ZF, C12orf22, DDX26, ME1, NPEPPS, DNAJB1, SLC39A1, ATIP1, MGC2742, BBOX1, FAM3C, FBXL11, EGR1, LIN7C, UBE2G1, MCP, TMPRSS3, MARCKS, LOC56902, GRAF, ALS2CR3, KIAA0680, FZD6, SPON1, HSPC111, CCNB1, P2RX5, B4GALT4, GOLGA2, p47, KOC1, RAB2, TM4SF9, MGAT4A, HS2ST1, CD44, FLJ20315, TCFL4, PCMT1, BHLHB2, VRP, RBSK, FLJ10829, HES2, EKI1, ZRF1, C2orf6, TUBGCP2, PFTK1, BZW1, CYR61, NOL3, PTGES, CGI-100, BM039, SCR1B, DDX3, SVIL, SMC6, NET-6, KIAA1023, ATOX1, IER5, IL1R2, STX6, PKP3, PITX1, ETV2, MCCC2, MRPL33, MGC2494, BPGM, C22orf2, ACTR2, BCL10, TRAM, B7, FLJ12439, DKFZp564A176, PHKA1, SLC33A1, TGOLN2, HRC, LGALS8, FLJ22940, OBP2A, STOML2, IFNGR1, POLR2J2, DKFZP586B0923, SLC2A4RG, NDUFA8, KIAA0964, FLJ11269, TMPRSS2, PLEKHA1, UGT2B28, ARL1, PFDN2, IGLJ3, FLJ23516, KIAA1609, WSB2, KIAA1598, YES1, KIAA0284, ATP6V1D, VMP1, C22orf5, HSPA6, MUC1, MAPK9, PARD3, APG12L, RAB5C, PAK6, LSM1, INSIG1, NDUFS6, ALDH3B2, TNFSF10, FLJ20275, CHML, UBE2V1, IGF2R, ITGB5, SEC61G, LOC55831, OPTN, ORMDL2, GABRP, DPP3, FLJ20967, POP3, GPC1, ANXA2P3, PRDX4, CHPPR, DKFZp434G2311, LGALS3BP, UEV3, KRAS2, TM4SF11, FLJ10116, CTBP2, CALU, USP3, P4HA1, SLC22A1L, FER, SLC1A7, PCDHA12, ENC1, FLJ14251, PPP2R3A, FLJ20069, DDXx, STK6, PLA2G5, ZYG, PPFIA 1, AFFX-HUMGAPDH/M33197_{—}5_at, AK1, GNA11, WWP1, HRY, SMURF1, FOP, DHCR7, GCSH, HDGF, NCBP1, ETEA, KIAA1096, GMPS, TGFBR3, HSF2BP, ZFP103, CD44, C20orf24, PSEN2, PEX7, TNFRSF21, ARHGEF7, CD2AP, ARF4, CHD1L, MGC8974, ZMPSTE24, PSMB5, ACR, GSK3B, NEDD4L, KPNA4, VIL2, CDC42EP2, UNC119, EPS8R1, KIAA0143, FLJ22709, LOC055862, YWHAE, BAZ1A, WIT-1, IL13RA1, ITGB8, OS4, LRP3, DRIL1, FASN, TXN, RASAL2, NCOA3, JUP, AUH, NEK2, GEMIN6, PSMD11, RECQL, MAP7, SNX4, TPD52, KLK8, INPP5E, KW1C, ORC5L, CDA, C20orf35, FLJ13189, B4GALT4, CDK5R1, C1orf16, ATP6V1D, KIF5B, CTNND2, CGGBP1, SQLE, PTP4A1, CSNK2A1, LIFR, PLSCR1, SRI, CDC20, PSMB7, C20orf18, NAT1, KLK5, KPNA1, PELI1, TRIM29, YWHAZ, KLF4, FLJ21916, LTF, DAPK2, DHCR7, RNMT, RXRA, SPAG1, DDX21, CKTSF1B 1, OXTR, KIAA1096, COL16A 1, CELSR2, KIAA0111, TPARL, MLCB, STS, DKFZP586C1619, TPSB2, MEIS3, APBB2, HSPC121, ASK, ABCB6, RBMS2, DKFZp762N1910, CCNE1, FLJ22347, TEAD4, PPM, NDUFS8, TMG4, BUB1, RRAS2, NOC4, SSH-3, TAX1BP1, EPN2, ISGF3G, MRPL17, AHNAK, TBL1X, EKI1, B4GALT1, SPHK1, PPIF, TXNDC4, DSC2, KIAA1096, SSR1, ATP9A, OSBPL1A, COX8, EIF2S1, SIP1, ACPP, FLJ20085, SMARCA4, SSTR1, UNG2, C1GALT1, PRKCL2, CAB YR, FLJ10232, SLC4A7, ARHGEF5, GLUD1, MED8, MAP2K1, PPM1B, NET1, PPP2R3A, RHEB2, PME-1, FLJ20591, FLJ22595, SPS, CPSF5, MGC5466, SLC35A2, PLOD2, DKFZP434B103, APPBP2, TFIP11, FLJ10252, MRPS16, KCNK1, GOLGA5, PAIP1, CHPPR, PA200, APP, FLJ23338, FLJ13852, RHEB2, PK428, BAIAP2, LAMC2, C7orf10, LANCL2, ITGB1, HCCS, TPM1, FACL3, MRPS15, EPPB9, ITGB1, FLJ10199, CSPG6, COPS7A, KRTHA6, SGPL1, EML4, AHCYL1, TPD52, SHC1, EPLIN, TUBB1, GAS2L1, MPZL1, IDH3A, CYP4B1, CGI-96, TM9SF2, FER1L4, C10orf3, FLJ23537, LGALS8, P2RY6, ALDOA, PEX7, EBNA1BP2, DKFZP566C134, NPEPPS, PDE4DIP, GSG1, FLJ20485, MTIF2, PCTAIRE2BP, FLJ23510, LAMP1, KIAA0020, GMFB, ACTR2, HLCS, P4HB, CYCS, PSMD8, TIMM17A, MFTC, TXNL2, PNAS-4, CGI-60, PMP22, TONDU, GGPS1, FLJ20604, TAT, FLJ10803, CLN5, NRP2, RPN1, KIAA1718, CALM1, NOV, MAOA, TPS1, FLJ20555, KIAA0649, TSLL2, OSBPL11, TPM2, MRPL40, TCF-3, H₂BFT, SLC4A7, SURF2, LZ16, KIAA0471, DPM1, DNAJA2, COGS, DKFZP434G2226, DC50, TCEB1, ACLY, DUSP3, ROD1, NCOA3, NFATC4, GAN, UNC84A, UCHL5, FLJ11850, RPP38, MYCBP, PDEF, DKFZP586N0721, KLK6, TPI1, PSMC2, SLC16A1, TEAD1, VEGF, NDUFS1, BS69, MAGEA3, TLE2, HSPCOS1, FN1, BAZ1A, FLJ22584, SEC23B, NMES1, MAL2, PIGPC1, LOC55971, FLJ20171, ShrmL, LOC91523, FLJ22474, H19, RHPN2, MIG-6, NGEF, KIAA1165, YAP1, MGC4309, SYNE-1, CDKN2B, ENAH, CTL2, ALS2CR9, TMEPAI, IMUP, DKFZP564J0863, UGCG, MGC12335, ITGB6, CYP4X1, GLIS2, FLJ20273, FLJ31842, LOC55971, TMEPAI, SYT13, SPUVE, KIAA1244, HSJ001348, MGC29643, BOK, TEM8, FLJ30532, LBP-32, DKFZP761L0424, FLJ23153, EDG3, IL20RA, MYOSB, GJB2, MYEOV, PTK2, KIAA2028, SBBI31, FLJ10052, AGR2, FGG, FAD104, LOC120224, CLDN1, LOC51760, IRX3, C20orf100, CLDN12, MGC4734, ERO1L, FLJ40432, MGC33630, NTN4, KIAA1522, SLC4A11, ESDN, DKFZp434C0328, PTGFRN, EHF, MFI2, PRO1489, TCEA3, GNG12, TMPRSS3, TEAD2, GJB6, ALS2CR9, DDEF1, CFL2, LOC116238, KIAA1671, SDCCAG43, MGC35048, TOB1, LRG, DKFZp761PO423, C20orf129, SMOC2, FZD4, RDHL, WNT7B, MGC14839, DJ667H12.2, TEAD1, RDHL, FLJ14957, ZIC2, HSPC163, DLG5, FLJ14735, FLJ20048, WW45, FLJ90440, LOC92689, DAG1, LOC55971, B4GALT1, HAS3, PIGR, SNX9, AK2, PRO2605, UGCGL2, CDH24, GFRA3, FLJ13593, CP, CRBPIV, FHOD2, MGC26963, LOC129642, UACA, YAP1, FLJ23420, IL28RA, PSA, DKFZp434D0215, PPP1R14c, PTGFRN, E21G5, C14orf31, FLJ10052, BCAR1, MGC22805, DKFZp434G171, MGC11034, KIAA1870, FLJ22415, FLJ34633, GPR54, CHDH, FST, KIAA1708, UBE2H, DDEF1, WASL, FLJ14408, CXCL16, PARVA, DKFZP434H0820, CASPR3, RAB10, PDP, ANLN, FLJ25157, NETO2, OLD35, UBQLN1, LOC58489, FLJ23867, E21G5, ATP11A, CD44, DNAH5, LOC128153, PHLDA1, IPP, DUSP16, COL12A1, MGST1, PLEKHA1, KIAA2025, LTB4DH, FLJ20739, FLJ22174, MGC24180, DKFZp761NO624, IRAK2; ALS2CR9, MGC39329, AKAP2, C14orf50, MGST1, UGCGL1, KLK7, FLJ31937, DIRC2, FLJ10035, MGC11034, SOX7, PARVA, LOC139231, GPCR1, SDCCAG28, GPR92, LOC147184, LOC113026, MGC14798, LOC147700, DKFZP434A1315, FLJ10702, LTB4DH, PYPAF3, RBMS1, SLC30A1, MTA3, ARL8, KIAA1688, RASAL2, PDK1, XPR1, SULF2, STEAP2, H41, METL, FBXO32, TLE1, DDEF1, GPT2, MRPL30, FLJ14117, DKFZp434E2321, MGC26963, SAT, ORF1-FL49, GRP58, MGC33662, NT5E, FLJ31052, RNAC, CGI-85, CTL2, STC1, SCD, DKFZP434K0427, SCARA3, MGC14128, BCCIP, MGC3195, TGFBR3, PXMP4, KIAA1500, Spir-1, ARHGEF12, DKFZP434A0225, LOC55829, C20orf24, HSPC242, CAMK2D, FAD104, ZD52F10, HS6ST2, HLCS, FLRT3, SDCCAG28, KLF15, C20orf139, FLJ39155, MGC1314, C20orf24, FLJ14511, CGI-20, EDG8, MGC10765, C7orf3, MGC14801, FLJ10697, ATP1B1, EHF, JUB, FLJ11200, MacGAP, H4FH, MGC11102, RORC, COL12A1, PRO1853, MGC13096, SPTB, FLJ32115, DKFZP566F084, SEMA4B, DKFZP434A0225, BTC, PCDHB14, CGI-09, EMS1, PCDHB16, KIAA1384, SCEL, GRP58, KIAA1357, CAC-1, SURF4, FLJ11011, LMLN, ARL61P2, OCLN, C17orf28, INPP4B, C14orf31, FLJ22558, FLJ10116, KIAA1363, DAB21P, MGC35352, GK001, PDGFA, SNX8, MGC22805, LOC114990, ELP2, CXADR, LOC120224, ST6GalNAcI, MGC35403, MGC39350, KPNB2, DSCR1L2, FLJ20333, PPP1R1B, EIF2C2, PX19, BPNT1, AD-003, LACTB, FLJ36445, ULBP2, GUK1, KIAA1321, SPP2, CRB3, FLJ90586, NDUFB9, PDK4, FLJ30973, HSPC228, MacGAP, DEFB118, DKFZp761K2222, ASPH, MGC45474, UBQLN1, TRAF4, DKFZp761K2222, DJ667H12.2, AFFX-HUMGAPDH/M33197_{—}5_at, C12orf22, RHOBTB3, MGC33974, KPNB2, C9orf5, FLJ32421, FLJ25604, COQ4, FLJ20281, FLJ13391, TEAD2, ELL2, RPS3A, FLJ33516, ESPN, DKFZP434A0225, KIAA1684, TRA@, SEC61A1, DKFZP434K0427, PRIC285, KIAA1870, AMN, LOC151242, FLJ20686, FLJ10210, FLJ22415, MGC19764, CGI-97, CDW92, NAT5, KIAA1126, CLMN, RAB18, MRPS15, JAM1, TEAD2, ENAH, KIAA1228, ACTR3, PCDHA10, ATP5A1, GNPNAT1, CL25084, LOC51260, CNN3, TFDP1, FLJ31528, KIAA1434, FLJ10902, MGC14289, GGTL3, SYTL2, MGC21874, TIM50L, PHCA, PSCD3, KIAA1026, INADL, DNAJC5, AD037, FLJ11046, KIAA1804, KIAA1337, PPARD, KIF1B, MIR16, ROD1, SLC2A13, CFL2, GDF1, MRPL36, SLC26A9, LOC51290, CABYR, HSPC159, SPPL2A, ABCC3, BTBD6, SMURF2, STK35, CGI-85, ZAK, DKFZp434B1231, KCNK6, PCDHB2, Spir-1, KIAA0146, ZNF265, COPZ1, FLJ20421, C11orf15, DKFZp761D0614, KRT19, RAB23, MGC16491, FLJ40432, MGC10981, C20orf45, CTEN, MGC30022, NUCKS, MGC13251, MRPL27, FLJ90586, MGC16028, FLJ90165, SHMT1, FLJ14525, BACE2, ABLIM2, FLJ20719, SCGB3A1, MGC2477, FLJ20038, MGC29643, FLJ30829, C20orf155, PGK1, FLJ37440, RBM8A, FBXO22, KIAA1219, KIAA1200, KIF3B, MGC19825, AK5, C22orf20, FLJ10378, INADL, HSPCA, E1F5A2, RAB18, BCL2L13, MBC3205, UBE2H, FLJ20354, SLC5A7, FLJ30532, C14orf47, TMPIT, EHD4, FLJ13089, MGC17299, IDS, CED-6, MGC27277, LOC137392, FXYD6, MGC22825, CPM, SNX9, MGC19764, TLR7, FENS-1, SDCBP2, NUDT5, MGC11102, SEC24A, CGI-141, NKD2, EFG1, ANAPC11, MYO5B, MGC14833, LOC85865, EPB41L4B, FLJ21415, KCNC4, GSBS, TEAD2, LOC115548, MAGI-3, C9orf5, CLONE24922, MRPS15, RGNEF, CORTBP2, FLJ20354, HSPC121, NOC4, KIAA1673, MGC14595, MGC2560, MGC2408, MRPL14, APOA1BP, FLJ14681, MGC13102, KIAA1437, KIAA1126, MGC13034, CSEN, SH120, VIP, PRO2000, SLC31A1, AD-003, CALM2, HT002, RAP2A, EML4, WDR5, MPP5, LOC90990, MGC2560, FLJ14431, ARHGEF5, HCC8, TCEB2, FLJ13187, FLJ90575, FLJ10525, FLJ23393, HOXB9, LOC84661, dJ55C23.6, HFE, MGC13040, WDR20, MRPL4, FLJ25604, DKFZP566C134, LOC55871, CGI-09, MRPS23, MRPL47, MGC13045, ERK8, KIAA1500, HPS3, CRYPTIC, SBBI31, MGC14353, CGI-20, FHOD2, PPP1R14A, REPS1, MAPKAP1, V-1, FBXO25, BNIP-S, MGC13114, EKN1, GPR24, RCP, FLJ12806, MGC2747, OBP2A, HM13, C21orf97, FLJ14909, C9orf10, STYX, THOC3, RDGBB, PFKFB4, FLJ21924, KIAA1295, ZDHHC9, STXBP5, RPE, UBE2H, PCDHB18, FLJ20303, NPD007, N4WBP5, FLJ20333, FLJ12747, SURF4, C20orf45, FLJ112787, LOC90507, FLJ10839, EPB41L4B, FLJ37953, BAP29, MRPL50, MGC10999, C9orf5, TBDN100, STK35, FRABIN, JUB, PRO2714, MLLT4, MGC40214, CPNE4, FLJ22233, MIZIP, MGC14859, MRPS24, HPS3, FLJ23841, FLJ23577, HSPCA, MRPS10, FLJ14251, SSR3, MGC13186, KIAA1453, HN1, HOOKS, ATP1B3, MRPL50, MAP4K1, LOC90120, D1S155E, DKFZP56400463, FLJ23816, CFTR, MGC40555, MGC20781, FLJ20085, NOPE, FLJ14825, MSP, LMO7, C7orf2, MRPL32, FLJ10074, MAK3P, KRT61RS, DKFZp547A023, SAMHD1, HSPCO43, FLJ10597, FACL6, LGR6, SORCS2, MGC4840, RAB35, MGC10911, and MLL3.

Genes whose expression is downregulated in passaged tumorigenic stem cells as compared to HSC include, but are not limited to, MEF2C, HSPCO53, HOXA9, PRG1, RetSDR2, GMFG, AIF1, A1F1, HLA-DPB1, PLCL2, ICAM2, HLA-DPA1, PTPRC, SPINK2, SPARC, CUGBP2, PTGER4, CECR1, CDW52, CCND2, LYZ, SELL, CD69, HOXA9, ITM2A, HLA-DQB1, ITM2B, LYL1, KIAA0125, LMO2, ARHGEF6, KIAA0084, MPL, RGS2, LAGY, QKI, EVI2B, ZNFN1A1, DOCK2, HLA-DRB3, NAP1L3, HLA-DPA1, KIT, HF1, HLF, LST1, ANGPT1, CD53, LST1, FLJ14054, SELPLG, LST1, BM046, TUBA3, HLA-DQA1, BCE-1, CDW52, FLJ10178, PRKACB, PRKCB1, IQGAP2, CHES1, GUCY1B3, PSCDBP, HLA-DRA, LAPTM5, PRG1, MEF2C, SLC2A5, LST1, FHL1, MAP4K1, TNFSF4, PLAC8, HLA-DQB1, IGFBP7, PCDH9, MAP4K1, EVI2A, SATB1, MLC1, SSBP2, FLI1, CLIC2, CLECSF2, LY75, NDN, HLA-DRB1, FLJ21276, DLK1, GLUL, NUDT11, BEX1, SH3BGRL, PRKCB1, MPHOSPH9, LST1, HLA-DQB1, FLJ22690, UQCRH, FLJ22746, HLA-DRB3, SLC2A3, NPIP, BCL11A, MPO, RUNX3, ERG, SV2, HLF, MMRN, CYFIP2, HLA-DRB4, PECAM1, CORO1A, MOX2, SEPP1, BAALC, 6-Sep, ITM2B, LCP2, PELI2, C17, IGHM, LRMP, PPP1R16B, HLA-DRB5, HBB, DJ971N18.2, LOC51186, SCGF, ERG, LAPTM5, P311, SAMSN1, ITGA4, DJ434014.3, IGFBP7, TFEC, HA-1, MAGED1, HSPCO22, FNBP1, TCF8, ELMO1, CUGBP2, NGFRAP1, PIP5KIB, DDO, MLLT3, ALCAM, NPR3, CMRF-35H, DPYD, PLAG1, BIN2, ITM2A, MYCN, GSPT2, LXN, ALEX1, PIK3CD, ADAM28, PLAGL1, FLT3, WBSCR5, C6orf37, GUCY1A3, CD74, KIAA0053, TRAITS, HLA-DQB1, MGC2306, ICAM3, PTGS2, H₃F₃B, TCF4, SNCA; FLJ10713, PROML1, TEK, APOBEC3G, PRO1635, HLA-E, JAM3, UBE1L, BCL11A, GNAI1, LHFP, LST1, CDH2, MYB, FLJ10462, ZFHX1B, CBFA2T3, TMSNB, HLA-DMA, PLCB1, SOCS2, CG018, PDE4B, MHC2TA, PADI5, USF2, CUGBP2, VIM, HLA-DRB6, TFPI, BIRC1, PTGS1, HFL2, SCDGF-B, LSP1, NRLN1, MPO, KIAA1939, PTGS1, MS4A3, HPIP, FLJ20220, HLA-DPA1, NCF4, MAPRE2, ZFP, BANK, TOX, CXCR4, IGHM, RUNX3, HCLS1, LOC81558, ARHGD1B, TRO, SCHIP1, CRHBP, KIAA1750, BCL2, FLJ20950, FLJ10097, DAB2, BASP1, JAM2, FLJ21616, HHEX, ITM2C, SPRY1, SERPING1, SLA, EBI2, ZNF42, DSIPI, FLJ10038, PECAM1, 6-Sep, CASP1, RB1, TACC3, 13CDNA73, 6-Sep, MAPRE2, FCER1A, BTK, LOH11CR2A, LRMP, PLAGL1, MICAL, TCF4, CLGN, H₁FX, WASPIP, LAIR1, ZNF175, INSR, FLJ20456, C11orf8, KIAA0443, AKAP7, TALI, HLA-DRA, HRB2, PLEK, RAGD, PLAGL1, ALDH1A1, B4GALT6, GLIPR1, GAB2, KIAA1157, PPM1F, WAS, SETBP1, MUF1, C6orf32, MYOZ3, TUCAN, RNU2, KLHL3, TSC, PKIA, MLLT3, NEFH, DKFZp564B0769, PPM1F, SNTB1, PCDH9, CRYGD, MPP1, ABCB1, KIAA1110, ALEX3, ATP2A3, KIAA0308, MAGEH1, BIMLEC, CTSW, SORL1, FLJ20898, MCM5, CD244, PPP1R16B, MAGED1, ASC, GIPC2, RASSF2, LOC81691, SCGF, PTEN, 24432, STAT5A, 6-Sep, SLC24A1, UBE1L, CD83, TAHCCP1, GNA15, NR3C2, KIAA0053, INPP5D, CPA3, GYPC, SYK, PRKACB, RUNX1, RIN3, TRB@, NPIP, CABC1, HLA-B, PGDS, CD34, SPN, L0058504, MAGEL2, TBXAS1, MFNG, LOC91316, TRAP-1, RECK, TCEA2, FLJ20136, ARHGAP6, AMT, CAT, ADARB1, PTEN, LCP1, CCL3, SCN9A, RASGRP2, D=58612223, SS-56, SLA, C4S-2, PDGFC, LILRA2, RAGD, HNRPDL, ZNF288, ITGA2B, LOC81691, HBD, SELP, C6orf32, PDZ-GEF1, CPT1A, KLF2, ZNF198, TACC1, HBB, B1, CIAS1, HNRPA0, HLA-DQA1, KIAA0308, MYO1F, PRO1331, RAB33A, TNS, NAP1L2, CERK, MGC4170, ADA, RNASE3, NFE2, ANKRD6, AKR1C3, CDC42, HIS1, TRIM22, BIN1, ICAM4, IL12RB2, CSF2RB, EPB41L3, BRDG1, TNRC5, CIRBP, RPLP2, AMPD2, SFRS7, EDG6, BRCA1, MSN, HLA-DQB1, C5orf5, GSTM5, ITPR1, IL16, AIF1, NFATC1, LILRB2, FGF23, STAC, RPL22, PTEN, LRBA, PFAS, CGI-116, DKFZP586A0522, MGC13024, GALC, ABCG1, MGC45806, ELF1, SAP18, ALDH5A1, ELA2, GATM, CHC1L, KIAA0918, LOC51334, FOSB, PRO2198, TEC, SLC1A4, CAD, KIAA1028, VAV1, LOC57100, C11orf21, SLC1A4, TRPV2, EPB41L2, FBN1, CD48, GIT2, CSF3R, DNAJC6, BIN1, KIAA0582, ARL4, SH3BGRL, GLS, FXYD6, PF4, SCGF, NEK9, PKD2, MATK, BIN1, NSBP1, MSH5, PRKG2, NT5M, PML, CD37, SF3A2, PLSCR4, CSK, HA-1, NUDT1, SIAH1, MEIS1, IGLJ3, HLX1, SV2B, DKFZP58612223, KEO4, ENPP2, CTSF, IL1B, PSMB10, IL1B, ZFP36L2, SFPQ, FLJ11175, ATP2A3, STK10, FLJ22021, MYOM2, PTENP1, MGC861, HERC1, Jade-1, BTEB1, KIAA11102, NPTX2, UCHL1, LYN, COL5A1, ZNF215, MGC2217, SRISNF2L, LOH11CR2A, RERE, COL5A1, RAP1B, CLDN15, VWF, HHEX, SMARCA2, SMCY, UBCE71P4, LOC115207, KPNB1, ZNF22, STOM, C16orf5, ICAM2, KIAA1102, CENTB1, DKFZP434C171, ITGAM, TFPI, CASP1, CLN2, TAL1, AASS, SAH, FLJ11712, FXYD5, KIAA0303, FBXL5, SFRS5, FNBP1, FLJ11749, MAGE-E1, SNRK, SPN, CTSS, SIAT1, SCARF1, HSPC047, CD38, VAMPS, SF3B3, FLJ10374, FHL1, PTPRCAP, LRBA, DUSP6, PTPRC, KIAA0092, PLA2G4A, RBM5, FLJ21478, PLCB2, GOLGIN-67, RBM8A, OXCT, HEMI, DUSP6, CR11, RAB6IP1, IMPDH2, C21orf33, LOC93349, EMP3, NASP, MGC40204, PTGER2, COL5A1, SPARC, NISCH, SIGLEC5, CSTF2T, HGF, SNX10, DACH, NINJ2, MGC12760, KIAA1332, NPIP, KIAA0379, LYN, H2AFY, PACAP, PLCG2, PDE4D, LOC129080, FLJ11753, KIAA0447, BCL2A1, FUS2, PTPN7, WASF1, ZNF42, C18orf1, UROD, KIAA0303, NRGN, RNASE2, FLJ23056, FYN, DEFCAP, PTPN22, MAPKAPK3, ZFP36L2, AF1Q, NCF4, CDH7, DJ971N18.2, PA26, ANXA6, PHGDH, MCL1, LEPROTL1, HUMMHCW1A, TNFRSF14, STK17B, CGI-49, MGC14258, PSIP2, CR11, FLJ35827, CCRL2, PTPRN2, CES1, SCA1, FLJ21865, KIAA0798, BIA2, HLA-DQB1, UCP2, DPYSL2, FLJ11259, FLJ20312, K1AA0240, GTL3, C6orf48, AK2, TFR2, FLJ13949, MAX, CHKL, FLJ12668, ALDH2, NUCB2, HPIP, RNF8, C1orf21, AS3, ZNEU1, FLJ11323, FLJ23506, LOC115648, KCND1, STMN1, BTN3A3, MAP4K1, ALG12, ATP5G2, PET112L, TIAF1, KIAA1043, TRPC1, THY28, SYT11, HSU79274, PRPF8, CLC, PCNT2, H2AFY, DAPK1, CCL4, RPL28, IFRG28, CCND3, C14orf94, MGC3035, 6-Sep, GNB5, KIAA0916, EIF3S7, LENG4, FACL5, AP1S2, MCM5, DKFZp434NO62, AIP1, PRO51, CIRBP, REC8, SLK, C11orf2, dJ222E13.1, H2AV, NEK1, BN1P2, FLJ13197, ITGA4, FLJ21269, KIAA0708, IMPA1, FLJ12750, SLC18A2, EMR1, KIAA0239, RPS9, ARHH, MCJ, ALTE, KCNE1L, ABCB1, RPL22, KIAA0841, LOC58486, SNX26, ADAMTS1, USP4, STXBP1, ITGA2B, C5orf6, RBM10, FLJ21439, KHK, OS4, MAPK14, NIP30, KIAA0471, SLC16A7, RIN3, DDX28, HPIP, RNASE6, ADSL, ARHG, GNG7, RHOBTB1, CACNB2, DATF1, PDZ-GEF1, RPL13, TALDO1, DGKG, FLJ22794, PTPN6, SYT11, C5, FLJ22349, FGFR4, CGBP, PRO12, LARS, RPL3, JIK, MGC45806, MGC2488, MGC2752, TYMS, PECAM1, NSMAF, ABCC1, LEPR, MYB, LAIR1, LOC57209, EP400, ALCAM, ZNF187, FLJ13386, KPNB1, LTA4H, HGF, PP1628, NRIP1, GNAO1, IL3RA, CD79B, CENTB1, ZNF261, ST18, FGF9, CDK10, RAI17, STARD5, OXT, PML, KATNB1, ASMTL, NEDD4, ACTA2, MBNL, FLJ31821, PER1, MOAP1, DCK, DXS1283E, SNCA, AD7C-NTP, MYBPC2, STX8, ATPAF2, ACYP1, RAD51L1, CLIPR-59, FACL4, AASS, RAC2, MGC2306, SLC27A2, FLJ23018, RGS1, NAP1L1, ELAC2, LOC51185, SGKL, PCDH16, TRAF5, KIAA0682, DGKZ, FLJ10539, PIGN, FLJ10647, NCOA1, LBR, GFI1, MAN2A2, KRTAP2-4, HLA-C, FLJ35827, PCDHA10, HLA-A, APLP2, SFRS5, FLJ13262, WTAP, EFNA2, C12orf8, CCND2, PTPRC, MPPE1, HMGA2, CLK2, SWAP70, PRO1843, FLJ14280, FLJ23277, KIAA1172, PRCP, MADD, SMARCA2, WASF2, MGC5149, CDC42, PLEK, SMARCF1, RCD-8, ATP9B, 1HPK2, IGHG3, DHRS4, EEF2, QARS, KIAA0841, ADRA2A, RPL29, GCNT1, UBL3, GRB10, IMP-2, ABCA5, HSPC157, TNFRSF5, H2AV, JM4, TBXA2R, SLC1A4, RPS6KA5, IGLL1, MGC8721, PEPP2, USP7, PSMB8, ARHGDIG, HLA-A, RBM10, NAP1L1, KIAA1393, AVP, KIAA1018, RPL28, RES4-22, NAP1L1, ST13, KIAA0186, MBNL, HEXA, KIAA0555, FLJ20189, MN1, TSPYL, USF2, APLP2, ZNF135, HPS1, RPS21, MAP2K5, HSD17B8, PROSC, NAP1L1, DUT, KIAA0170, TPK1, NY-REN-34, RBIG1, IL16, AKR7A2, STK10, PRP17, WWP2, PTD015, CAPR1, ARHGAP8, FLJ20856, APPBP2, LRRN1, MDM1, HLA-DMB, CGI-30, COX11, DDX28, ACK1, TM7SF3, FLJ23554, SDCCAG8, FLJ20094, MMP28, MUTYH, CA1, AKR7A2, WDR6, DYRK1A, DPH2L1, RBPMS, FLJ20005, MAP2K5, C4ST, FLJ22059, FLJ20202, H₂BFQ, CAMLG, CHAF1A, ABLIM1, MAPK11, RAP140, DUT, ITSN2, EHHADH, DKFZP547E2110, H2AFJ, MGC4659, RPL13, KCNA3, BC008967, CASP1, NMI, NBEA, NUMA1, DEF6, PRAX-1, TBC1D5, KIAA0332, NEW1CP, KIAA0769, CENTB2, CKIP-1, EIF4A2, OAZ, ARH, KIAA0467, C19orf7, KCNAB2, TTLL1, FLJ10597, SF3A2, FLJ11222, PSTPIP2, BCL11A, SPHAR, GLIPR1, KIAA0555, MMP2, EIF4A1, STOM, ALOX12, FLJ11588, RBAF600, PROSC, CG005, VILL, FLJ12707, M6A, TCIRG1, HTR1F, RICH1, F13A1, CACNA2D3, RRP4, TAF7, ZNF134, HSU53209, LZTFL1, TKT, LILRA2, ZNF302, FLJ13114, ZNF177, PURA, DKFZp5471014, TXN2, TLR3, BHC80, MGC5139, PTPNS1, ZNF145, THTPA, BTBD3, MDS010, KIAA0924, ZNF292, ITGB2, TJP4, GPRK6, CYLN2, ENPP4, ALB, RPS20, FOXO1A, ADH5, CTSS, FLJ23221, C11orf8, TNFSF13, TOLLIP, KIAA1449, HINT1, GLTSCR2, KIAA1052, FLJ10260, RAB3GAP, HINT1, TAPBP, CHD5, LOC57406, TP53TG1, SRP46, MS4A4A, NUP62, PIM1, ZNF42, COG4, ADPRTL1, ZNF289, CATSPER2, TXNIP, PDE4DIP, HSA250839, FUT4, HSPA1L, GALT, MGC4278, APEX 1, FN5, STRIN, USP11, SPP1, NPFF, CEP1, GAPCENA, HLA-E, SCAND2, CG005, VRP, BRAP, GPR56, MLH1, GPR105, OGT, C1R, BTN3A1, FLJ14107, PACS1, MGC26766, FLJ22378, APOBEC3C, CG005, CA11, QDPR, DUT, ALDH6A1, FLJ10450, BST1, NGLY1, FLJ12057, FECH, ZNF137, SERPINB1, EZH1, CASP1, MGC3265, CXorf9, TRG@, DKFZp564B0769, KIAA0616, D1S155E, MN7, C18orf1, NSBP1, NXF1, FHL1, TOP3A, TARBP1, KIAA0766, RRAS, SEMA4D, CEBPA, TIP120A, IL15, HADHSC, HIRIP3, CTBP1, DVL2, RBM12, RAD54L, NYD-SP15, PHC1, KIAA1042, IGL@, NPR3, HRMT1L1, FLJ20551, MYST1, LOC51231, TCF12, KIAA0543, MKPX, LOC51157, SYNGR1, AKR1A1, SCOP, LRRN1, FY, AMYIA, PHEMX, KIAA0930, MAP3K3, FLJ10631, ZNF85, APOL3, MAPK12, TRG@, POLD1, LDOC1, POLA, TPST2, WASF3, RPL11, MKL1, FLJ22242, PTPRM, AMHR2, FLJ20288, TERF2, DOK4, KCNAB1, DISC1, FLJ22494, LOC91316, VIP, POLR2A, RGS19, C12orf6, RPS9, LIG1, NASP, ARHGEF9, MANBA, SARM, SRPR, CDH9, MRPL16, FLJ20509, SNRPN, HLA-E, NTS, ZNF232, FLJ12903, PHKA2, MSH5, PURA, ATP9B, TRIM28, FLJ12768, ME2, IDS, MPHOSPH9, DIA1, ADAMS, HADHSC, STX12, COX15, RPA2, SHANK 1, GGA1, LANCL1, UBE3A, SOX11, LAT, BCL7A, DKFZp434K1210, BRAP, SMARCC2, DKFZP434H132, NHP2L1, FLJ11294, FLJ12270, KIAA1649, SRP46, PSMB9, GGA1, MGC4368, TOP2B, PTK2B, FLJ13912, EZH1, THRA, BAX, NAG, MERTK, HADHA, SRRM2, HNRPH3, GNG7, HSPC018, FLJ22573, HPCAL4, MBC2, MAPK4, FLJ10716, ITGAL, NFRKB, MRP63, DKFZP434L187, GABARAP, CHD4, DKFZP564D172, FGL2, LOC57019, KIAA0478, NTSR1, LPIN1, USP4, KIAA0391, ASGR1, KIAA0174, TBXA2R, TRAP95, FLJ22649, NEK3, ZNF271, SIL1, 76P, CYLD, CD164, TINF2, ZNF220, DAB2, HRIHFB2206, SF3A3, TRO, FLJ13373, UBE4B, GC20, ADAM28, PHKB, BCAS3, MGC14258, RAD52, HLA-F, KIAA0721, MRC1, CHD1L, LMOD1, FLJ10315, CHRNA7, NAP1L1, PIB5PA, GADD45A, RPL35A, LPIN1, TFPI, FLJ14213, KIAA0746, KIAA0981, C22orf4, PP1044, ABCF2, FLJ10379, RASSF1, FLJ23392, RPS8, DAB2, FLJ14011, CDC2L2, GAD1, MGC17330, FLJ23342, HEI10, NPDC1, KIAA0710, BIRC1, KIAA0349, SF3B3, MST4, IRAK3, CD81, LOC57406, FLJ12610, SF1, SLC27A2, KIAA0804, KIAA1055, GTF2F1, SEPX1, SCAMP2, PPP3CB, U5-200KD, HMGN2, F2, PCBP3, FLJ20721, ING4, HADHSC, KIAA0286, TREX1, ATP11B, RUFY2, SUPT3H, SFRS11, PIAS1, HBOA, HAS1, HYMAI, NUP210, TGT, FLJ11896, CIDEB, TRHDE, FLJ90524, TOX, KIAA0261, GSTM2, GAS7, MBD1, KIAA1305, PPP2R2B, CDT1, FLJ11164, TMPRSS2, TYROBP, G6PT1, PRIM1, GP5, DKFZP2566H073, RPS14, CCNG1, FANCG, CMAH, SORBS1, KIAA0800, C1QTNF3, UBCE71P5, FXR1, ZNF334, CNN2, RFC5, ACAA2, GNB1, FLJ22757, CDKN1C, UROD, KIAA1028, HD, CTSG, CLNS1A, P2RX1, TACC1, ADH5, RPL13A, ZNF363, PRKCH, AF020591, LOC51659, PER1, TFPI, TSN, BMI1, KIAA0625, MLLT2, TAF1C, DHFR, SLC23A1, HAGE, NAP1L4, EGFL3, SCA2, FLJ20489, SNAP25, USF2, CRYL1, GG2-1, EDN3, TRPC1, AP1S2, ERCC1, KIAA0582, RPL15, LOC54103, FLJ22557, CGI-127, CSNK2A2, ZNF278, EDG5, IPW, RASGRP2, SAE1, KIAA0725, RTN2, CTNS, FLJ20274, FLJ10276, LTBP4, FLJ10539, HYAL3, MTL5, MGEA6, BNIP3L, PARVB, MGC15523, KCNK7, IGHM, PASK, KIDINS220, PCM1, KIAA0092, ASB9, MAP3K4, CD1B, COL6A1, HCA127, ZNF262, GG2-1, CAPN3, SAP18, EIF3S5, ZNF337, EIF4A1, DBT, CROT, FLJ10474, FLJ10483, CBX8, DKFZP586M1523, CCRL1AP, FLJ14153, KIAA0397, COL2A1, CD164, TLE4, PRO2730, ATM, RFX5, KIAA0515, FLJ20542, HYPH, ERG-1, DBH, SCML2, GNAO1, WDR13, GCA, FLJ23323, FLJ11362, CGBP, MGAT1, HMGB2, NDUFA6, KIAA0515, KIF13A, OPA1, BRD1, ATP2B4, PSME1, KIAA0931, HPS4, KIAA1966, DKFZP564J0123, DBY, HUMNPIIY20, MAT2A, D1-FB, FLJ20294, ADSL, CSTF2T, ZNFN1A1, LOC51194, FLJ21269, DJ79P11.1, BCAT1, MGC21854, DKFZP586D0824, EMCN, C21orf91, SDPR, PRO1635, ITGA4, FLJ20171, ROBO4, ZNF6, DRLM, TAGAP, PRDM16, ST6Ga1II, GNAI1, EHZF, MGC10966, ARHGAP9, HEMGN, GNG2, LOC83690, PTGS1, MGC41924, USP2, FLJ33069, CT2, C4ST3, PRAM-1, FLJ132122, SLC11A3, BIC, TNFSF13B, FLJ37080, FLJ35564, KIAA1913, CDH26, BCL11A, FLJ30046, MGC7036, DKFZP566NO34, RARA, C1orf21, PAG, SH2D3C, FLJ00026, ST1P-1, FLJ39957, KLHL6, VIK, FLJ134922, SHANK3, FLJ00026, PTPN22, HRB2, ZDHHC2, DKFZP566K1924, SYTL4, DACH, FLJ21986, EVIN2, GAB3, CYYR1, MMP28, EHZF, FLJ00058, LOC93589, KLF12, CLLD8, KIAA1218, MGC16179, HS3ST3B1, ARHGAP9, LOC144402, LOC114928, FLJ39370, PRKACB, MGC13105, Ells1, CGI-145, EPB41L5, RAB39B, LOC145553, HRB2, SDCCAG33, ARRB1, EEF1A1, MGC12992, BBX, DAP10, CMG2, GPR27, GBP5, FLJ20202, UCC1, RAD52B, KIAA1554, AKNA, TBXAS1, a1/3GTP, JAK3, B2M, MGC20496, CLLD8, ALEX3, FLJ21438, MJD, FLJ22570, AP1S2, TFDP2, P5CR2, C1orf21, KIAA1554, Evil, MGC8721, FACL5, CYSLTR1, CTSS, Rgr, NID67, FLJ32194, MGC45400, KIAA1789, DCP1B, MGC4251, CPXM, SMBP, PARVG, ESRRBL1, C6orf33, MGC20262, C6orf33, MGC27027, LOC51234, ZNF33A, RGS18, KIAA1607, TIGA1, HOXA7, NAALADASEL, ATP8B2, CLYBL, DKFZP727G051, KIAA1214, WHIP, IRF5, UBL5, KIAA1946, GLTSCR2, CMG2, OSM, KIAA0748, FLJ11113, FLJ112994, EROI-L(BETA), NUCB2, KIAA1337, DEF6, POLH, FLJ11712, LOC91526, TTYH2, ACRBP, MAML3, FLJ00012, C6orf37, MYH11, C9orf24, HNRPD, CCNDBP1, DKFZP434L0117, GPR114, ANKH, MGC13170, NOG, CXorf10, C1QTNF4, NAV1, RPIB9, DKFZp571K0837, SFXN1, KIAA1497, PHACS, PAPOLA, ELAC1, MDS006, FLJ14167, LOC136895, CGGBP1, MGC45962, CGI-85, AUTS2, FXYD5, FLJ32009, FGD3, HSAJ1454, GRP58, KIAA1954, ELD/OSA1, PRex1, MGC11324, FLJ90013, NIN283, HCA127, DKFZP564D1378, HMGB1, TRB@, MGC4796, ASE-1, YR-29, FLJ25476, CGI-67, STK33, SLC25A21, ZNFN1A1, DRLM, PP2135, STMN3, CAMK2G, MGC16169, DC6, GCNT1, PRO1635, STRIN, DLC1, DKFZp761D221, FLJ10656, ZNFN1A4, SENP7, MGC34827, MGC15619, FLJ32942, RPL28, FLJ00005, FLJ23462, DKFZp762L0311, FLJ30726, MGC3200, ARRB1, EIF3S7, HSA9761, FLJ11896, MGC10744, KIAA1309, WDR9, KIAA1587, MIR, FLJ12953, MGC12921, LOC130617, NAV1, HPSE, FLJ20085, KIAA1982, KCNK17, KIAA1495, LOC64744, AUTL1, LOC91689, SEPP1, PPP2CA, KHDRBS1, DREV1, MGC35274, SNRPE, LOC91689, KIAA0853, FLJ13215, TACC1, MGC20262, MGC17515, MGC40157, DKFZP572C163, PRPF8, HINT1, FUSIP1, MEF2D, C20orf24, TADA2L, NIN283, FS, HSPCO63, ALS2, NHP2L1, LGALS12, MGC10986, KIAA1871, DKFZP434A0131, KIAA1949, DTNBP1, GPHN, SUV39H2, BRD7, FLJ32001, HYPC, EEF2K, ESRRB, ZNF226, IL18BP, CSRP2BP, HEMGN, FOXP1, SGKL, FLJ11220, TRIM4, FLJ21918, KIAA1545, MGC2474, CDCA7, HSPC002, LOC115294, LOC119710, GTF3A, TAGAP, TCF7L2, FLJ22690, OAZIN, TRAP1, MGC42174, MGC9850, KIAA1632, HSU53209, BIVM, BAALC, WHSC1, C16orf5, KIAA1238, MRS2L, CGI-105, ZDHHC2, LOC143903, DKFZp762NO610, NSE1, OSBPL7, HAVCR2, ASAHL, KIAA1798, TLR4, MGC10946, PRex1, FLJ31340, TAHCCP1, C20orf141, FLJ20313, TAF9L, FRSB, PRKRA, P66, KIAA0141, RARA, BANP, FLJ00007, DTNBP1, LRP5, KIAA1337, MGC29667, WHSC1, MMP28, EVIN2, Cab45, CED-6, PTER, ZNFN2A1, NDP52, CHES1, KIAA1635, NFAT5, FLJ32332, HTRA3, MAP4K1, KIAA1337, AP1S2, FLJ23306, HP1-BP74, KIAA1218, BTBD4, DKFZp761F0118, MGC16703, BAZ2B, MU, FLJ13614, MYO15B, OAZIN, LOC92799, CANX, SUFU, KIAA1954, AGS3, LAPTM4A, HP1-BP74, FLJ23467, FLJ12892, MGC40042, KIAA1143, RPL11, LSR7, CENPJ, NY-REN-58, NRM, FLJ23563, WASF2, AMBP, NIP30, EIF2AK4, MGC15429, TTC7L1, NICN1, FXC1, FLJ20793, SOC, RPL13, HYPC, CLONE24945, MGC24663, TEM7R, FLJ14768, DKFZp667M2411, STARD9, FOXP1, ELP3, KIAA1337, CDA017, PPP6C, PAK1, FLJ10876, EPC1, ZNF397, C21orf63, KIAA1805, MIR, CYYR1, DKFZp564B0769, EPSTI1, MDM4, MGC23947, MGC14421, SDCCAG33, DKFZp7620076, LOC93109, STN2, HSMPP8, FLJ20265, LOC85028, MGC15435, 1-Sep, MGC41917, MSI2, Jade-1, IL17D, MGC2752, MATR3, PRKRA, DKFZp434C1714, MGC4415, DKFZP727C091, MY038, FLJ35453, FLJ30794, DJ462O23.2, FLJ90130, FLJ22283, EEF2, LOC155066, ATPAF1, FLJ23499, STAM2, LOC85028, FLJ21709, LOC51279, TRA@, JAM3, SIAT6, KIAA1453, EIF2S3, LSR7, ROCK1, DKFZP566I1024, FANCD2, MEF-2, MGC2664, MGC15548, ZNF75A, HSPC126, EIF3S5, RBM7, FLJ20280, GSTA4, SEPP1, TIGD3, DKFZP434A1319, MCLC, MGC14136, DKFZP762N2316, LOC115330, D4ST-1, UCP4, PRMT6, LAK, NIN, FLJ10997, RAB4B, LMO4, RRN3, CENPH, FLJ23277, GBTS1, FLJ90013, LOC115509, PP2135, FLJ36175, SPINO, PAIP2, DKFZp761G0122, ATF71P, WBP1, MGC29937, MGC9564, CASP2, TIGD7, C4S-2, MGC25181, LOC89887, KIAA1387, FLJ22283, GIT2, MIR, SSBP3, LOC159090, U5-200KD, FLJ10997, ZNF295, PGBD1, HEL308, POLH, AP3M1, NORE1, SEMA6D, PPID, CUL5, LOC91663, FLJ13171, BAT4, RPLP1, KIAA1630, CT2, HSPC182, HMGB1, FLJ20280, FKBP5, EIF3S6, C15orf15, TRPC7, FLJ31153, TA-KRP, MGC17919, AP2A1, C20orf132, SECP43, PPIL2, FLJ14494, YARS, MGC10974, CLN6, C20orf81, U2AF1, KIAA1238, FLJ23861, LOC144455, DKFZp564D177, NIP30, TBC1D1, ZNF265, and PPP4R2.

While the above listed names are gene names, it is noted that the present invention contemplates the use of both the nucleic acid sequences as well as the peptides encoded thereby, as well as fragments of the nucleic acid and peptides, in the therapeutic and diagnostic methods and compositions of the present invention. The present invention is not limited in any way to the use of these particular gene signatures. Any combination of one or more markers that provides useful information can be used in the methods of the present invention. Any suitable marker that correlates with cancer or the progression of cancer can be utilized. Additional markers are also contemplated to be within the scope of the present invention. Any suitable method can be utilized to identify and characterize cancer stem cell markers suitable for use in the methods of the present invention.

In some embodiments, the present invention provides methods for detection of expression of cancer stem cell markers. In some embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine). In some embodiments, the presence of a marker on a cancer stem cell is used to provide a prognosis to a subject based on the stage-specific information on stem cell carcinogenesis. The information provided is also used to direct the course of treatment. For example, if a subject is found to have a marker indicative of a cancer stem cell at a particular stage of stem cell carcinogenesis, appropriate therapies can be started at an appropriate time to increase the efficacy.

### X. Multi-Stage Stem Cell Carcinogenesis (MSCC)

One aspect of the present invention includes the compositions and methods of generating the multi-stage stem cell carcinogenesis (MSCC) system. Transformation of a normal stem cell can arise at different stages of stem cell differentiation and carcinogenesis, for example, at the stage of ESC or lineage specific stem cell, or at the stage of multi-potent progenitor cells. Cancer stem cells generated therefrom exhibit distinct properties.

One embodiment of the invention includes methods of generating cancer stem cell lines at different stages of stem cell carcinogenesis by exposing cells to one or more rounds of mutagenesis, as described in details in Example 1. Once the stem cell clones are isolated and expanded to obtain multiple freezes of the same passage, extensive characterization is performed to obtain their genetic and cellular profiles. The characterization includes, but is not limited to, evaluation of the expression of pluripotency and differentiation markers as listed herein, expression of telomerase activity, proliferation capacity (BrdU incorporation), cell population doubling time, etc. To evaluate the level of genomic stability of the obtained lines, each cell clone is evaluated by karyotyping (G-banding) at the time of freezing and after 5 and 10 passages. Detailed methods are described in Example 2. The aforementioned techniques for characterizing the stem cell lines are well known to a person skilled in the relevant art.

The present invention also embodies classifying tumors based upon cancer stem cell profiles by comparing the gene expression pattern of a cancer stem cell to that of a normal untransformed stem cell counterpart or its normal derivatives that have been derived at the same stage as the cancer stem cell. Epigenetic changes of stem cell carcinogenesis can be assessed via the methods of the present invention. The term epigenetics typically refers to changes in phenotype or gene expression caused by mechanisms other than changes in the underlying DNA sequence. These changes may remain through cell divisions for the remainder of the cell's life and may also last for multiple generations. However, there is no change in the underlying DNA sequence of the organism (Adrian Bird (2007) Nature 447: 396-398), instead, non-genetic factors cause the organism's genes to express differently. One example of epigenetic changes in eukaryotic biology is the process of cellular differentiation. During morphogenesis, totipotent stem cells become the various pluripotent cell lines of the embryo which in turn become fully differentiated cells. In some embodiments, epigenetic changes including but not limited to microRNA profiles, DNA and histone methylation or modification are monitored and compared via the methods of the present invention. This invention further provides gene expression signatures of cancer stem cells derived at each different stage of stem cell carcinogenesis. Such gene expression profiles are predictors of tumor progression, distant metastases, likelihood of relapse, and survival. Genetic profiles based on the detection of differentially expressed polynucleotides and/or proteins that comprise a cancer stem cell when compared to a normal stem cell counterpart at various stages of malignant transformation can be used to predict clinical course and sensitivity to chemotherapeutic agents, guide selection of appropriate therapy, and monitor treatment response. Furthermore, methods of developing therapeutics targeting such cancer stem cell markers relative to their normal counterparts are provided herein. It is thought that once cancer stem cells are eliminated, the rest of the tumor will eventually die due to cell differentiation and cell death. Thus, therapeutics targeting stage-specific cancer stem cells over the normal stem cell counterparts as described herein allow for cancer stem cell-specific treatment that is stage-relevant and more effective at preventing tumor progression and tumor relapse.

The present invention employs methods for clustering genes into gene expression profiles by determining their expression levels in two different cell or tissue samples. The invention further envisions comparing cancer stem cell gene signature at various stages of malignant transformation to predict clinical outcome including, for example, metastasis and survival. Transcriptional profiles of the established cancer stem cell lines can be compared between tumor stem cells and normal stem cells at the same passage, non-tumorigenic tumor cells and normal stem cells, and tumor stem cells and non-tumorigenic tumor cells. These broad gene expression profiles can then be further refined, filtered, and subdivided into gene signatures based on various different criteria including, but not limited to, fold expression change, statistical analyses, correlation with expression of other genes, biological function (e.g. cell cycle regulators, transcription factors, proteases, etc.), clinical parameters, known therapeutic targets, identified expression in additional patient samples, and ability to predict clinical outcome.

In some embodiments, genomic status of the cell lines is assessed at multiple passages and at the time of banking by karyotyping with G-banding and array CGH analysis. Cell lines that exhibit high variability between passages in either phenotype or genotype are eliminated. In some embodiments, 2-3 stable cell lines that exhibit fully developed CSC phenotype and 2-3 less aggressive cell lines are selected from each genetic background for further analysis. Transcribed RNAs are analyzed by massively parallel sequencing and/or gene arrays. DNA and histone methylation can be assessed by Reduced Representation Bisulfite Sequencing and ChIP Sequencing, respectively. Results can be confirmed by RT-PCR and methylation specific PCR. The various molecular techniques including sequencing and PCR techniques are well known in the art (Molecular Cloning: A Laboratory Manual).

In some embodiments, bioinformatic analysis with network topology assessment is employed to identify genomic regions that harbor differences in expression profile, sequence variation or methylation pattern relative to control hESC and to uncover global molecular signatures associated with CSC phenotypes. Candidates with potential therapeutic values can be further selected for functional analysis.

In some embodiments, genes and/or pathways associated with CSC phenotypes can be assessed with an agent including but not limited to siRNA, small molecule inhibitor, and/or blocking antibody to determine whether these genes/pathways are necessary for maintenance of the CSC phenotype. In other embodiments, wildtype or mutated genes are expressed in normal hESC to assess whether their presence is sufficient to induce CSC phenotype.

### XI. Systems Biology Database

In some embodiments, a computer-based analysis program is used to translate the raw data generated by the biological assays into data of predictive value for a researcher and a clinician. Cancer stem cell lines generated at various stages of malignant transformation are extensively characterized. The methods of the present invention capture cells at both early and late stages of transformation and compare them to the normal isogenic stem cells and their derivatives. In some embodiments, the present invention incorporates into the MSCC model normal tissue-specific stem cells and progenitor cells that can be differentiated from hESC. The obtained data from cell characterization and pattern analysis as described herein are used to create a database and predictive mathematical model of stage-specific stem cell malignant transformation. Information on the major molecules and genetic pathways involved in stem cell transitions within this continuum is also provided by such database. For instance, the growths of the abnormal cells from their normal counterparts can be denoted with specific mutation probabilities. Such a system has been employed to predict that repeated insult to mature cells increases the formation of abnormal progeny, and hence the risk of cancer (Ganguly R. and Puri I. K. (2006) Cell Prolif 39:3-14). The clinician can access the predictive data using any suitable means. Thus, in some embodiments, the present invention provides a database in which the data are presented in a format suitable for interpretation by a treating clinician to predict disease course and devise the most effective therapy. The database also provides information suitable for research use. For example, the data can be used to further optimize the inclusion or elimination of cancer stem cell markers as useful indicators of a particular condition or stage of cancer. The methods of the present invention can be used to develop new in vitro and in vivo drug discovery platform that will identify specific molecular CSC drug targets and potential drug candidates.

### XII. Screening Anti-Cancer Therapeutics:

In some embodiments, the present invention provides drug screening assays (e.g., to screen for anticancer drugs). The screening methods of the present invention utilize cancer stem cell markers that are differentially expressed at different stages of malignant transformation and also markers that differentiate a cancer stem cell from a normal non-cancer stem cell or its derivatives. For example, in some embodiments, the present invention provides methods of screening for compound that alter (e.g., increase or decrease) the expression of cancer stem cell genes. In some embodiments, candidate compounds are small molecules, antibodies, or RNA agents directed against the cancer stem cell markers. In other embodiments, candidate compounds are directed against molecules that are preferentially expressed on a cancer stem cell relative to a normal non-cancer stem cell or its derivatives. In certain embodiments, libraries of compounds of small molecules are screened using the methods described herein.

In one screening method, candidate compounds are evaluated for their ability to alter cancer stem cell marker expression by contacting a compound with a cell expressing a cancer stem cell marker and then assaying for the effect of the candidate compounds on expression. In some embodiments, the effect of candidate compounds on expression of a cancer stem cell marker is assayed by detecting the level of cancer marker mRNA expressed by the cell. mRNA expression can be detected by any suitable method. In other embodiments, the effect of candidate compounds on expression of cancer stem cell genes is assayed by measuring the level of polypeptide encoded by the cancer markers. The level of polypeptide expressed can be measured using any suitable method known in the art.

Specifically, the present invention provides screening methods for identifying modulators, i.e., candidate or test compounds or agents (e.g., proteins, peptides, peptidomimetics, peptoids, small molecules or other drugs) which bind to, or alter the signaling or function associated with the cancer stem cell markers of the present invention, have a stimulatory or inhibitory effect on, for example, cancer stem cell marker expression or activity, or have a stimulatory or inhibitory effect on, for example, the expression or activity of a substrate of the cancer stem cell marker. Compounds thus identified can be used to modulate the activity of cancer stem cell marker genes either directly or indirectly in a therapeutic protocol, to modulate the biological function of the target gene product, or to identify compounds that disrupt normal target gene interactions. Compounds which inhibit the activity or expression of the cancer stem cell markers are useful in the treatment of proliferative disorders such as cancer, particularly metastatic cancer or, eliminating or controlling tumor stem cells to prevent or reduce the risk of cancer.

The invention provides assays for screening candidate or test compounds that are substrates of a cancer stem cell marker protein or polypeptide or a biologically active portion thereof. In another embodiment, the invention provides assays for screening candidate or test compounds that bind to or modulate the activity of a cancer stem cell marker protein or polypeptide or a biologically active portion thereof.

The test compounds of the present invention can be obtained using any of the numerous approaches in combinatorial library methods known in the art, including biological libraries; peptoid libraries; spatially addressable parallel solid phase or solution phase libraries; synthetic library methods requiring deconvolution; the one-bead one-compound library method; and synthetic library methods using affinity chromatography selection. The biological library and peptoid library approaches are for use with peptide libraries, while the other four approaches are applicable to peptide, non-peptide oligomer or small molecule libraries of compounds (Lam (1997) Anticancer Drug Des. 12:145).

In other embodiments of the present invention, a cancer stem cell gene signature, either representative of a particular stage or differentially expressed by cancer stem cells as compared to normal stem cells, can be used experimentally to test and assess lead compounds including, for example, small molecules, RNAs, and antibodies for the treatment of cancer. For example, tumor cells from a patient can be screened for expression of a particular cancer stem cell gene signature, and then transplanted into the xenograft model, and assess the effects of the test compounds, such as antibodies against one or more cancer stem cell markers described herein, on tumor growth and survival. Furthermore a cancer gene profile can be determined following treatment and the cancer gene profile compared to a cancer stem cell gene signature to assess the effectiveness of the therapy and in turn guide a future treatment regimen. In addition the efficacy of test compounds can be assessed against different tumor subclasses, e.g. tumors from different stages of malignancy. For example, test compounds can be used in xenografts of tumors that express a cancer gene profile that is highly correlated with a cancer stem cell signature of a particular stage of malignancy, or tumors having a gene profile that correlates with a cancer stem cell gene that is not expressed on normal stem cells, or tumors having a gene profile that does not correlate with a cancer stem cell gene signature. Any differences in response of the different tumor subclasses to the test compound are determined and used to optimize treatment for particular classes of tumors.

Agents that preferentially affect cancer stem cells of particular stages as described herein can be used to treat a variety of cancers including, but not limited to, biliary tract cancer; bladder cancer; brain cancer including glioblastomas and medulloblastomas; cervical cancer; choriocarcinoma; colon cancer; ondometial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymnphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and modullar carcinoma; and renal cancer including adenocarcinoma and Wilms tumor.

### XIII. Transgenic Cells and Non-Human Mammals

The present invention embodies the generation and use of transgenic animals comprising an exogenous cancer marker gene of the present invention or mutants and variants thereof or knock-outs thereof. In some embodiments, the transgenic animal displays an altered phenotype (e.g., increased or decreased presence of markers) as compared to wild-type animals. In some embodiments, the transgenic animals further display an increased or decreased growth of tumors or evidence of cancer. Exemplary transgenic non-human mammals include, but are not limited to, mice, rats, chickens, cows, and pigs. In certain examples, a transgenic non-human mammal has a knock-out of one or more of the target sequences associated with a gene involved in stem cell carcinogenesis, and/or has a differential level of expression and/or function in cancer stem cells as compared to normal untransformed stem cells. Such knock-out animals are useful for studying the stages of stem cell carcinogenesis and reducing or preventing the progression of cancer.

Expression of the sequence used to knock-out or functionally delete the desired gene can be regulated by choosing the appropriate promoter sequence. For example, constitutive promoters can be used to ensure that the functionally deleted gene is never expressed by the animal. In contrast, an inducible promoter can be used to control when the transgenic animal does or does not express the gene of interest. Exemplary inducible promoters include tissue-specific promoters and promoters responsive or unresponsive to a particular stimulus (such as light, oxygen, chemical concentration), including the tetracycline/doxycycoine regulated promoters (TET-off, TET-on), ecdysone-inducible promoter, and the Cre/loxP recombinase system.

In one embodiment a transgenic mouse with a gene involved in stem cell carcinogenesis or a disrupted endogenous gene involved in stem cell carcinogenesis, can be examined after exposure to various mutagens and/or carcinogens, such as ICR191, to determine the resistance or susceptibility of such animal to developing cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects are evaluated.

In an alternative embodiment a transgenic pig with a human gene or a disrupted endogenous gene involved in stem cell carcinogenesis, can be produced and used as an animal model to determine susceptibility to cancer development. Transgenic animals, including methods of making and using transgenic animals, are well known to one skilled in the art.

### XIV. Methods of Detecting Cancer

Conventional cancer screening methods include, among others: physical exam, mammography (breast cancer), PAP smear (cervical cancer), stool guaiac and colonoscopy (colon cancer), PSA (prostate cancer), skin suveillance (skin cancer), sputum analysis and chest X-ray (lung cancer), among others. For a positive test result, however, all of these methods unfortunately require that a significant number of cancer cells be present in the examined patient. Accordingly, such “early” detection methods are often too late. For example, approximately 30% of patients with “early” breast cancer without gross evidence of lymphatic spread who have undergone “complete” surgical resection will still develop recurrent disease at a later date. In addition, radiographically-detectable lung tumors are often incurable despite their seemingly “small” size. These disturbing findings indicate that conventional detection techniques do not identify cancer early enough.

By the multi-stage stem cell carcinogenesis (MSCC) system, normal stem cells are induced to mutate via one or more rounds of mutagenesis to become premalignant and malignant cells that represent different stages of stem cell carcinogenesis. At each stage of the stem cell carcinogenesis continuum including the normal untransformed stage, a batch of the generated stem cells is saved as a control for subsequent profiling. The generated cancer stem cell lines together with their normal untransformed counterparts are subjected to extensive characterization including, but not limited to, change in expression of a gene, enzymatic activity, telomerase activity, genomic stability, chromosomal modifications, mutations, epigenetic changes, cell growth, cell differentiation, cell polarization, cell adhesion, cell migration, and tissue invasiveness. The profiles of these cancer stem cells that represent different stages of malignant transformation are stored and used to generate a database, which provides predictive and diagonostic information with important clinical value.

Neoplastic stem cells could arise as an aberrancy at any point in the development of a given tissue e.g., during adult tissue genesis or even as early as embryogenesis when tissues are being formed. Using MSCC system is a novel and extremely sensitive method for the early detection of cancer. When a stem cell sample, either embryonic or adult stem cell, is obtained from a subject including a human, such stem cell sample is subjected to the same type and level of characeterization as used in the MSCC system. The subject mentioned herein can be a healthy person, a person at risk of developing a cancer, an asymptomatic person who has not been diagnosed with cancer but has premalignant cells, or a person who has already been diagnosed with cancer. The profile of the stem cell sample is subsequently compared to the existing profiles of the stem cell lines stored in the MSCC database. Based on the information given by the MSCC database, a clinician can make predictive and diagnostic recommendations to the subject. Appropriate prophylactic and/or therapeutic measurements can then be devised based on the profile comparison. Therapeutic measurements include, but are not limited to, conventional therapies such as surgical excision, irradiation, chemotherapy, either alone or in combination, with an agent that preferentially targets cancer stem cells over normal stem cells as described herein.

Early detection methods using the MSCC system should in theory allow assessment of a cancer prophecy at an early age, e.g. at birth or even possibly in utero. Adult-onset tumors can also be detected much earlier by MSCC system-based methods than by conventional means. Such an MSCC system-based method for early cancer detection can be used in patients at risk for developing cancer (e.g., because of family history or environmental risks, such as job hazards or smoking) or in patients in clinical remission from cancer. In addition, MSCC system-based methods could also be used in routine cancer screening of the asymptomatic patient.

### XV. Methods of Treatment: Anti-Cancer Therapy

One embodiment of the present invention relates to methods of using pharmaceutical compositions and kits comprising agents that preferentially have effect on a cancer stem cell relative to a non-cancer stem cell or its derivatives. Another embodiment of the present invention provides methods, pharmaceutical compositions, and kits for the treatment of animal subjects. The term “animal subject” as used herein includes humans as well as other mammals. The term “treating” as used herein includes achieving a therapeutic benefit and/or a prophylactic benefit. By therapeutic benefit is meant eradication or amelioration of the cancer. Also, a therapeutic benefit is achieved with the eradication or amelioration of one or more of the physiological symptoms associated with cancer such that an improvement is observed in the animal subject, notwithstanding the fact that the animal subject may still be afflicted with that cancer.

The current invention also includes candidate therapeutic agents that preferentially affect cancer stem cells relative to normal stem cells. In some embodiments, the present invention embodies compositions comprising oligomeric antisense compounds, particularly oligonucleotides, for use in modulating the function of nucleic acid molecules encoding markers preferentially expressed on cancer stem cells relative to their normal counterparts. This is accomplished by providing antisense compounds that specifically hybridize with one or more nucleic acids encoding such cancer markers of the present invention. The overall effect of antisense interference is modulation of the expression of such cancer stem cell markers of the present invention. In the context of the present invention, “modulation” means either an increase (stimulation) or a decrease (inhibition) in the expression of a gene. For example, expression can be inhibited to potentially prevent tumor growth.

The present invention also embodies the use of any genetic manipulation to modulate the expression of a cancer stem cell cancer marker of the present invention. One embodiment is gene knockout (e.g., removing the cancer stem cell gene from the chromosome using, for example, recombination), or addition of a heterologous gene (e.g. controlled by an inducible promoter), and the like. Delivery of nucleic acid construct to cells in vitro or in vivo can be conducted using any suitable method known in the art.

In some embodiments, the present invention provides an antibody-based agent targeting a cancer stem cell marker expressed on a tumor at a particular stage of malignancy, or a cancer stem cell marker that is preferentially expressed on a cancer stem cell relative to a normal untransformed stem cell. The antibody-based agent in any suitable form of an antibody e.g., monoclonal, polyclonal, or synthetic, can be utilized in the therapeutic methods disclosed herein. The antibody-based agents include any binding fragment of an antibody and also peptibodies, which are engineered therapeutic molecules that can bind to human drug targets and contain peptides linked to the constant domains of antibodies. In some embodiments, the antibodies used for cancer therapy are humanized antibodies. Methods for humanizing antibodies are well known in the art. In another embodiment, the therapeutic antibodies comprise an antibody generated against a cancer stem cell marker of the present invention, wherein the antibody is conjugated to a cytotoxic agent.

The present invention also embodies the use of any pharmacologic agent that can be conjugated to an antibody or an antibody binding fragment, and delivered in active form. Examples of such agents include cytotoxins, radioisotopes, hormones such as a steroid, anti-metabolites such as cytosines, and chemotherapeutic agents. Any suitable chemotherapeutic agent can be used in combination with the antibody-based agent targeting a cancer stem cell marker described herein. Other embodiments can include agents such as a coagulant, a cytokine, growth factor, bacterial endotoxin or a moiety of bacterial endotoxin. The targeting antibody-based agent directs the toxin to, and thereby selectively modulates the cell expressing the targeted cancer stem cell marker. In some embodiments, therapeutic antibodies employ cross-linkers that provide high in vivo stability (Thorpe et al., Cancer Res., 48:6396, 1988). In any event, it is proposed that agents such as these can, if desired, be successfully conjugated to an antibody or an antibody binding fragment, in a manner that will allow their targeting, internalization, release or presentation at the site of the targeted cells expressing the enzyme as required using known conjugation technology. In other embodiments, RNAi is used to regulate expression of the cancer stem cell markers of the present invention.

For embodiments where a prophylactic benefit is desired, a pharmaceutical composition of the agent described in this invention may be administered to a patient at risk of developing cancer, or metastasis, or tumor relapse, or to a patient reporting one or more of the physiological symptoms of a cancer, even though a diagnosis of the condition may not have been made. Administration may prevent the tumor from developing, metastasizing, and/or relapsing. Or it may reduce, lessen, shorten and/or otherwise ameliorate the cancer that develops.

In yet other embodiments, the present invention provides kits for the detection and characterization of cancer. In some embodiments, the kits contain antibodies specific for a cancer stem cell marker, in addition to detection reagents and buffers. In other embodiments, the kits contain reagents specific for the detection of mRNA or cDNA (e.g., oligonucleotide probes or primers). In some embodiments, the kits contain all of the components necessary and/or sufficient to perform a detection assay, including all controls, directions for performing assays, and any necessary software for analysis and presentation of results.

The present invention also includes kits that can be used to treat cancer. These kits comprise an agent or combination of agents that preferentially affect or modulate cancer stem cells relative to their normal stem cell counterparts. Such kits may also include information, such as scientific literature references, package insert materials, clinical trial results, and/or summaries of these and the like, which indicate or establish the activities and/or advantages of the agent. Such information may be based on the results of various studies, for example, studies using experimental animals involving in vivo systems and studies based on human clinical trials. Kits described herein can be provided, marketed and/or promoted to health providers, including physicians, nurses, pharmacists, formulary officials, and the like.

### XVI. Pharmaceutical Compositions, Formulations, Administration and Dosage

Yet another aspect of the present invention relates to formulations, routes of administration and effective doses for pharmaceutical compositions comprising an agent (e.g., a small molecule, antisense, antibody, or siRNA) that preferentially targets cancer stem cells relative to normal stem cells as described in the present invention. Such pharmaceutical compositions can be used to treat various types of cancer as described herein.

Compounds of the invention may be administered as pharmaceutical formulations including those suitable for oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. General information on drug delivery system's can be found in Ansel et al., Pharmaceutical Dosage Forms and Drug Delivery Systems (Lippencott Williams & Wilkins, Baltimore Md. (1999).

In various embodiments, the pharmaceutical composition includes carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In some embodiments, the pharmaceutical preparation is substantially free of preservatives. In other embodiments, the pharmaceutical preparation may contain at least one preservative. General methodology on pharmaceutical dosage forms is found in Ansel et al., Pharmaceutical Dosage Forms and Drug Delivery Systems (Lippencott Williams & Wilkins, Baltimore Md. (1999)). It will be recognized that, while any suitable carrier known to those of ordinary skill in the art may be employed to administer the compositions of this invention, the type of carrier will vary depending on the mode of administration.

Compounds may also be encapsulated within liposomes using well-known technology. Biodegradable microspheres may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

The compound may be administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 2.sup.87-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

The concentration of drug may be adjusted, the pH of the solution buffered and the isotonicity adjusted to be compatible with intravenous injection, as is well known in the art.

The compounds of the invention may be formulated as a sterile solution or suspension, in suitable vehicles, well known in the art. The pharmaceutical compositions may be sterilized by conventional, well-known sterilization techniques, or may be sterile filtered. The resulting aqueous solutions may be packaged for use as is, or lyophilized, the lyophilized preparation being combined with a sterile solution prior to administration. Suitable formulations and additional carriers are described in Remington “The Science and Practice of Pharmacy” (20^{th} Ed., Lippincott Williams & Wilkins, Baltimore Md.), the teachings of which are incorporated by reference in their entirety herein.

The agents or their pharmaceutically acceptable salts may be provided alone or in combination with one or more other agents or with one or more other forms. For example a formulation may comprise one or more agents in particular proportions, depending on the relative potencies of each agent and the intended indication. For example, in compositions for targeting two different host targets, and where potencies are similar, about a 1:1 ratio of agents may be used. The two forms may be formulated together, in the same dosage unit e.g., in one cream, suppository, tablet, capsule, aerosol spray, or packet of powder to be dissolved in a beverage; or each form may be formulated in a separate unit, e.g., two creams, two suppositories, two tablets, two capsules, a tablet and a liquid for dissolving the tablet, two aerosol sprays, or a packet of powder and a liquid for dissolving the powder, etc.

The term “pharmaceutically acceptable salt” means those salts which retain the biological effectiveness and properties of the agents used in the present invention, and which are not biologically or otherwise undesirable. For example, a pharmaceutically acceptable salt does not interfere with the beneficial effect of an agent of the invention in preventing or treating cancer by preferentially modulating a cancer stem cell relative to a normal untransformed stem cell.

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the agent(s) contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

A pharmaceutically acceptable ester or amide refers to those which retain biological effectiveness and properties of the agents used in the present invention, and which are not biologically or otherwise undesirable. For example, the ester or amide does not interfere with the beneficial effect of an agent of the invention in preventing or treating cancer by preferentially modulating a cancer stem cell relative to a normal untransformed stem cell. Typical esters include ethyl, methyl, isobutyl, ethylene glycol, and the like. Typical amides include unsubstituted amides, alkyl amides, dialkyl amides, and the like.

In some embodiments, an agent may be administered in combination with one or more other compounds, forms, and/or agents, e.g., as described above. Pharmaceutical compositions comprising combinations of an anti-cancer drug with one or more other active agents can be formulated to comprise certain molar ratios. For example, molar ratios of about 99:1 to about 1:99 of an anti-cancer stem cell agent to the other active agent can be used. In some subset of the embodiments, the range of molar ratios of the anti-cancer stem cell agent:other active agent is selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio of anti-cancer stem cell agent:other active agent may be about 1:9, and in some embodiments may be about 1:1. The two agents, forms and/or compounds may be formulated together, in the same dosage unit e.g., in one cream, suppository, tablet, capsule, or packet of powder to be dissolved in a beverage; or each agent, form, and/or compound may be formulated in separate units, e.g., two creams, suppositories, tablets, two capsules, a tablet and a liquid for dissolving the tablet, an aerosol spray a packet of powder and a liquid for dissolving the powder, etc. If necessary or desirable, the agents and/or combinations of agents may be administered with still other agents. The choice of agents that can be co-administered with the agents and/or combinations of agents of the instant invention can depend, at least in part, on the condition being treated. Agents of particular use in the formulations of the present invention include, for example, any agent having a therapeutic effect for cancer.

In some alternative embodiments for the treatment of cancer formulations of the instant invention may additionally contain one or more conventional anti-cancer agents including, but are not limited to, nitrosourea-related compounds, lomustine, carmustine, streptozocin, mechlorethamine, melphalan, uracil nitrogen mustard, chlorambucil, cyclophosphamide, iphosphamide, alkylating agents such as cisplatin, carboplatin, mitomycin, thiotepa, dacarbazin, procarbazine, hexamethyl melamine, triethylene melamine, busulfan, pipobroman, mitotane; antimetabolites such as methotrexate, trimetrexate, a methotrexate analog, pentostatin, cytarabine, ara-CMP, an activated form of cytarabine, fludarabine phosphate, hydroxyurea, fluorouracil, floxuridine, chlorodeoxyadenosine, gemcitabine, thioguanine, 6-Mercaptopurine; bleomycin, topoisomerase I poisons such as topotecan, irinotecan, camptothecin sodium salt, a strucutral analog of topotecan and irinotecan; topoisomerase II poisons such as daunorubicin, doxorubicin, idarubicin, mitoxantrone, teniposide, etoposide; dactinomycin, mithramycin; spindle poisons such as vinblastine, vincristine, navelbine, paclitaxel, and docetaxel.

The agent(s) (or pharmaceutically acceptable salts, esters or amides thereof) may be administered per se or in the form of a pharmaceutical composition wherein the active agent(s) is in an admixture or mixture with one or more pharmaceutically acceptable carriers. A pharmaceutical composition, as used herein, may be any composition prepared for administration to a subject. Pharmaceutical compositions for use in accordance with the present invention may be formulated in conventional manner using one or more physiologically acceptable carriers, comprising excipients, diluents, and/or auxiliaries, e.g., which facilitate processing of the active agents into preparations that can be administered. Proper formulation may depend at least in part upon the route of administration chosen. The agent(s) useful in the present invention, or pharmaceutically acceptable salts, esters, or amides thereof, can be delivered to a patient using a number of routes or modes of administration, including oral, buccal, topical, rectal, transdermal, transmucosal, subcutaneous, intravenous; and intramuscular applications, as well as by inhalation.

For oral administration, the agents can be formulated readily by combining the active agent(s) with pharmaceutically acceptable carriers well known in the art. Such carriers enable the agents of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Generally, the agents of the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use may contain agent(s) of this invention with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents may be required to bring the agents into solution, due to, for example, the presence of large lipophilic moieties. Alternatively, emulsions, suspensions, or other preparations, for example, liposomal preparations, may be used. With respect to liposomal preparations, any known methods for preparing liposomes for treatment of a condition may be used. See, for example, Bangham et al., J. Mol. Biol. 23: 238-252 (1965) and Szoka et al., Proc. Natl. Acad. Sci. USA 75: 4194-4198 (1978), incorporated herein by reference. Ligands may also be attached to the liposomes to direct these compositions to particular sites of action. Agents of this invention may also be integrated into foodstuffs, e.g., cream cheese, butter, salad dressing, or ice cream to facilitate solubilization, administration, and/or compliance in certain patient populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The agents may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Suitable fillers or carriers with which the compositions can be administered include agar, alcohol, fats, lactose, starch, cellulose derivatives, polysaccharides, polyvinylpyrrolidone, silica, sterile saline and the like, or mixtures thereof used in suitable amounts. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

When formulating compounds of the invention for oral administration, it may be desirable to utilize gastroretentive formulations to enhance absorption from the gastrointestinal (G1) tract. A formulation which is retained in the stomach for several hours may release compounds of the invention slowly and provide a sustained release that may be preferred in some embodiments of the invention. Disclosure of such gastro-retentive formulations are found in Klausner, E. A.; Lavy, E.; Barta, M.; Cserepes, E.; Friedman, M.; Hoffman, A. 2003 “Novel gastroretentive dosage forms: evaluation of gastroretentivity and its effect on levodopa in humans.” Pharm. Res. 20, 1466-73, Hoffman, A.; Stepensky, D.; Lavy, E.; Eyal, S. Klausner, E.; Friedman, M. 2004 “Pharmacokinetic and pharmacodynamic aspects of gastroretentive dosage forms” Int. J. Pharm. 11, 141-53, Streubel, A.; Siepmann, J.; Bodmeier, R.; 2006 “Gastroretentive drug delivery systems” Expert Opin. Drug Deliver. 3, 217-3, and Chavanpatil, M. D.; Jain, P.; Chaudhari, S.; Shear, R.; Vavia, P. R. “Novel sustained release, swellable and bioadhesive gastroretentive drug delivery system for olfoxacin” Int. J. Pharm. 2006 epub March 24. Expandable, floating and bioadhesive techniques may be utilized to maximize absorption of the compounds of the invention.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol.

For injectable formulations, the vehicle may be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation may also comprise polymer compositions which are biocompatible, biodegradable, such as poly(lactic-co-glycolic)acid. These materials may be made into micro or nanospheres, loaded with drug and further coated or derivatized to provide superior sustained release performance. Vehicles suitable for periocular or intraocular injection include, for example, suspensions of therapeutic agent in injection grade water, liposomes and vehicles suitable for lipophilic substances. Other vehicles for periocular or intraocular injection are well known in the art.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

When administration is by injection, the active compound may be formulated in aqueous solutions, specifically in physiologically compatible buffers such as Hanks solution, Ringer's solution, or physiological saline buffer. The solution may contain formulatory agents such as suspending, stabilizing and/or dispersing agents. Alternatively, the active compound may be in powder form for constitution with a suitable vehicle, e.g., sterile pyrogen-free water, before use. In some embodiments, the pharmaceutical composition does not comprise an adjuvant or any other substance added to enhance the immune response stimulated by the peptide. In some embodiments, the pharmaceutical composition comprises a substance that inhibits an immune response to the peptide. Methods of formulation are known in the art, for example, as disclosed in Remington's Pharmaceutical Sciences, latest edition, Mack Publishing Co., Easton P.

In addition to the formulations described previously, the agents may also be formulated as a depot preparation. Such long acting formulations may be administered by implantation or transcutaneous delivery (for example subcutaneously or intramuscularly), intramuscular injection or use of a transdermal patch. Thus, for example, the agents may be formulated with suitable polymeric or hydrophobic materials (for example as an emulsion in an acceptable oil) or ion exchange resins, or as sparingly soluble derivatives, for example, as a sparingly soluble salt.

In some embodiments, pharmaceutical compositions comprising one or more agents of the present invention exert local and regional effects when administered topically or injected at or near particular sites of infection. Direct topical application, e.g., of a viscous liquid, solution, suspension, dimethylsulfoxide (DMSO)-based solutions, liposomal formulations, gel, jelly, cream, lotion, ointment, suppository, foam, or aerosol spray, may be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations may also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983. In some embodiments, local/topical formulations comprising an agent that preferentially modulates cancer stem cells are used to treat skin-related cancer.

Pharmaceutical compositions of the present invention may contain a cosmetically or dermatologically acceptable carrier. Such carriers are compatible with skin, nails, mucous membranes, tissues and/or hair, and can include any conventionally used cosmetic or dermatological carrier meeting these requirements. Such carriers can be readily selected by one of ordinary skill in the art. In formulating skin ointments, an agent or combination of agents of the instant invention may be formulated in an oleaginous hydrocarbon base, an anhydrous absorption base, a water-in-oil absorption base, an oil-in-water water-removable base and/or a water-soluble base. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

The compositions according to the present invention may be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (0/W or oil in water) or, conversely, (W/O or water in oil), microemulsions or alternatively microcapsules, microparticles or lipid vesicle dispersions of ionic and/or nonionic type. These compositions can be prepared according to conventional methods. Other than the agents of the invention, the amounts of the various constituents of the compositions according to the invention are those conventionally used in the art. These compositions in particular constitute protection, treatment or care creams, milks, lotions, gels or foams for the face, for the hands, for the body and/or for the mucous membranes, or for cleansing the skin. The compositions may also consist of solid preparations constituting soaps or cleansing bars.

Compositions of the present invention may also contain adjuvants common to the cosmetic Sand dermatological fields, such as hydrophilic or lipophilic gelling agents, hydrophilic or lipophilic active agents, preserving agents, antioxidants, solvents, fragrances, fillers, sunscreens, odor-absorbers and dyestuffs. The amounts of these various adjuvants are those conventionally used in the fields considered and, for example, are from about 0.01% to about 20% of the total weight of the composition. Depending on their nature, these adjuvants may be introduced into the fatty phase, into the aqueous phase and/or into the lipid vesicles.

In some embodiments, ocular types of cancer may be effectively treated with ophthalmic solutions, suspensions, ointments or inserts comprising an agent or combination of agents of the present invention. Eye drops may be prepared by dissolving the active ingredient in a sterile aqueous solution such as physiological saline, buffering solution, etc., or by combining powder compositions to be dissolved before use. Other vehicles may be chosen, as is known in the art, including but not limited to: balance salt solution, saline solution, water soluble polyethers such as polyethyene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

The solubility of the components of the present compositions may be enhanced by a surfactant or other appropriate co-solvent in the composition. Such cosolvents include polysorbate 20, 60, and 80, Pluronic F68, F-84 and P-103, cyclodextrin, or other agents known to those skilled in the art. Such co-solvents may be employed at a level of from about 0.01% to 2% by weight.

The compositions of the invention may be packaged in multidose form. Preservatives may be preferred to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In the prior art ophthalmic products, such preservatives may be employed at a level of from 0.004% to 0.02%. In the compositions of the present application the preservative, preferably benzalkonium chloride, may be employed at a level of from 0.001% to less than 0.01%, e.g. from 0.001% to 0.008%, preferably about 0.005% by weight. It has been found that a concentration of benzalkonium chloride of 0.005% may be sufficient to preserve the compositions of the present invention from microbial attack.

In some embodiments, cancer of the ear can be effectively treated with otic solutions, suspensions, ointments or inserts comprising an agent or combination of agents of the present invention.

In some embodiments, the agents of the present invention are delivered in soluble rather than suspension form, which allows for more rapid and quantitative absorption to the sites of action. In general, formulations such as jellies, creams, lotions, suppositories and ointments can provide an area with more extended exposure to the agents of the present invention, while formulations in solution, e.g., sprays, provide more immediate, short-term exposure.

In some embodiments relating to topical/local application, the pharmaceutical compositions can include one or more penetration enhancers. For example, the formulations may comprise suitable solid or gel phase carriers or excipients that increase penetration or help delivery of agents or combinations of agents of the invention across a permeability barrier, e.g., the skin. Many of these penetration-enhancing compounds are known in the art of topical formulation, and include, e.g., water, alcohols (e.g., terpenes like methanol, ethanol, 2-propanol), sulfoxides (e.g., dimethyl sulfoxide, decylmethyl sulfoxide, tetradecylmethyl sulfoxide), pyrrolidones (e.g., 2-pyrrolidone, N-methyl-2-pyrrolidone, N-(2-hydroxyethyl)pyrrolidone), laurocapram, acetone, dimethylacetamide, dimethylformamide, tetrahydrofurfuryl alcohol, L-α-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamethylene lauramide, proteolytic enzymes, a-bisabolol, d-limonene, urea and N,N-diethyl-m-toluamide, and the like. Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. In some embodiments, the pharmaceutical compositions will include one or more such penetration enhancers.

In some embodiments, the pharmaceutical compositions for local/topical application can include one or more antimicrobial preservatives such as quaternary ammonium compounds, organic mercurials, p-hydroxy benzoates, aromatic alcohols, chlorobutanol, and the like.

Gastrointestinal cancers can be effectively treated with orally- or rectally delivered solutions, suspensions, ointments, enemas and/or suppositories comprising an agent or combination of agents of the present invention.

Respiratory cancers can be effectively treated with aerosol solutions, suspensions or dry powders comprising an agent or combination of agents of the present invention. Administration by inhalation is particularly useful in treating cancers of the lung. The aerosol can be administered through the respiratory system or nasal passages. For example, one skilled in the art will recognize that a composition of the present invention can be suspended or dissolved in an appropriate carrier, e.g., a pharmaceutically acceptable propellant, and administered directly into the lungs using a nasal spray or inhalant. Aerosol formulations may contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that the agent or combination of agents of the present invention is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants useful in the present invention include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as the ethers. An aerosol formulation of the invention can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present invention can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. The solvent can be used to dissolve the agent and/or retard the evaporation of the propellant. Solvents useful in the invention include, for example, water, ethanol and glycols. Any combination of suitable solvents can be use, optionally combined with preservatives, antioxidants, and/or other aerosol components. An aerosol formulation can also be a dispersion or suspension. A suspension aerosol formulation may comprise a suspension of an agent or combination of agents of the instant invention, e.g., an agent that preferentially modulates cancer stem cells. Dispersing agents useful in the invention include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can similarly be formulated as an emulsion. An emulsion aerosol formulation can include, for example, an alcohol such as ethanol, a surfactant, water and a propellant, as well as an agent or combination of agents of the invention, e.g., an agent that preferentially modulates cancer stem cells. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

The compounds of the invention may be formulated for administration as suppositories. A low melting wax, such as a mixture of triglycerides, fatty acid glycerides, Witepsol S55 (trademark of Dynamite Nobel Chemical, Germany), or cocoa butter is first melted and the active component is dispersed homogeneously, for example, by stirring. The molten homogeneous mixture is then poured into convenient sized molds, allowed to cool, and to solidify.

The compounds of the invention may be formulated for vaginal administration. Pessaries, tampons, creams, gels, pastes, foams or sprays containing in addition to the active ingredient such carriers as are known in the art to be appropriate.

It is envisioned additionally, that the compounds of the invention may be attached releasably to biocompatible polymers for use in sustained release formulations on, in or attached to inserts for topical, intraocular, periocular, or systemic administration. The controlled release from a biocompatible polymer may be utilized with a water soluble polymer to form a instillable formulation, as well. The controlled release from a biocompatible polymer, such as for example, PLGA microspheres or nanospheres, may be utilized in a formulation suitable for intra ocular implantation or injection for sustained release administration. Any suitable biodegradable and biocompatible polymer may be used.

Pharmaceutical compositions suitable for use in the present invention include compositions wherein the active ingredients are present in an effective amount, i.e., in an amount effective to achieve therapeutic and/or prophylactic benefit in a host with cancer. The actual amount effective for a particular application will depend on the condition or conditions being treated, the condition of the subject, the formulation, and the route of administration, as well as other factors known to those of skill in the art. Determination of an effective amount of an agent that preferentially affects cancer stem cells is well within the capabilities of those skilled in the art, in light of the disclosure herein, and will be determined using routine optimization techniques.

The effective amount for use in humans can be determined from animal models. For example, a dose for humans can be formulated to achieve circulating, liver, topical and/or gastrointestinal concentrations that have been found to be effective in animals. One skilled in the art can determine the effective amount for human use, especially in light of the animal model experimental data described herein. Based on animal data, and other types of similar data, those skilled in the art can determine the effective amounts of compositions of the present invention appropriate for humans.

The effective amount when referring to an agent or combination of agents of the invention will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

Further, appropriate doses for an agent that preferentially modulates cancer stem cells relative to normal stem cells can be determined based on in vitro experimental results. For example, the in vitro potency of an agent in inhibiting a cancer stem cell relative to a normal untransformed stem cell provides information useful in the development of effective in vivo dosages to achieve similar biological effects.

In some embodiments, administration of agents of the present invention may be intermittent, for example administration once every two days, every three days, every five days, once a week, once or twice a month, and the like. In some embodiments, the amount, forms, and/or amounts of the different forms may be varied at different times of administration.

Dosing is dependent on severity and responsiveness of the disease state to be treated, with the course of treatment lasting from several days to several months, or until a cure is effected or a diminution of the disease state is achieved (e.g. reduction in tumor size). Optimal dosing schedules can be calculated from measurements of drug accumulation in the body of the patient. The administering physician can easily determine optimum dosages, dosing methodologies and repetition rates. Optimum dosages can vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and can be given once or more daily, weekly, monthly or yearly. The treating physician can estimate repetition rates for dosing based on measured residence times and concentrations of the drug in bodily fluids or tissues. Following successful treatment, it can be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

A person of skill in the art would be able to monitor in a patient the effect of administration of a particular agent. Other techniques would be apparent to one of skill in the art.

### EXAMPLES

### Example 1

### Generation of Stage-Specific Cancer Stem Cell Lines Via Mutagenesis

Low passage normal human ESC (hESC) or their neural stem cell derivatives (hNSC) are seeded under serum- and feeder-free conditions on extracellular matrix-coated cell culture dishes in the presence of mutagen such as ICR191, hydroxylurea, benzo(a)pyrene; (Klemm et. al, Toxicol In Vitro. 15: 447-53 2001; Scholz et. al, Cells Tissues Organs 165: 203-11, 1999). A large quantity of the same passage cells is set aside and frozen in multiple vials. These cells will constitute the unaltered hESC control cell population for the model. Cells exposed to mutagen are cultured at the density, which allows for observance and selection of individual surviving clones.

Emerging stem cell clones are transferred into separate wells, expanded and selected for acquired features of the transformed phenotype using multiple screens as described bellow. Positive stem cell clones, i.e. clones that exhibit one or more characteristics of malignant transformation, are further expanded and frozen. These stem cell clones represent seed cells for the stage-specific cell lines that are subsequently incorporated into a multi-stage stem cell carcinogenesis platform. In the absence of positive stem cell clones in initial screens, stem cell clones that have not acquired a transformed phenotype are subjected to further exposure to the mutagen(s) and periodically screened for tumorigenic and malignant phenotypes until such phenotypes emerge. A fraction of cells from the positive stem cell clones will be further, exposed to mutagenesis and selection process using additional screens to obtain cancer stem cell lines of more advanced stage along the malignant transformation continuum. Additional set of screens described below ensure selection of cells that exhibit more aggressive malignant phenotypes. Clones of more aggressive cancer stem cells are isolated, expanded, and frozen for incorporation into the multi-stage stem cell carcinogenesis platform.

### Example 2

### Phenotypic Screens for Tumorigenic and Malignant Phenotypes

Mutated hESC and hNSC clones that exhibit a transformed phenotype are isolated and expanded, and subsequently subjected to multiple screens that select for the cells that have acquired certain pre-malignant, tumorigenic or malignant characteristics. The following screens will be used:

Cellular Growth Upon Exposure to Mutagen(s)

Cell clones are initially selected for their ability to survive and proliferate in the presence of mutagen(s). Once selected clones are expanded, cell proliferation in the presence of mutagen(s) is assessed by bromodeoxyuridine (5-bromo-2-deoxyuridine, BrdU) incorporation as a secondary screen.

Loss of Spontaneous Differentiation

Mutated hESC clones are screened for the ability to maintain pluripotency as determined by alkaline phosphatase (AP) activity after removal of basic fibroblast growth factor (bFGF) from the cell culture medium. Unaltered hESC serves as a negative control for the mutated hESC clones. Secondary screen measuring the expression of pluripotency marker(s), including the key transcription factors that regulate ES cells, Oct 3/4 and/or Nanog, is used to confirm that the selected hESC clones maintain a pluripotent state under conditions that induce differentiation of the control hESC.

Loss of Ability to Differentiate Along a Specific Differentiation Pathway

hESC clones are screened for the ability to maintain a pluripotent state as determined by alkaline phosphatase (AP) activity after addition of cell culture medium containing a differentiation-inducing stimulus. For example, mutated hESC clones are exposed to retinoic acid that induces differentiation of hESC into neural lineages (Baharvand H, Stem Cells Dev 2008; Couillard-Despres et. al., BMC Neurosci 9:31, 2008 Erceg et. al., PLoS ONE. 3:e2122, 2008; McCaffery et. al. Eur J Neurosci 18: 457-72, 2003; McGuckin et. al. Nat Protoc 31046-55, 2008; Pierret et. al, Stem Cells Dev. 16:1017-26, 2007). Normal/unaltered hESC clones serve as a negative control. Secondary screen measuring expression of pluripotency marker(s), including transcription factors Oct 3/4 and/or Nanog, is used to confirm that the selected clones maintain a pluripotent state under conditions that induce differentiation of the control hESC clones. Mutated hNSC clones are exposed to neural stem cell differentiation mix (Daadi et. al, PLoS ONE 3:e1644, 2008) and screened for the absence of expression of the differentiation markers, i.e. for neurons: β-tubulin class III, microtubule-associated protein (MAP-2), neural cell adhesion molecule (NCAM), medium-size neurofilament (NF-M) or neuron specific beta III Tubulin (Tuj1); for astrocytes: glial fibrillary acidic protein (GFAP); and for oligodendrocytes: myelin basic protein (MBP) or galactocerebroside (GC); and/or lack of changes in cell morphology. Mutated hESC clones are exposed to differentiation conditions that promote generation of hNSC and screened for the lack of morphological change and for maintenance of pluripotency markers including AP and/or Oct 3/4 and/or Nanog.

Increased Ability to Grow in Soft Agar-Methyl Cellulose

Mutated hESC/hNSC clones are expanded and subsequently selected for their ability to form colonies in soft agar using a methylcellulose monolayer system (Biedermann and Landolph, Cancer Res. 47:3815-23, 1987; Eker and Sanner, Eur J Cancer Clin Oncol. 22:671-6, 1986; Lichtenberg et. al. Toxicol Lett. 75 193-3, 1995; Nagasawa et. al. J Invest Dermatol 88 149-53, 1987; Tsao et. al. Cancer Res. 45: 5139-44, 1985).

Changes in Cell Polarization and Cell Adhesion

Changes in mutated hESC and hNSC are measured using epithelial-mesenchymal transition (EMT). Individual epithelial cells are aligned abutting each other in a uniform array. Regularly spaced cell-cell junctions and adhesions between neighboring epithelial cells hold them tightly together and inhibit the movement of individual cells away from the epithelial monolayer. Epithelial cells are polarized. Mesenchymal cells, in contrast, generally exhibit neither regimented structure nor tight intracellular adhesion. Mesenchymal cells form structures that are irregular in shape and not uniform in composition or density. Adhesions between mesenchymal cells are less strong than in their epithelial counterparts, allowing for increased migratory capacity. Moreover, mesenchymal migration is mechanistically different from epithelial movement. Turning an epithelial cell into a mesenchymal cell requires alterations in morphology, cellular architecture, adhesion, and migration capacity. Commonly used molecular markers for EMT include increased expression of N-cadherin and vimentin, nuclear localization of β-catenin, and increased production of the transcription factors such as Snail1 (Snail), Snail2 (Slug), Twist, EF1/ZEB1, SIP1/ZEB2, and/or E47 that inhibit E-cadherin production. Phenotypic markers for an EMT include an increased capacity for migration and three-dimensional invasion, as well as resistance to anoikis/apoptosis.

Loss of cell polarization characteristic of epithelial cells is measured in hESC clones using α₆ integrin expression as a marker of the polarized state (Sumi et. al. Oncogene 26: 5564-76, 2007). Expression of vimentin and/or ability to migrate on collagen and gelatin are used as a secondary screen.

Invasiveness Through Basement Membrane

Mutated hESC and hNSC clones are subjected to in vitro invasion assay, which is well known in the art, to select the clones that show ability to invade through the basement membrane (Genbacev et. al. J Clin Invest. 97:540-50, 1996; Genbacev et. al. Dev Biol. 233: 526-36, 2001; Ilic et. al. Am J Pathol 159: 93-108, 2001; Janatpour et. al. Development 127: 549-58, 2000 McCaffery et. al. Eur J Neurosci, 18:457-72, 2003). Secretion of matrix metalloproteinases (MMPs) is used as a secondary screen.

### Example 3

### Characterization of Isolated Clones at Various Stages of Malignant Transformation

hECS and hNSC clones are screened for their tumorigenic and malignant properties. These clones are then isolated and expanded in vitro to obtain multiple freezes of the same passage. Extensive characterization on these clones is subsequently performed. The characterization includes but is not limited to evaluation of the expression of standard pluripotency and differentiation markers, such as Oct 3/4, Nanog, SSEA-4 for hESC; nestin, radial glial cell marker, 3CB2, vimentin for hNSC, expression of telomerase activity, proliferation capacity (BrdU incorporation), cell population doubling time, etc. To evaluate the level of genomic stability of the obtained stem cell clones, each stem cell clone is evaluated by karyotyping (G-banding) at the time of freezing and after 5 and 10 passages.

Transcriptional profile of the obtained stem cell clones is obtained using chromatin immunoprecipitation (ChIP) sequencing transcription assay. Since all of the stem cell chines are derived from the same hESC originally, all stem cell clones share the same genetic background, thereby reducing the level of noise attributed to heterogeneity of cells arising from different individuals within the human population. This will allow an easier identification of the molecules activated through mutagenesis that have contributed to acquirement of certain malignant traits such as invasiveness.

To identify chromosomal changes associated with the transformation, array comparative genomic hybridization (aCGH) is performed between isolated mutated hESC and hNSC and their unaltered stem cell counterparts. Gene identification by NMD inhibition (GINI) analysis is performed to identify the specific mutations that have been mutated within each stem cell clone. Pattern analysis of multiple mutated stem cell clones that have been obtained under specific screen allows for determination of targets and pathways that have been repeatedly affected and are therefore, likely causal for acquirement of certain phenotypic characteristics. Epigenetic changes that may have occurred during selection and screening process are assessed by DNA methylation analysis. Changes in microRNA regulation are also assessed by miRNA arrays. Samples from mutated hESC and hNSC are compared to the respective unaltered stem cell controls.

Using one hESC line, a panel of isogenic cloned cell lines has already been generated and banked. These cell lines stably exhibit transformed phenotypes associated with CSC including but not limited to formation of tumorospheres in vitro, generation of serially transplantable tumors in vivo at limited dilutions and increased resistance to common chemotherapeutic agents such as paclitaxel, doxorubicin, temozolomide and carmustin. High resolution karyotype (G-banding), proliferation rate (BrdU incorporation and doubling time), growth factor dependence and expression of CSC/SC markers such as Oct-4, Nanog, CD133, ABCG2, ABCG5 etc) have been examined and the stability of the expressed phenotypes was confirmed.

### Example 4

### Generation of the MSCC System/Platform

A spectrum of cancer stem cell lines from early i.e. less aggressive to late i.e. highly aggressive stages of carcinogenesis is produced using the methods described above. This approach allows comparison of generated cancer stem cells with normal i.e. unaltered stem cells within the same genetic background and at different levels of differentiation. Profiling data obtained on cell characterization and pattern analysis on stem cell clones of various stages of the stem cell malignant transformation is used to create a database and a predictive model of stem cell malignant transformation including information on the major molecules and genetic pathways involved in stem cell transitions within this continuum. This model provides for virtually unlimited source of homogeneous cancer stem cell population and its normal stem cell counterpart enabling a high-throughput screening as well as robust and highly reproducible results. Due to the nature of random mutation of this mutagenesis approach, the herein described multi-stage stem cell carcinogenesis system provides a nonbiased screen for mechanisms that affect malignant transformation of stem cells.

Validation

During the validation process, known inhibitors of a genetic pathway and/or molecule that have been altered or mutated in this mutagenesis process are used to demonstrate that inhibiting these pathways and/or molecules affects cellular properties including but not limited to cell proliferation, cell survival, cell differentiation, cell adhesion, cell migration, and invasiveness of the generated cancer stem cell lines. Chemotherapeutic reagents, such as rapamycin, known to preferentially affect cancer stem cells relative to normal untransformed stem cells are used to confirm that the same preferential effect is observed in this system. Chemotherapeutic reagents known to have low potency towards cancer stem cells are also used to demonstrate that these reagents have less effect on the malignant stem cell lines generated in this system than on commonly used cancer cell lines. The abovementioned validation tests and the obtained data therefrom are used to improve the predictive value of the multi-stage stem cell carcinogenesis system.

### Example 5

### Screening for Compounds that Preferentially Affect Cancer Stem Cells Over Normal Stem Cells

A small molecule library is screened for molecules that induce differentiation, quiescence, senescence, and apoptosis with a higher efficacy in pre-malignant and/or malignant stem cell lines than in normal i.e. untransformed hESC or hNSC or differentiated cells, i.e. neurons and glial cells derived from hNSC. The screening is performed in the presence and/or absence of other standard chemotherapeutic reagent such as DNA-damaging chemical (i.e. cisplatin, carboplatin etc.).

Derived early i.e. less aggressive and late i.e. highly aggressive cancer stem cell lines that are produced using the methods described above are seeded into 96-well or 384-well plates under serum-free and feeder-free conditions in the presence of the small molecule library. To determine small molecules that preferentially affect cancer stem cells relative to untransformed hESC, additional plates containing original hESC from which cancer stem cell lines were derived are exposed to the same library. Following exposure to the small molecule library, wells are processed for the cell number (i.e. CyQUANT® NF Cell Proliferation Assay Kit from Invitrogen) to determine cell proliferation/quiesence and survival/cell death. Parallel wells are processed for alkaline phosphatase activity (i.e. fluorimetric alkaline phosphatase assay from ANASPEC Sensolyte FDP) to determine loss of pluripotency/induction of differentiation. Molecules that inhibited proliferation/survival and/or induced higher level of differentiation in cancer stem cells relative to the parent hESC are selected for the secondary screen. Inhibition of cell proliferation/survival is confirmed by BrdU incorporation (i.e. ELISA-based BrdU assay with chemiluminescent detection from Roche). Loss of pluripotency/induction of differentiation is confirmed using ELISA-based assay for the expression of pluripotency markers Oct 3/4 and/or Nanog. To determine small molecules that preferentially affect cancer stem cells relative to untransformed hNSC from which they were derived and/or differentiated cells, i.e. neurons and glial cells derived from hNSC, plates containing original hNSC from which cancer stem cell lines were derived and/or their differentiated derivatives obtained by one of the standard differentiation protocols (Cai and Grabel, Developmental Dynamics 232:3255-3266, 2007) are exposed to the same library in the presence of the appropriate medium to support the cells. Following exposure to the small molecule library, wells are processed for the cell number (i.e. CyQUANT® NF Cell Proliferation Assay Kit from Invitrogen) to determine cell proliferation/quiesence and survival/cell death. In case of comparison to hNSC, parallel wells are processed for the ELISA-based assay for the expression of multipotent mitotic neural stem cell markers i.e. nestin, radial glial protein, vimentin, Sox2, Pax6, prominin/CD133, XCR4/CD184, integrin 131/CD29, SSEA-1/LEX/CD1, TAPA1/CD81, CD34, CD45, CD24, Musashi1, GFAP and/or BLBP. In the case of comparison to differentiated neural or glial cells, parallel wells are processed for the ELISA-based assay for the expression of terminal differentiation markers i.e. calretinin, neuron-specific enolase, NeuroD1, HuC/D, NeuN, Tuj-1, calbindin, Tau, neurofilament, MAP2ab, tyrosine hydroxylase, NF145, VIPO, choline acetyltransferase, and/or tryptiphan hydroxylase. Molecules that inhibited proliferation/survival relative to the parent hNSC and/or differentiated cells derived from hNSC and/or induced higher level of differentiation in cancer stem cells relative to the parent hNSC are selected for the secondary screen. Inhibition of cell proliferation/survival is confirmed by BrdU incorporation (i.e. ELISA-based BrdU assay with chemiluminescent detection from Roche) and secondary differentiation screen is performed using ELISA-based assay for the expression of additional marker of hNSC i.e. RIP, O1, O4, CNPase, PSA-NCAM, DCX, GalC, NG2, S100β and/or A2B5 (Eric Wexler: Markers of Adult Neuronal Stem Cells in Methods in Molecular Biology, vol. 438: Neural Stem Cells, Edited by: L. P. Weiner© Humana Press, Totowa, N.J.).

A pilot screen has been performed using four of the lines from the cell panel as disclosed herein (i.e. control hESC and 3 transformed lines) and 50 selected compounds. It has been shown that small molecules can be identified that differentially affect normal hESC and the generated CSC. Without being bound by any theory, from known mechanisms of action of these compounds, some of the key regulatory pathways that are altered in the transformed lines relative to the normal controls can be inferred.

### Example 6

### Conversion of Human Embryonic Stem Cells into Neural Stem Cells (NSC) in Defined Adherent Cultures hESC Culture and Neural Conversion

Growing colonies were disaggregated with Accutase, cell detachment agent that does not contain any mammalian or bacterial-derived proteins (Millipore). Disaggregated cells were washed once in MEM and seeded onto CellStart-coated dishes in StemPro NSC SFM (Invitrogen). A fraction of cells did not attach and continued to grow in suspension forming neurospheres, whereas attached cells formed a monolayer and were continuously expanded under adherent conditions. For neural differentiation, the cells were seeded onto poly-L-ornithine/laminin-coated dishes and cultured in StemPro NSC SFM without bFGF for six weeks.

Immunostaining

Cells were fixed in either 90% acetone for 10 minutes or in 3.8% paraformaldehyde, pH 7.4-7.6, and then permeabilized with 90% acetone or 0.2% Tween-20 for 10 minutes. Samples were incubated with the following primary antibodies: anti-nestin, -β1 integrin, -neuron specific nuclear protein, NeuN (Chemicon), -α6 integrin, GoH3, -E-cadherin (BD Biosciences), tubulin (Covance), -myelin basic protein, MBP, -glial fibrillary acidic protein, GFAP (Imgenex), and radial glial protein 3CB2 (Developmental Studies Hybridoma Bank at University of Iowa). All secondary antibodies were purchased from Jackson ImmunoResearch, whereas Hoechst 33342 was obtained from Invitrogen.

Epithelial-Like Polarity is Associated with hESC Pluripotency

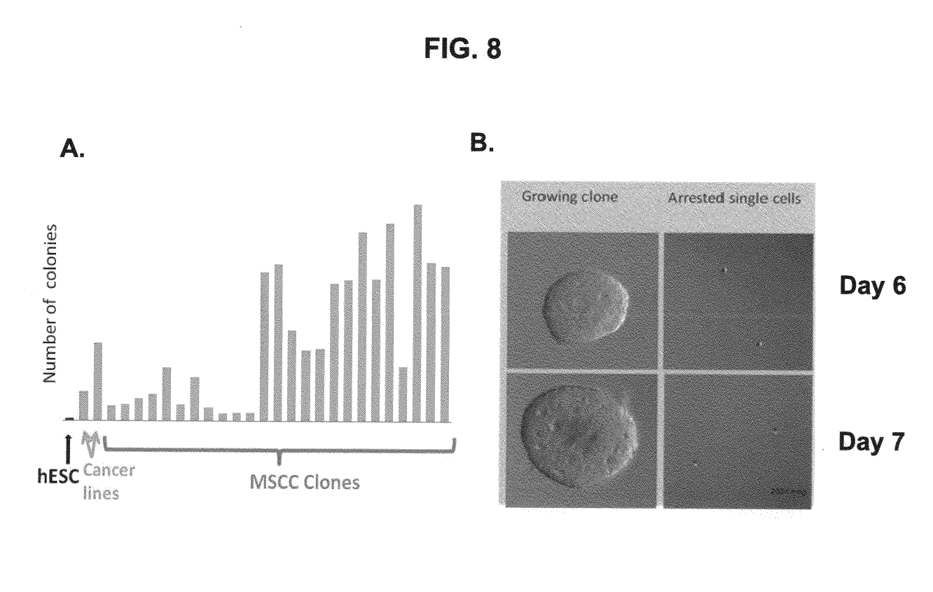

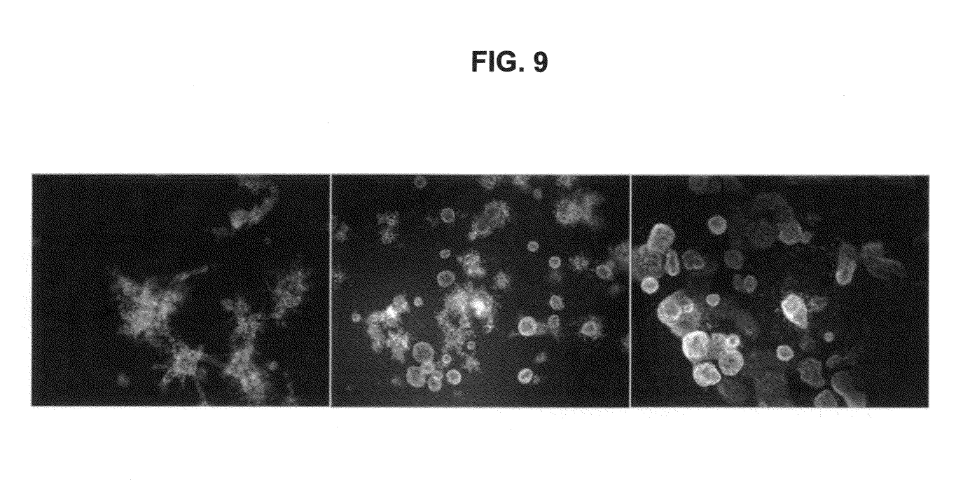

Depriving cells of cell-cell contact signals and therefore, apical-basal polarity, is important for neural commitment in two very different systems: Xenopus ectoderm and mouse ESC [2,40,41,45-47]. In the human system, undifferentiated hESC exhibit epithelial-like apical-basal polarity, and its disruption in hESC enhances hematoendothelial differentiation [47]. To determine whether polarity is correlated with hESC pluripotency regardless of culture conditions, expression of integrin subunit a6 was examined at the initial stages of hESC derivation as well as in hESC culture on defined human ECM in defined growth factor-rich medium [48,49]. Seventy two hours after transferring of the cleavage-stage biopsied blastomere onto HFF feeder layer, expression of a6 integrin subunit was already detected in the initial outgrowth (FIG. 8). Established hESC lines can be grown not only on feeder cell layers but also on feeder-free ECM substrate without loss in expression of pluripotency markers (data not shown) or obvious alteration in compact appearance of hESC colonies (FIG. 9a, b). Since cell-ECM interactions may modulate expression of proteins involved in both cell-ECM and cell-cell adhesion processes and maintenance of polarity, it was examined whether expression of a6 integrin was preserved in hESC colonies grown on a defined human ECM. Indeed, expression of a6 integrin remained as high as in hESC colonies seeded on feeders or on a Matrigel (FIG. 9c-e; data not shown).

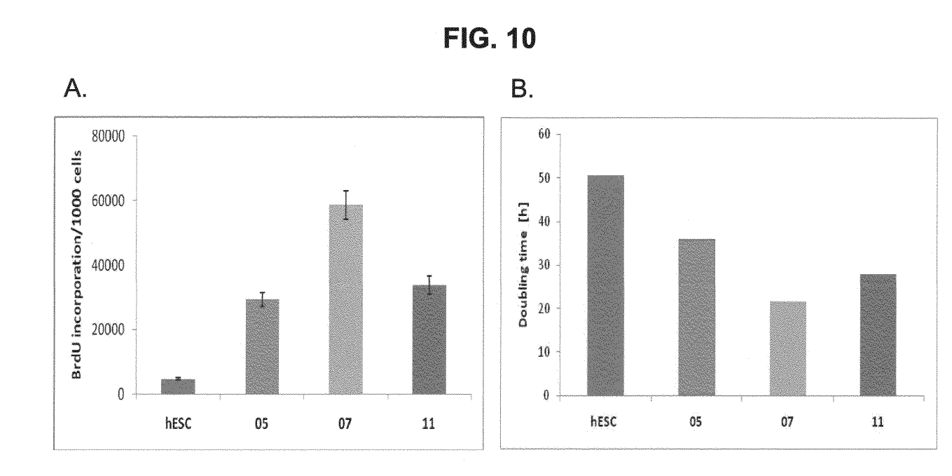

Taken together, the results suggest that the presence of cell-cell contacts is associated with pluripotent hESC regardless of the substrate on which the cells were grown. Therefore, disrupting of cell-cell contacts may be required to induce neural conversion of hESC. However, disrupting polarity and cell-cell signaling alone is not sufficient to induce neural conversion of hESC in the presence of self-renewing signals (i.e. bFGF and activin) [49]. Generally, within a couple of days, a single-cell suspension of hESC seeded on CellStart in pluripotency supporting serum-free medium generates the typical colonies of polarized hESC (FIG. 10).

Disaggregated hESC Seeded on Defined Human ECM in Growth Factor-Poor Medium Supplemented with bFGF Convert into NSC

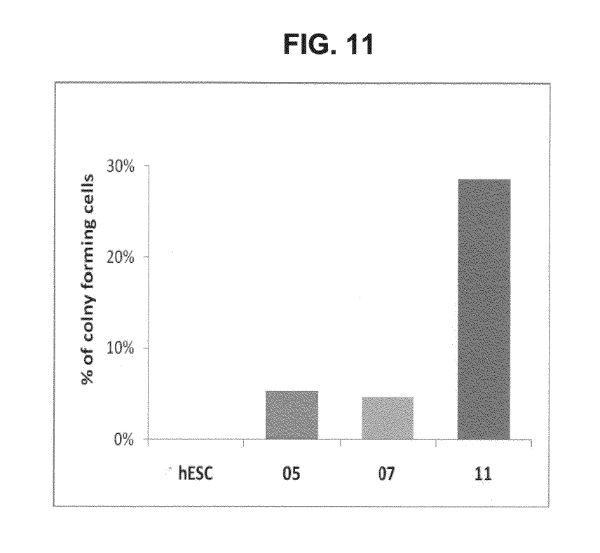

The next set of the experiments tested whether default neural conversion applies to hESC. The approach was first to disrupt polarity and deprive hESC cells of signals from cell-cell contacts by making single-cell suspension, and then seed them onto a defined human ECM in growth factor-poor medium. hESC colonies were disaggregated with accutase, a mixture of proteolytic and collagenolytic enzymes that does not contain products of mammalian or bacterial origin. Cells in suspension were then seeded into CellStart-coated dishes in StemPro NSC SFM medium. The medium was supplemented with bFGF, since it has been reported that bFGF triggers transition of pluripotent ESC from self-renewal to lineage commitment in both mouse and human ESC [5,42]. A fraction of cells did not attach and continued to grow in suspension forming neurospheres, whereas attached cells continued to grow as adherent cultures. Upon reaching 60-80% confluency, cells in adeherent cultures were dissociated with accutase, re-plated under the same conditions and cultured for several passages. Rare typical hESC colonies were observed to be scattered among the attached cells in the first passage. These colonies disappeared after the second passaging, and all cells were uniformly distributed in a monolayer (FIG. 11).

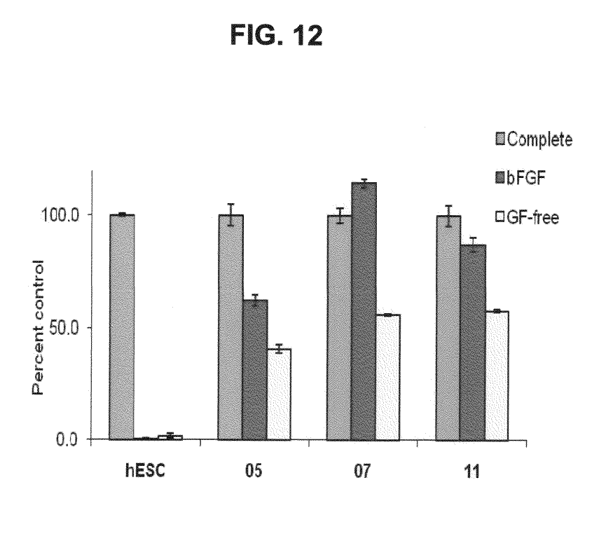

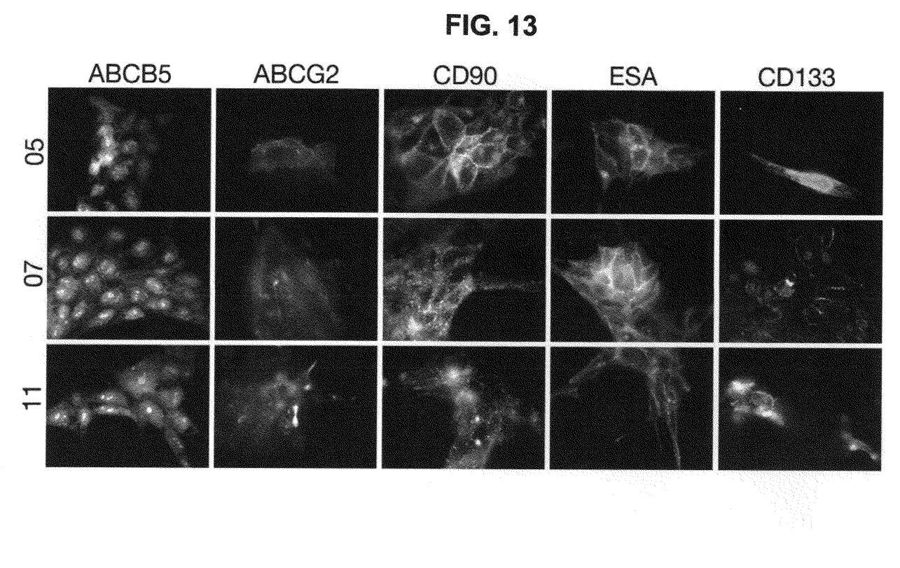

To assess whether cells underwent neural conversion, cells grown in adherent cultures were examined three days later for the presence of neural markers by immunocytochemical analysis (FIG. 12). All cells expressed radial glial protein and nestin, markers of the neural lineage, and they also expressed Sox-1, a marker of neural stem cells, indicating that this population represents one of the first steps in the conversion of hESC into the neural lineage. To demonstrate that these cells were indeed NSC and were thus capable of differentiating into the main cell-types of the central nervous system, cells were seeded into poly-L-ornithine/laminin-coated dishes and left to differentiate in StemPro NSC SFM without bFGF for six weeks. Immunocytochemical analysis revealed the multipotent nature of the obtained NSC, which were able to differentiate into the three principle cell-types of the central nervous system: neurons, astrocytes and oligodendrocytes (FIG. 13). Therefore, the present invention provides a method for directly converting hESC into NSC under fully defined xeno-free adherent culture conditions, thereby avoiding heterogeneity of cell phenotypes that form within the neurospheres.

Conclusion

In one embodiment, the present invention presents an attractive method of hESC neural induction. The major advantages of this method include but are not limited to: 1) it is simple and reproducible, 2) it does not require neurospheres, 3) the resulting neurally committed cells are highly pure and uniform population, 4) the conversion is achieved under defined xeno-free conditions, and 5) the homogenious population of neurally committed cells can be continuously expanded under adherent conditions. Defined conditions under which the method of the present invention is developed would help not only to identify the molecular nature of neural induction in hESC, but also serve as a starting point for further improvements of in vitro differentiation protocols used in the development of stem cell therapies and drug screening for multiple neural disorders.

### Example 7

### Generation and Screening of Over 20 Transformed Cell Lines at Varying Stages of Carcinogenesis

Passage 20 hESC grown under controlled and defined conditions were subjected to mutagenesis in the presence of frame-shift mutagen, ICR 191 for up to 6 months. After initial 15 weeks of exposure a portion of cells was removed and single cells were seeded to obtain clones. Clonogenic efficiency was 15%. Obtained individual twenty-five clones proliferated in culture with varying rates. All clones were expanded and banked.

To select the cell lines with characteristics of cancer cells, clones were screened for proliferation, non-adherent growth, invasive phenotype and their response to commonly used chemotherapeutic reagents (anti-proliferative cancer drugs). Obtained results confirmed prediction of MSCC model that can be generated through chemical mutagenesis multiple cell clones with different growth and drug resistance profiles.

Additional clones were generated by in vivo selection from the tumors formed by cells exposed to mutagenesis for 3 or 6 months and subset of in vitro-isolated clones. Preliminary phenotypic analysis suggests that we have obtained both clones with cancer stem characteristics and those with more differentiated characteristics of cancer cells that likely represent the progeny of cancer stem cells.

Non-Adherent Growth/Tumorsphere Formation

Cells' capacity for non-adherent/anchorage-independent growth is one of the common characteristics of cancer cells and often correlates with their tumorigenicity (Kahn and Shin, JCB, 1979). Formation of tumorspheres in vitro is a property of CSC-enriched cancer cell populations (Gupta et al., Cell 138, 2009). To examine the ability of generated clones for anchorage-independent growth/tumor sphere formation, their growth was tested in methyl cellulose assay (FIG. 8) using previously described protocol with minor modifications (Kahn and Shin, JCB, 1979). Clone colony formation ability in methyl cellulose was compared to that of the control hESC and two established cancer cell lines, PAI-1 and NCCIT. All 25 clones examined grew under non-adherent conditions while control hESC did not. Among these, ten clones (40%) showed a significantly higher number of colonies than either of examined cancer lines, whereas other clones exhibited similar or lower anchorage-independent growth capacity (FIG. 8). Among 25 clones, 4 exhibited slow rate of proliferation in adherent cultures and were eliminated. The remaining 21 clones were used for further screening.

Expression of Invasive Phenotype

Ability to invade through a basement membrane is associated with the malignant state. To determine which clones have acquired an invasive phenotype, single cells were re-suspended in the basement membrane extract (Matrigel) and examined for their ability to penetrate into matrix and/or proliferate. Out of 21 clones tested, 6 (28.5%) exhibited highly invasive phenotype, 6 (28.5%) a moderately invasive phenotype and 9 (43%) showed little or no invasiveness (FIG. 9). These results provide further evidence that cloned lines acquired some of the key characteristics of cancer cells and that they exhibit various degrees of aggressiveness.

Drug Resistance

Cancer stem cells often exhibit a high level of resistance to common chemotherapy. To select clones for further evaluation, viability screening was performed in the presence of five common chemotherapeutic reagents.

Resistance to Carboplatin

First, resistance to alkylating agent carboplatin commonly used in the treatment of ovarian, lung, head and neck cancers was tested. The initial cell viability data showed that clones varied in their resistance to carboplatin (data not shown). Seven clones that were previously determined to be low or non-invasive and that exhibited very low level of resistance to carboplatin were eliminated from further screening.

Resistance to Irinotecan, Mitomycin C, Taxol, and 5-fluorouracil

Next, survival of the remaining 14 clones was tested in the presence of irinotecan, mitomycin C, taxol, and 5-fluorouracil. As expected, the results indicated that tested clones varied in their drug resistance profile.

Conclusion

Over 20 isogenic cell lines have been generated that exhibit varying degrees of the measured transformed phenotypes including anchorage-independent growth, invasiveness and resistance to chemotherapeutic reagents. The preliminary analysis of in vivo selected clones indicates that both cells with cancer stem cell characteristics and those with more differentiated cancer cell phenotype have been obtained. Taken together, isogenic lines at different stages of carcinogenesis have been generated.

### Example 8

### Initial Characterization of Selected Cell Lines and Validation of Key Cancer-Like Characteristics

Based on the results from our initial screening, we have chosen three clones with high resistance to one or more drugs that were invasive in Matrigel assay and had the ability for anchorage-independent growth and formation of tumorspheres. These three clones (05, 07, and 11) were subjected to further characterization.

Karyotype

To examine the level of chromosomal abnormalities in the mutated clones, these clones were subjected to karyotyping with G-banding. The results are presented in the Table 1.

TABLE 1
Karyotype analysis of the original hESC and selected mutated clones

| hESC | 05 | 07 | 11 |
|---|---|---|---|
| 46, XX | mos 46, XX, | 46, XX, | 46, XX |
| (100%) | add(20)(q11.2) | del(11)t(1; 11)(q25; q23) (100%) | (100%) |
| | (70% abnorm.) | | |

While the original hESC line has normal female karyotype, 05 and 07 clones showed limited number of chromosomal abnormalities and clone 11 did not exhibit any gross chromosomal changes. Repeated karyotyping of the parental hESC and clones 07 and 11 revealed stability of their karyotypes after >20 passages in culture. Repeated karyotyping of the clone 05 is in progress.

Proliferation Rate

To examine the proliferation rate of the selected clones, BrdU incorporation of the exponentially growing cells was measured after 3 days in culture (FIG. 10A). Results from nine independent experiments performed using cells at different passages show that all of the selected clones have a significantly higher proliferation rate than the original hESC. Among all clones tested, clone 07 has the highest proliferation rate. The same trend was confirmed by measurement of cell population doubling time during 7-day culture (FIG. 10B).

The more dramatic difference in growth rate observed by BrdU incorporation as compared to the doubling time measurement is due to the fact that 3-day BrdU measurement does not take into account significant increase in hESC growth rate once they reach more than 50% cell density, typically on day 3 or 4 of the culture. This effect most likely stems from hESC dependence on self-produced extracellular matrix and autocrine factors that become more available as the density of cultures increases.

Anchorage-Independent Growth

Formation of tumorspheres is associated with cancer stem cell phenotype. To confirm that selected clones retained their ability for anchorage independent growth, formation of tumorspheres in methyl-cellulose at different passages has been tested. The results show that control hESC did not form tumorspheres in methyl cellulose even after 20 days of culture while all three clones exhibited to various degrees the ability for anchorage-independent growth (FIG. 11). Differences between cell lines in tumorsphere-forming capacity remained stable with cell passaging.

Growth Factor Independence

One of the key characteristics of transformed cells is their ability for uncontrolled growth and thus, decreased dependence on the presence of the external growth factors for their survival and proliferation. Indeed, growth factor-independence is one of the hallmarks of aggressive tumors. To evaluate whether selected clones exhibited growth factor independence, these clones were cultured in the growth factor-free medium or in the medium containing only bFGF and evaluated their proliferation rate by BrdU incorporation. Results summarized in FIG. 12 show that while normal hESC did not grow in the absence of the growth factors, all tested clones did, with 07 being the most resilient.

To evaluate the effect of growth factor depletion on long term proliferation of the clones and compare them with the parental hESC, these clones were cultured for multiple passages under each of described conditions. Since the parental hESC did not exhibit a significant growth beyond the initial passaging, these cultures were terminated. In accordance with BrdU data, clone 7 exhibited the highest growth-factor independence. While in the absence of growth-factors proliferation of the other two clones was limited beyond the first passage, 07 continued to grow, albeit with a slower growth rate, for more than 5 passages. In the presence of bFGF alone, both 07 and 05 retained long-term ability to proliferate (>5 passages), while clone 11 was not capable of significant growth after the first passaging (data now shown).

Selected clones were further characterized by immunophenotyping for stem and cancer stem cell markers. Results are summarized in Table 2 and FIG. 13. These results confirm that generated clones express cancer and stem cell (CSC/SC) markers. Two of the clones have acquired expression of c-Kit, a tyrosine kinase receptor whose overexpression is associated with increased survival signals in multiple cancer stem cell types.

TABLE 2

| CSC/SC marker | hESC | 05 | 07 | 11 |
|---|---|---|---|---|
| Oct4 | + | + | + | + |
| SSEA4 | + | +/− | + | + |
| CD34 | − | +/− | −/+ | − |
| CD44 | − | −/+ | +/− | − |
| CD90 | + | + | + | + |
| CD117 (c-Kit) | − | + | + | − |
| CD133 | + | +/− | + | − |
| CDw338 | + | + | + | + |
| (ABCG2) | | | | |
| ABCG5 | + | + | + | + |
| ABCB5 | + | + | + | + |
| ESA/EpCAM | + | + | + | + |

Table 2: All 3 clones express markers of stem and cancer stem cells.
+ indicates positive staining >80% of cells;
+/− indicates 10-50% positive cells;
−/+ indicates low level of positive staining in <50% of cells;
− indicates no positive staining (<1%).

Table 2: All 3 clones express markers of stem and cancer stem cells. + indicates positive staining >80% of cells; +/− indicates 10-50% positive cells; −/+ indicates low level of positive staining in <50% of cells; − indicates no positive staining (<1%).

Representative immunophenotyping results are shown in FIG. 12.

Conclusion

Isogenic cell lines have been generated using a mutagenesis strategy that allowed random accumulation of mutations and non-biased in vitro screens for the key characteristics of the sternness, premalignant and malignant states. Taken together, the results show that selected cell lines exhibit to varying degrees main cancer-like and cancer stem cell-like characteristics including invasiveness, expression of cancer stem cell markers, tumorsphere formation and growth factor independent-growth. Therefore, developed platform of the present invention provides a model that can recapitulate the molecular signatures of different stages of carcinogenesis and thus, enables detection of specific drug candidates and targets at each of these stages.

### Example 9

### Drug Screening for Resistant Phenotypes

Drug resistance profiles of selected clones were determined for the common classes of chemotherapeutic reagents. Proliferation rate of the clones and parental hESC was measured in the presence of multiple concentrations of 10 different drugs using BrdU incorporation assay. Each drug has been tested in at least three independent experiments, each condition being done in triplicate. The approximate half maximal inhibitory concentration (IC₅₀) was determined for each of the drugs tested. IC₅₀ represents the concentration of a drug that is required for 50% inhibition in vitro.

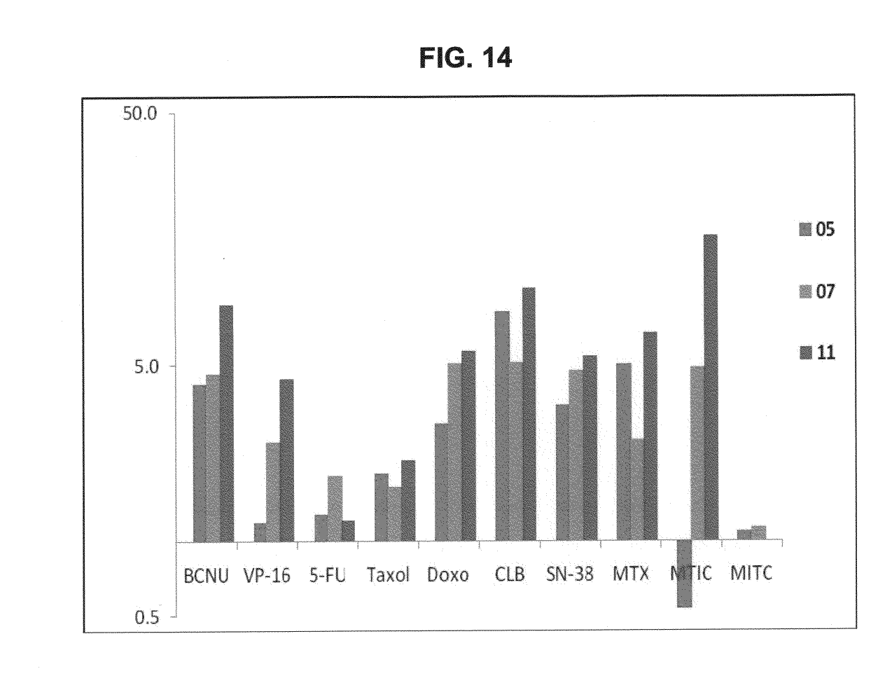

Response of the selected clones to the following chemotherapeutic reagents was examined. The chemotherapeutic agents were chosen for their wide-spread use and variety with respect to their modes of action: 5-fluorouracil (5-FU), doxorubicin (Doxo), irinotecan (SN-38), taxol, etoposide (VP-16), carmustine (BCNU), methotrexate (MTX), cytarabine (AraC), temozolomide (MTIC) and chlorambucil (CLB). Exposure to mitomycin C (MITC) was used as a positive control and induced growth arrest in all cell lines. Proliferation rate was measured by BrdU incorporation at the end of the drug treatment. All samples were made in triplicates in each of the three independent experiments performed. Results for the ten drugs are summarized in the FIG. 14.

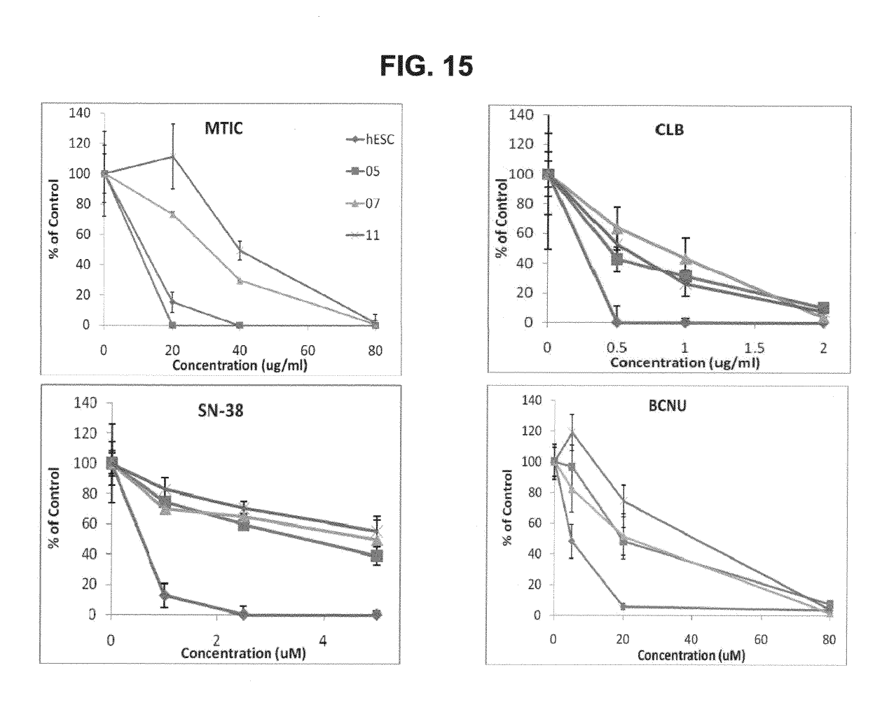

Examples of the dose response curves from the representative experiments with four of the drugs are shown in FIG. 15.

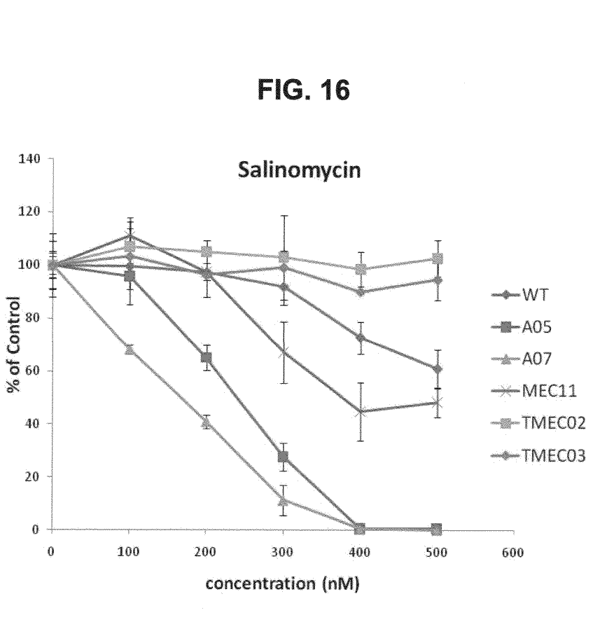

Resistance of various clones and isogenic cancer lines to CSC-targeting drug salinomycin is shown in FIG. 16. The tested lines exhibit varying resistance to salinomycin, a compound with selective cytotoxic effect towards epithelial CSC (Gupta et al., Cell, 2009). Proliferation of the 07 and 05 clones, was most significantly affected by salinomycin suggesting that they may have acquired a complete CSC phenotype.

Two clones, 07 and 11 were exposed to increasing concentrations of BCNU. The surviving cells were cloned and a number of high-resistance clones two of which, 07-B1 and 11-B19 were isolated. Their drug resistance to BCNU and MTIC was tested. The results indicate that both clones showed higher resistance than the parental clones to both BCNU and MTIC, indicating that the selection with a single drug conferred higher resistance to other drugs (data not shown).

Conclusion

Drug resistance tests show that examined clones exhibit higher resistance to a variety of common cytotoxic reagents than the control cells. The only exception is clone 05 which is more sensitive to MTIC than control cells. At the same time, cell lines differed in their drug resistance profiles. Whereas clone MEC-11 has been the most resistant to 5 out of 10 drugs tested, clone 07 was more resistant to 5-FU. Additionally, clones were generated with increased drug resistance by selecting growing clones during exposure to a single drug.

### Example 10

### Initial In Vivo Screening for Tumor Initiating Capacity

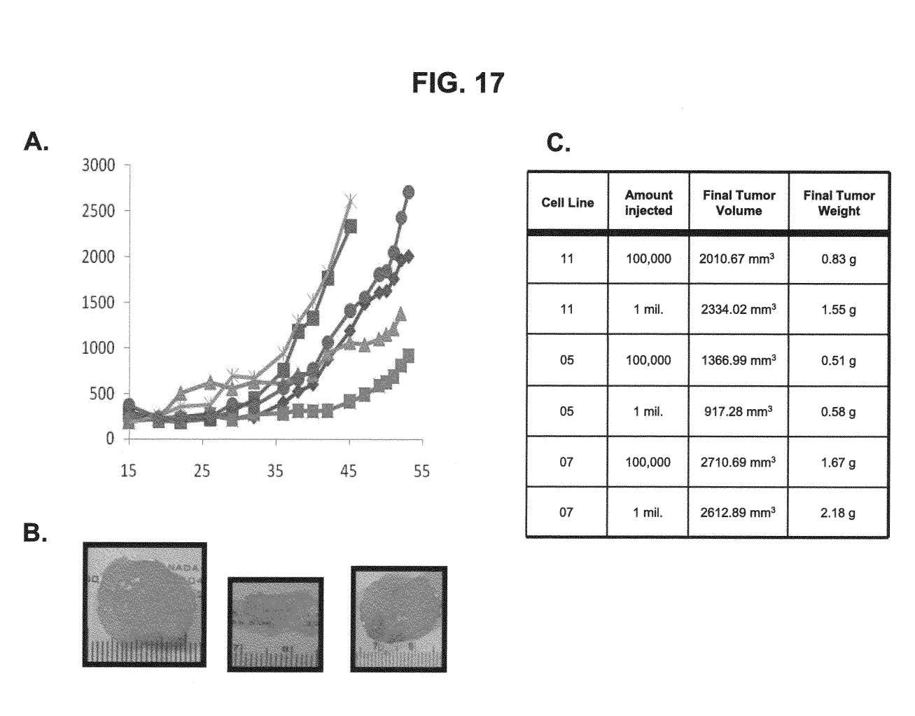

Tumor-initiating and cancer stem cells are characterized by their ability to form tumors from the small numbers of cells injected into experimental animals. To test their tumorigenicity, 100,000 and 1 million cells from the three clones (05, 07, and 11) were injected subcutaneously into immunocompromised mice. Tumor growth was monitored every two to three days (FIG. 17). Tumors were harvested when they reached volume of ˜2000 mm³ or at the end of experiment and used for serial transplantation and isolation of the cell lines.

As depicted in FIG. 17A, all of the cell lines formed tumors in immunocompromised animals independent of the number of cells injected. However, the kinetics of tumor formation was dependent on the number of injected cells with higher number of cells giving tumors 5-10 days faster than the lower number.

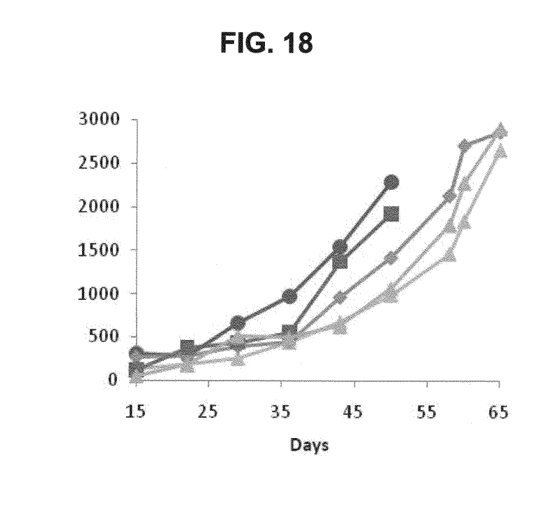

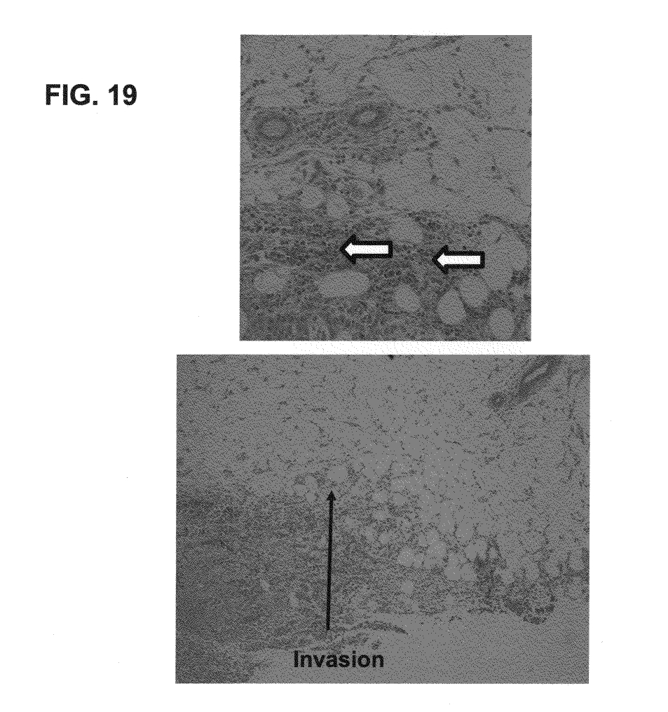

The ability of the clone 07 that was the most rapidly growing clone was further tested in vitro and in vivo, to form transplantable tumors, one of the key characteristics of CSC/TIC. 100,000 and 10,000 cells isolated from the initial tumor were re-injected into immunocompromised mice and measured their ability to form tumors (FIG. 18). The rate of tumor growth remained unaltered between primary and secondary tumors, suggesting a stable tumorigenic phenotype of 07 cell line. When tumor cells were isolated from 07 tumors and tumor stem cell population selected from differentiated progenitors, the immunocytochemical and morphological analyses revealed that they retained the expression of SC/CSC markers and reassumed the same morphology in culture as the injected population. Pathohistological analysis of the tumors revealed invasiveness into a host tissue, a clear sign of malignant nature of the tumor (FIG. 19), and tumor-formed irregularly dilated vascular channels indicating angiogenic activity associated with aggressive tumors and poor prognosis (FIG. 19).

Conclusion

Initial animal tumorigenicity studies show that selected clones can form tumors in the immunocompromised mice even when injected in low numbers, thus confirming their tumor-initiating capacity. Furthermore, most rapidly growing tumors formed by clone 07 are transplantable and capable of re-forming even when only 10,000 cells are re-injected. Preliminary data suggest that clone 07 expresses a stable tumorigenic phenotype. Pathohistological analysis of the 07 tumors revealed existence of the malignant cells that invaded surrounding host tissue and high tumor-mediated angiogenesis. This indicates that 07 exhibit highly aggressive tumor phenotype that is associated with limited drug delivery and poor patient prognosis.

### Example 11

### Differentiation into Neural Stem Cells and Transformed Neural Stem Cells

Neural stem cells (NSC)

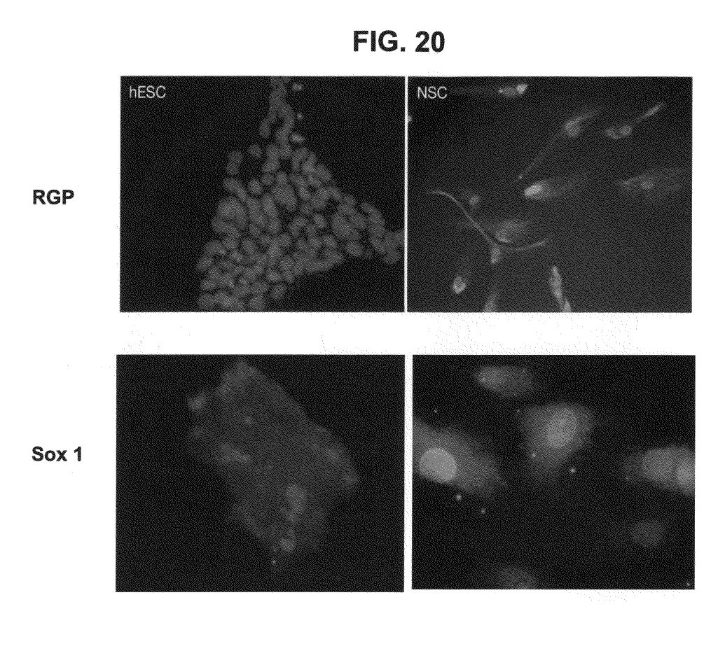

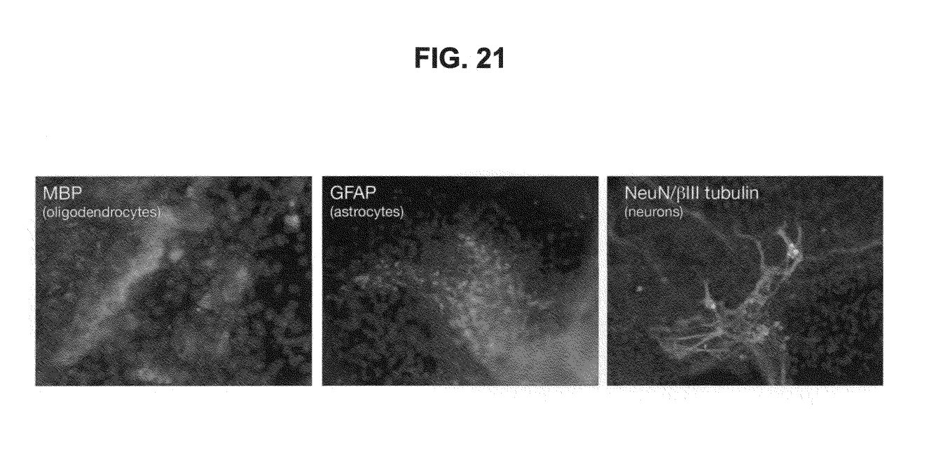

The strategy for differentiation of hESC line into NSC was to first disrupt polarity and deprive hESC cells of signals from cell-cell contacts by making single-cell suspension, and then to seed them on a defined human ECM CellStart in StemPro NSC medium. A fraction of cells did not attach and continued to grow in suspension forming neurospheres, whereas the attached cells continued to grow as adherent cultures but assumed more spindle-shaped morphology and ceased to form colonies. Upon reaching 60-80% confluency, cells in adherent cultures were dissociated with accutase, re-plated under same conditions and cultured for several passages. To assess whether cells underwent a neural conversion, cells grown in adherent cultures were examined by immunocytochemical analysis for the expression of markers of neural lineage (FIG. 20), and for their ability to differentiate into the three main cell types of central nervous system: neurons, astrocytes, and oligodendrocytes (FIG. 21).

As shown in FIGS. 20 and 21, hESC-derived NSC express markers of neural lineage and neural stem cells: radial glial protein and Sox 1, and are capable of giving rise to main cell types present in central nervous system confirming their multipotent potential.

Transformed Neural Stem Cells

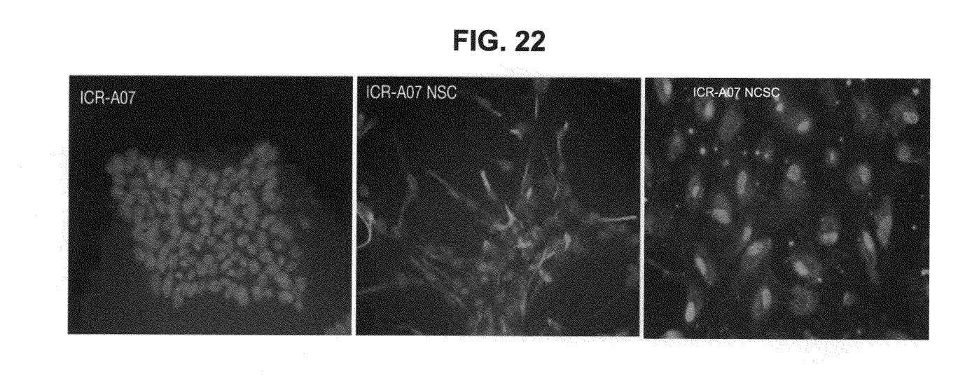

In order to generate transformed neural stem cells, three selected clones (05, 07, and 11) were subjected to identical culture conditions that yielded conversion of normal hESC into NSC. However, cells from only one of the clones, clone 07, assumed spindle-shape morphology and started to express NSC markers, glial radial protein and Sox 1 (FIG. 22). Unlike hESC-generated NSC, these cells still retained capacity to spontaneously form colonies in culture, indicative of less differentiated phenotype.

The capacity of these cells to differentiate into three main cell types of the central nervous system was then tested under conditions identical to those used for normal hESC. However, after 6 weeks of culturing, a glial fibrillar acidic protein (GFAP) could not be detected, indicative of astrocyte differentiation while the neuronal and oligodendrocyte markers were present. In addition, a significant fraction of cells (˜40-50%) retained expression of neural stem cell marker, Sox-1, suggesting lack of/aberrant differentiation in this population (data not shown).

Conclusion

Normal and mutated cell lines with characteristics of neural stem cells have been generated. In vitro specification data suggest that the neural stem cell-like line derived from a tumorigenic clone 07, exhibits the aberrant differentiation indicative of transformed phenotype.

### Example 12

### Generation of Animal Xenograft Models

In order to fully evaluate physiological effects and pharmacological properties of drug candidates, compounds selected during initial in vitro screening, will be further tested in animal models. Preliminary pathohistological analysis of serially transplantable tumors generated by one of the malignant lines indicates that the transplanted tumors retain substantially the same histological characteristics and growth rate as the original tumors and can therefore serve as a good xenograft model for animal studies. In some embodiments, additional xenograft models can be generated from the transformed lines derived from hESC lines with diverse genetic backgrounds.

The MSCC platform of the present invention enables creation of animal models from the same cell lines that were used for in vitro drug screening and thus, form tumors driven by/that arise from the same molecular characteristics/signature. This provides data consistency between cell-based assays and animal studies. Furthermore, MSCC animal models facilitate expression of the aspects of the tumor cell phenotype that are not expressed in culture such as the effects of angiogenesis and formation of the tumor vasculature and of tumor-stromal interactions on drug delivery.

Universal Animal Models

To generate xenograft models that will capture universal properties of CSC/TIC, immunocompromised mice will be injected with cloned cell lines that express stable CSC phenotypes including the ability to form serially transplantable tumors and use them for in vivo drug screening. If inhibition of tumor growth is observed without complete tumor eradication, efficiency of tested compounds in affecting CSC/TIC may be evaluated by transplantation of the remaining tumor mass into another animal. One such xenograft animal model using 07 line may have been generated that awaits validation.

To better monitor the ability of particular drugs to affect tumor growth, the original lines can be modified prior to injection by introduction of variety of reporter genes such as EGFP, luciferase, □ galactosidase etc. Effects of stromal interactions on tumor growth may be tested by co-injection of hESC-derived stromal cells together with CSC/TIC line.

Site/Tissue-Specific Animal Models

While common CSC/TIC properties stem from their adoption and alteration of developmental pathways that are shared with pluripotent stem cells, different cancer types and their CSC/TIC populations often exhibit a number of site and tissue-specific characteristics including expression of specific differentiation markers, amount of blood supply/hypoxia, response to growth factors and hormones etc. In order to generate site/tissue specific animal models, two general approaches will be used 1) in vitro generated transformed tissue-specific stem cells such as NCS generated from 07 line will be used and injected at the orthotopic site (e.g. brain for 07-NSC) or 2). Non-differentiated clones will be injected at various sites and monitored if they assume site-specific tumor growth and phenotype. In both cases, the presence of CSC/TIC will be monitored and assessed by serial transplantation. Additional sub-clones can be generated by isolating cells from the formed tumors.

In this site/tissue-specific animal model, monitoring of tumor growth may be enhanced by modifying the original lines using variety of reporter genes including but not limited to EGFP, luciferase, and galactosidase. Effects of stromal interactions on tumor growth may also be tested in site/tissue specific models by co-injection of tissue appropriate hESC-derived stromal cells together with CSC/TIC line.

### Example 13

### Three Dimensional Tumorsphere Screening Assay (3DT Assay)

Cell phenotype and behavior, including its drug resistance properties, are dependent on its 3D microenvironment. Therefore, in some embodiments, it may be beneficial to screen compounds using three dimensional cell culture system in addition to/instead of using standard 2D adherent cell culture system. Since variety of the generated clones from MSCC model can grow similar to tumorspheres in either liquid (i.e. media in low-adherent plates) or semi-solid (e.g. methyl cellulose, soft agar) cultures, they are well suited for this type of assays. Control cells may be generated from parental hESC line by inducing uniform EB formation in 96 well- or other multi-well plates using cell pellets of known sizes. Depending on the length of differentiation protocol and media components, control may be adjusted to contain higher or lower fraction of fully differentiated and progenitor cells thus enabling assessment of compound cytotoxicity on variety of isogenic non-transformed/normal cell types.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

[1] Munoz-Sanjuan, I. and Brivanlou, A. H. (2002). Neural induction, the default model and embryonic stem cells. Nat Rev Neurosci 3, 271-80.
[2] Ying, Q. L., Stavridis, M., Griffiths, D., Li, M. and Smith, A. (2003). Conversion of embryonic stem cells into neuroectodermal precursors in adherent monoculture. Nat Biotechnol 21, 183-6.
[3] Stern, C. D. (2005). Neural induction: old problem, new findings, yet more questions. Development 132, 2007-21.
[4] Dang, L. and Tropepe, V. (2006). Neural induction and neural stem cell development. Regen Med 1, 635-52.
[5] Kunath, T., Saba-El-Leil, M. K., Almousailleakh, M., Wray, J., Meloche, S. and Smith, A. (2007). FGF stimulation of the Erk1/2 signalling cascade triggers transition of pluripotent embryonic stem cells from self-renewal to lineage commitment. Development 134, 2895-902.
[6] Cai, C. and Grabel, L. (2007). Directing the differentiation of embryonic stem cells to neural stem cells. Dev Dyn 236, 3255-66.
[7] Martin, G. R., Wiley, L. M. and Damjanov, I. (1977). The development of cystic embryoid bodies in vitro from clonal teratocarcinoma stem cells. Dev Biol 61, 230-44.
[8] Keller, G. (2005). Embryonic stem cell differentiation: emergence of a new era in biology and medicine. Genes Dev 19, 1129-55.
[9] Maye, P., Becker, S., Siemen, H., Thorne, J., Byrd, N., Carpentino, J. and Grabel, L. (2004). Hedgehog signaling is required for the differentiation of ES cells into neurectoderm. Dev Biol 265, 276-90.
[10] Rathjen, J., Haines, B. P., Hudson, K. M., Nesci, A., Dunn, S. and Rathjen, P. D. (2002). Directed differentiation of pluripotent cells to neural lineages: homogeneous formation and differentiation of a neurectoderm population. Development 129, 2649-61.
[11] Bain, G., Kitchens, D., Yao, M., Huettner, J. E. and Gottlieb, D. I. (1995). Embryonic stem cells express neuronal properties in vitro. Dev Biol 168, 342-57.
[12] Bibel, M., Richter, J., Schrenk, K., Tucker, K. L., Staiger, V., Korte, M., Goetz, M. and Barde, Y. A. (2004). Differentiation of mouse embryonic stem cells into a defined neuronal lineage. Nat Neurosci 7, 1003-9.
[13] Li, X. J., Du, Z. W., Zarnowska, E. D., Pankratz, M., Hansen, L. O., Pearce, R. A. and Zhang, S. C. (2005). Specification of motoneurons from human embryonic stem cells. Nat Biotechnol 23, 215-21.
[14] Gottlieb, D. I. (2002). Large-scale sources of neural stem cells. Annu Rev Neurosci 25, 381-407.
[15] Guan, K., Chang, H., Rolletschek, A. and Wobus, A. M. (2001). Embryonic stem cell-derived neurogenesis. Retinoic acid induction and lineage selection of neuronal cells. Cell Tissue Res 305, 171-6.
[16] Wichterle, H., Lieberam, I., Porter, J. A. and Jessell, T. M. (2002). Directed differentiation of embryonic stem cells into motor neurons. Cell 110, 385-97.
[17] Plachta, N., Bibel, M., Tucker, K. L. and Barde, Y. A. (2004). Developmental potential of defined neural progenitors derived from mouse embryonic stem cells. Development 131, 5449-56.
[18] Okabe, S., Forsberg-Nilsson, K., Spiro, A. C., Segal, M. and McKay, R. D. (1996). Development of neuronal precursor cells and functional postmitotic neurons from embryonic stem cells in vitro. Mech Dev 59, 89-102.
[19] Brustle, O., Jones, K. N., Learish, R. D., Karram, K., Choudhary, K., Wiestler, O. D., Duncan, I. D. and McKay, R. D. (1999). Embryonic stem cell-derived glial precursors: a source of myelinating transplants. Science 285, 754-6.
[20] Zhang, S. C., Wernig, M., Duncan, I. D., Brustle, O. and Thomson, J. A. (2001). In vitro differentiation of transplantable neural precursors from human embryonic stem cells. Nat Biotechnol 19, 1129-33.
[21] Carpenter, M. K., Inokuma, M. S., Denham, J., Mujtaba, T., Chiu, C. P. and Rao, M. S. (2001). Enrichment of neurons and neural precursors from human embryonic stem cells. Exp Neurol 172, 383-97.
[22] Lee, S. H., Lumelsky, N., Studer, L., Auerbach, J. M. and McKay, R. D. (2000). Efficient generation of midbrain and hindbrain neurons from mouse embryonic stem cells. Nat Biotechnol 18, 675-9.
[23] Colombo, E. et al. (2006). Embryonic stem-derived versus somatic neural stem cells: a comparative analysis of their developmental potential and molecular phenotype. Stem Cells 24, 825-34.
[24] Kaushansky, K. (2006). Lineage-specific hematopoietic growth factors. N Engl J Med 354, 2034-45.
[25] Kawasaki, H., Mizuseki, K., Nishikawa, S., Kaneko, S., Kuwana, Y., Nakanishi, S., Nishikawa, S. I. and Sasai, Y. (2000). Induction of midbrain dopaminergic neurons from ES cells by stromal cell-derived inducing activity. Neuron 28, 31-40.
[26] Barberi, T. et al. (2003). Neural subtype specification of fertilization and nuclear transfer embryonic stem cells and application in parkinsonian mice. Nat Biotechnol 21, 1200-7.
[27] Carpenter, M., Rao, M. S., Freed, W. and Zeng, X. (2006). Derivation and characterization of neuronal precursors and dopaminergic neurons from human embryonic stem cells in vitro. Methods Mol Biol 331, 153-67.
[28] Zeng, X. et al. (2004). Dopaminergic differentiation of human embryonic stem cells. Stem Cells 22, 925-40.
[29] Linker, C. and Stern, C. D. (2004). Neural induction requires BMP inhibition only as a late step, and involves signals other than FGF and Wnt antagonists. Development 131, 5671-81.
[30] De Robertis, E. M. and Kuroda, H. (2004). Dorsal-ventral patterning and neural induction in Xenopus embryos. Annu Rev Cell Dev Biol 20, 285-308.
[31] Piccolo, S., Sasai, Y., Lu, B. and De Robertis, E. M. (1996). Dorsoventral patterning in Xenopus: inhibition of ventral signals by direct binding of chordin to BMP-4. Cell 86, 589-98.
[32] Sasai, Y., Lu, B., Steinbeisser, H. and De Robertis, E. M. (1995). Regulation of neural induction by the Chd and Bmp-4 antagonistic patterning signals in Xenopus. Nature 376, 333-6.
[33] Lamb, T. M., Knecht, A. K., Smith, W. C., Stachel, S. E., Economides, A. N., Stahl, N., Yancopolous, G. D. and Harland, R. M. (1993). Neural induction by the secreted polypeptide noggin. Science 262, 713-8.
[34] Hemmati-Brivanlou, A., Kelly, O. G. and Melton, D. A. (1994). Follistatin, an antagonist of activin, is expressed in the Spemann organizer and displays direct neuralizing activity. Cell 77, 283-95.
[35] Hemmati-Brivanlou, A. and Melton, D. A. (1992). A truncated activin receptor inhibits mesoderm induction and formation of axial structures in Xenopus embryos. Nature 359, 609-14.
[36] Hemmati-Brivanlou, A. and Melton, D. A. (1994). Inhibition of activin receptor signaling promotes neuralization in Xenopus. Cell 77, 273-81.
[37] Smith, W. C. and Harland, R. M. (1992). Expression cloning of noggin, a new dorsalizing factor localized to the Spemann organizer in Xenopus embryos. Cell 70, 829-40.
[38] Smukler, S. R., Runciman, S. B., Xu, S. and van der Kooy, D. (2006). Embryonic stem cells assume a primitive neural stem cell fate in the absence of extrinsic influences. J Cell Biol 172, 79-90.
[39] Tropepe, V., Hitoshi, S., Sirard, C., Mak, T. W., Rossant, J. and van der Kooy, D. (2001). Direct neural fate specification from embryonic stem cells: a primitive mammalian neural stem cell stage acquired through a default mechanism. Neuron 30, 65-78.
[40] Glaser, T., Pollard, S. M., Smith, A. and Brustle, O. (2007). Tripotential differentiation of adherently expandable neural stem (NS) cells. PLoS ONE 2, e298.
[41] Conti, L. et al. (2005). Niche-independent symmetrical self-renewal of a mammalian tissue stem cell. PLoS Biol 3, e283.
[42] Benzing, C., Segschneider, M., Leinhaas, A., Itskovitz-Eldor, J. and Brustle, O. (2006). Neural conversion of human embryonic stem cell colonies in the presence of fibroblast growth factor-2. Neuroreport 17, 1675-81.
[43] Chung, Y. et al. (2008). Human embryonic stem cell lines generated without embryo destruction. Cell Stem Cell 2, 113-7.
[44] Ilic, D., Genbacev, O. and Krtolica, A. (2007). Derivation of hESC from intact blastocysts. Curr Protoc Stem Cell Biol Chapter 1, Unit 1A 2.
[45] Sato, S. M. and Sargent, T. D. (1989). Development of neural inducing capacity in dissociated Xenopus embryos. Dev Biol 134, 263-6.
[46] Grunz, H. and Tacke, L. (1989). Neural differentiation of Xenopus laevis ectoderm takes place after disaggregation and delayed reaggregation without inducer. Cell Differ Dev 28, 211-7.
[47] Krtolica, A. et al. (2007). Disruption of apical-basal polarity of human embryonic stem cells enhances hematoendothelial differentiation. Stem Cells 25, 2215-23.
[48] Bendall, S. C. et al. (2007). IGF and FGF cooperatively establish the regulatory stem cell niche of pluripotent human cells in vitro. Nature 448, 1015-21.
[49] Wang, L. et al. (2007). Self-renewal of human embryonic stem cells requires insulin-like growth factor-1 receptor and ERBB2 receptor signaling. Blood 110, 4111-9.
Catt, J. W., and Henman, M. (2000). Toxic effects of oxygen on human embryo development. Hum Reprod 15 Suppl 2, 199-206.
Chung, Y., Klimanskaya, I., Becker, S., Li, T., Maserati, M., Lu, S. J., Zdravkovic, T., Ilic, D., Genbacev, O., Fisher, S., et al. (2008). Human embryonic stem cell lines generated without embryo destruction. Cell Stem Cell 2, 113-117.
Fischer, B., and Bavister, B. D. (1993). Oxygen tension in the oviduct and uterus of rhesus monkeys, hamsters and rabbits. J Reprod Fertil 99, 673-679.
Genbacev, O., Zhou, Y., Ludlow, J. W., and Fisher, S. J. (1997). Regulation of human placental development by oxygen tension. Science 277, 1669-1672.
Ilic, D. (2006). Culture of human embryonic stem cells and the extracellular matrix microenvironment. Regen Med 1, 95-101.
Ilic, D., Caceres, E., Lu, S., Julian, P., Foulk, R., and Krtolica, A. (2009a). Normal karyotype as a determinant of successful hESC derivation. Regen Med.
Ilic, D., Giritharan, G., Zdravkovic, T., Caceres, E., Genbacev, O., Fisher, S. J., and Krtolica, A. (2009b). Derivation of human embryonic stem cell lines from biopsied blastomeres on human feeders with a minimal exposure to xenomaterials. Stem Cells Develop.
Klimanskaya, I., Chung, Y., Becker, S., Lu, S. J., and Lanza, R. (2006). Human embryonic stem cell lines derived from single blastomeres. Nature 444, 481-485.
Leese, H. J. (1995). Metabolic control during preimplantation mammalian development. Hum Reprod Update 1, 63-72.
Ma, T., Grayson, W. L., Frohlich, M., and Vunjak-Novakovic, G. (2009). Hypoxia and stem cell-based engineering of mesenchymal tissues. Biotechnol Prog. [Epub: Feb. 5, 2009]
Noda, Y., Goto, Y., Umaoka, Y., Shiotani, M., Nakayama, T., and Mori, T. (1994). Culture of human embryos in alpha modification of Eagle's medium under low oxygen tension and low illumination. Fertil Steril 62, 1022-1027.
Polak, J., Mantalaris, S., and Harding, S. E., ed. (2008). Advances in Tissue Engineering (London, Imperial College Press).

What is claimed is:

1. A method of generating cancer stem cells (CSC) or tumor initiating cells (TIC) comprising:
(a) contacting a non-cancer stem cell or a normal derivative thereof with at least one mutagen;
(b) selecting stem cell clone that displays a transformed phenotype characteristic of stem cell malignant transformation.

2. The method of claim 1 wherein the non-cancer stem cell is a human stem cell.

3. The method of claim 1 wherein the non-cancer stem cell is a human embryonic stem cell, a human adult stem cell, a human neural stem cell, or a derivative thereof.

4. The method of claim 1 wherein the cancer stem cell is an early premalignant cell, a premalignant cell, or a malignant cell.

5. The method of claim 1 wherein the mutagen comprises ICR191, 9-aminoacridine, ICR364-OH, ICR170, nitrosoguanidine, diethylsulfate, any member of an acridine family of mutagens or a derivative thereof.

6. The method of claim 1 wherein the transformed phenotype comprises cell morphology, cell growth, cell differentiation, cell polarization, cell adhesion, cell migration, or cell invasiveness.

7. The method of claim 1 wherein stem cell malignant transformation comprises an unaltered state, early premalignant state, premalignant state, malignant state, or an advanced malignant state of a stem cell.

8. A method of generating isogenic human CSC or TIC comprising deriving human CSC from human non-cancer stem cells or their derivatives of the same genetic origin.

9. The method of claim 8 wherein the non-cancer stem cell is a human embryonic stem cell, a human adult stem cell, a human neural stem cell, a human progenitor cell, or a differentiated derivative thereof.

10. The method of claim 8 wherein the cancer stem cell is an early premalignant cell, a premalignant cell, or a malignant cell.

11. The method of claim 8 wherein the genetic origin is of a type of variation selected from the group consisting of ethnicity, genetic polymorphism, predisposition to a disease, and genetic mutation.

12. The method of claim 8 wherein the deriving comprises the steps of contacting a non-cancer stem cell with at least one mutagen; selecting stem cell clones that display a transformed phenotype characteristic of stem cell malignant transformation; and isolating cells from tumor that is generated via in vivo tumorigenesis.

13. The method of claim 12 wherein the mutagen comprises ICR191, 9-aminoacridine, ICR364-OH, ICR170, nitrosoguanidine, diethylsulfate, any member of an acridine family of mutagens or a derivative thereof.

14. The method of claim 12 wherein the transformed phenotype comprises cell morphology, cell growth, cell differentiation, cell polarization, cell adhesion, cell migration, cell invasiveness, growth-factor independence, or anchorage independence.

15. The method of claim 12 wherein stem cell malignant transformation comprises an unaltered state, early premalignant state, premalignant state, malignant state, or an advanced malignant state of a stem cell.

16. A system for profiling stem cell malignant transformation (carcinogenesis) comprising
(a) a non-cancer stem cell or its derivatives
(b) isogenic non-cancer stem cell-derived cancer stem cells or tumor initiating cells with transformed phenotypes characteristic of each stage of stem cell malignant transformation
(c) a database with information on molecular and/or cellular characteristics of stem cell malignant transformation

17. The system of claim 16 wherein the non-cancer stem cell is a human stem cell.

18. The system of claim 16 wherein the non-cancer stem cell is a human embryonic stem cell, a human adult stem cell, a human neural stem cell, or a derivative thereof.

19. The system of claim 16 wherein the cancer stem cell is an early premalignant cell, a premalignant cell, or a malignant cell.

20. The system of claim 16 wherein the cancer stem cell and the non-cancer stem cell have the same genetic origin.

21. The system of claim 16 wherein the transformed phenotype comprises cell morphology, cell growth, cell differentiation, cell polarization, cell adhesion, cell migration, or tissue invasiveness.

22. The system of claim 16 wherein the stage of stem cell malignant transformation comprises an unaltered state, early premalignant state, premalignant state, malignant state, or an advanced malignant state of a stem cell.

23. The system of claim 16 wherein the database comprises information on molecular and cellular event associated with stem cell malignant transformation.

24. The system of claim 23 wherein the molecular and cellular event comprises a change in expression of a gene, enzymatic activity, telomerase activity, genomic stability, chromosomal modification, mutation, epigenetic change, cell growth, cell differentiation, cell polarization, cell adhesion, cell migration, or cell invasiveness.

25. A method of profiling stem cell malignant transformation comprising
(a) generating stage-specific cancer stem cells from isogenic non-cancer stem cells or their derivatives
(b) identifying molecular and/or cellular characteristics associated with each stage of stem cell malignant transformation
(c) generating a database with information on molecular and/or cellular characteristics of stem cell malignant transformation

26. The method of claim 25 wherein the non-cancer stem cell is a human stem cell.

27. The method of claim 25 wherein the non-cancer stem cell is a human embryonic stem cell, a human adult stem cell, a human neural stem cell, or a derivative thereof.

28. The method of claim 25 wherein the cancer stem cell is an early premalignant cell, a premalignant cell, or a malignant cell.

29. The method of claim 25 wherein the cancer stem cell and the non-cancer stem cell have the same genetic origin.

30. The method of claim 25 wherein the transformed phenotype comprises cell morphology, cell growth, cell differentiation, cell polarization, cell adhesion, cell migration, or tissue invasiveness.

31. The method of claim 25 wherein the stage of stem cell malignant transformation comprises an unaltered state, early premalignant state, premalignant state, malignant state, or an advanced malignant state of a stem cell.

32. The method of claim 25 wherein the molecular and cellular event comprises a change in expression of a gene, enzymatic activity, telomerase activity, genomic stability, chromosomal modification, mutation, epigenetic change, cell growth, cell differentiation, cell polarization, cell adhesion, cell migration, or cell invasiveness.

33. The method of claim 25 further comprising identifying molecular or cellular changes through short hairpin RNA (shRNA) gene expression.

34. A method of detecting a cancerous or pre-cancerous cell or diagnosing cancer comprising:
(a) obtaining a cell from a subject;
(b) assessing molecular and/or cellular profile of the stem cell from the subject;
(c) comparing the profile of the stem cell from the subject with a database of multi-stage stem cell carcinogenesis of claim 25.

35. The method of claim 34 wherein the subject is a human.

36. The method of claim 35 wherein the subject is a human pre-diagnosed or diagnosed with cancer.

37. The method of claim 34 wherein the cell is a tumor cell, a progenitor cell, or a circulating tumor cell.

38. The method of claim 34 wherein the cell is a human embryonic stem cell or a human adult stem cell.

39. The method of claim 34 wherein the cell is a normal cell, an early premalignant cell, a premalignant cell, a malignant cell, or an advanced malignant cell.

40. The method of claim 34 wherein the profile of the stem cells comprises properties of a normal untransformed stem cell.

41. The method of claim 34 wherein the profile of the stem cells is a gene expression profile or epigenetic profile comprising one or more tumorigenic properties selected from the group consisting of cell morphology, cell growth, cell differentiation, cell polarization, cell adhesion, cell migration, or cell invasiveness.

42. The method of claim 34 wherein the database of multi-stage stem cell carcinogenesis comprises information on molecular and/or cellular characteristics associated with each stage of stem cell malignant transformation.

43. The method of claim 42 wherein the stage of stem cell malignant transformation comprises an unaltered state, early premalignant state, premalignant state, malignant state, or an advanced malignant state of a stem cell.

44. A method for screening agent that preferentially affects a cancer stem cell relative to an isogenic non-cancer stem cell or its derivatives comprising:
(a) administering a biologically active agent to a cancer stem cell and an isogenic non-cancer stem cell or its derivatives;
(b) selecting an agent that exhibits a differential biological effect on the cancer stem cell than on the isogenic non-cancer stem cell or its derivatives.

45. The method of claim 44, wherein the agent is a small molecule, an antibody-based agent, or an RNA.

46. The method of claim 44, wherein the cancer stem cell is at any stage of stem cell carcinogenesis.

47. The method of claim 44, wherein the agents are screened using three dimensional cell culture system.

48. The method of claim 44, wherein the biological effect is a change in a biological response comprising cell proliferation, apoptosis, cell differentiation, cell migration, cell motility, cell polarization, cell adhesion, cell cytotoxicity, and/or cell cytokine secretion.

49. The method of claim 44, wherein the screening is performed in vitro.

50. The method of claim 44, wherein the screening is performed in vivo.

51. A method of generating neural stem cells (NSC) from human embryonic stem cells (hESC), comprising:
(a) culturing hESC;
(b) disrupting cell cell contact by making single-cell suspension;
(c) seeding single cells onto human xeno-free defined extracellular matrix (ECM) substrate;
(d) disaggregating the hESC colonies and disrupting cell-cell contact;
(e) growing disaggregated cells in a NSC culture medium in the presence of basic fibroblast growth factor (bFGF);
(f) dissociating adherent cells and replating the dissociated cells onto the NSC culture medium in the presence of bFGF for several passages to obtain NSC.

52. The method of claim 51, wherein the cells are disaggregated with accutase.

53. The method of claim 51, wherein the NSC culture medium is StemPro NSC SFM medium.

54. The method of claim 51, wherein the generated NSC express early neural lineage markers and pluropotency markers selected from the group consisting of nestin, Oct 3/4, Sox1, βIII tubulin, and radial glial protein 3CB₂.

55. The method of claim 51, wherein the ECM substrate is feeder cell-free.

56. The method of claim 51 further comprising seeding the obtained NSC onto poly-L-ornithine/laminin-coated surface in StemPro NSC SFM without bFGF for six weeks to induce differentiation of NSC into neural cell types.

57. The method of claim 51, wherein the obtained NSC are capable of differentiating into neurons, astrocytes, and oligodendrocytes.

58. The method of claim 51, wherein the obtained NSC are mutagenized to give rise to cancer NSC.

59. A method of generating an animal model of tumorigenesis comprising administering to an animal CSC/TIC cells expressing stable CSC/TIC phenotype generated via the method of claim 1.

60. The method of claim 59, wherein the animal model is used for screening compounds that modulate tumor growth in vivo.

61. The method of claim 59, wherein the animal is an immunocompromised host.

62. The method of claim 59, wherein the tumor arising from the CSC/TIC cells bears substantially the same molecular or cellular characteristics as the CSC/TIC cells administered to the animal.

63. The method of claim 59, wherein the CSC/TIC cells are differentiated.

64. The method of claim 59, wherein the CSC/TIC cells are transformed neural stem cells (NSC).

65. The method of claim 59, wherein the CSC/TIC cells are administered to a specific tissue or site of the animal.

66. The method of claim 65, wherein the CSC/TIC cells at a specific tissue or site give rise to tissue-specific tumor in the animal.

67. The method of claim 59, wherein the CSC/TIC cells are introduced with a reporter gene.

68. The method of claim 59 further comprising administering hESC-derived stromal cells in combination with CSC/TIC cells.

69. The method of claim 59, wherein the stable CSC/TIC phenotype comprises cell morphology, cell growth, cell differentiation, cell polarization, cell adhesion, cell migration, and/or tissue invasiveness.
